(19) United States
(12) Patent Application Publication
(10) Pub. No.: US 2020/0051661 A1
(43) Pub. Date: Feb. 13, 2020

(54) Pharmacogenomics of Intergenic Single-Nucleotide Polymorphisms and in Silico Modeling for Precision Therapy

(71) Applicant: Arizona Board of Regents on Behalf of the University of Arizona, Tucson, AZ (US)

(72) Inventors: Haiquan LI, Tucson, AZ (US); Ikbel ACHOUR, Washington, DC (US); Joanne BERGHOUT, Tucson, AZ (US); Yves A. LUSSIER, Tucson, AZ (US)

(73) Assignee: Arizona Board of Regents on Behalf of the University of Arizona, Tucson, AZ (US)

(21) Appl. No.: 16/342,913

(22) PCT Filed: Oct. 12, 2017
(86) PCT No.: PCT/US2017/056373

§ 371 Date: Apr. 17, 2019

Related U.S. Application Data
(60) Provisional application No. 62/409,727, filed on Oct. 18, 2016.

(51) Int. Cl. G16B 20/20; G16C 20/50; G16B 30/00; G16B 5/00

(52) U.S. Cl. CPC G16B 20/20; G16B 5/00; G16B 30/00; G16C 20/50

(57) ABSTRACT

Functionally altered biological mechanisms arising from disease-associated polymorphisms remain difficult to characterize when those variants are intergenic, or, fall between genes. The present invention uses computational modelling of single-nucleotide polymorphisms (SNPs) drawn from genome-wide association studies (GWAS) or other databases to identify SNP pairs, including SNP pairs where at least one SNP is outside a protein-coding region of a gene, having convergent biological mechanisms. Additional databases, including genomic databases, biological regulatory databases, and databases related to molecular function, are used to further identify and validate the similarity of the biological mechanisms of the SNP pairs. Prioritized SNP pairs having increased similarity of biological mechanisms are then used to identify disease mechanisms, candidate therapeutic drugs, and candidate therapeutic targets among downstream effectors of intergenic SNPs.

### CROSS-REFERENCE TO RELATED APPLICATIONS

This application claims the benefit of priority from U.S. Provisional Patent Application Nos. 62/409,727 filed Oct. 18, 2016, which is hereby incorporated by reference in its entirety to the extent not inconsistent herewith.

### STATEMENT REGARDING FEDERALLY SPONSORED RESEARCH OR DEVELOPMENT

This invention was made with government support under Grant No. P30 CA023074, awarded by NIH. The government has certain rights in the invention.

### BACKGROUND OF THE INVENTION

The abundance of newly discovered disease-associated polymorphisms now enables inquiries about their summative and interactive effects (Vockley et al., Mol. Genet. Metab., 2000, 71: 10-18). Since 2005, genome-wide association studies (GWAS) have reported 415,000 single-nucleotide polymorphisms (SNPs) associated with over 1,200 complex diseases and traits (Hindorff et al., Proc. Natl Acad. Sci. USA, 2009, 106: 9362-9367). From these studies, it has been learned that half of the disease-associated SNPs reside within poorly characterized intergenic regions. Although downstream effects of missense and nonsense coding SNPs can be investigated straightforwardly in cellular and animal models, effects arising from intergenic SNPs remain largely uncharacterized and are often challenging to validate experimentally using in vitro and in vivo assays.

Computational biology can potentially bridge the mechanistic gap between detecting disease-associated SNPs and providing biological interpretations of how different risk loci contribute to disease incidence and prevalence. It has been shown that systematically integrating studies of protein-protein interaction with experimentally verified disease-associated coding SNPs enables discovery of new disease-gene candidates and testable associations between biological pathways and disease (Lee et al., PLoS Comput. Biol., 2010, 6: e1000730; Li et al. JAMIA, 2012, 19: 295-305; Lim et al., Cell, 2006, 125: 801-814; Pujana et al., Nat. Genet., 2007, 39: 1338-1349; and Regan et al., JAMIA, 2012, 19: 306-316).

Other disease-mechanism-based methods have prioritized GWAS signals by leveraging prior biological knowledge inferred from the physical proximity of SNPs to gene loci or from expression quantitative loci (eQTL) associations (Holmans et al., Am. J. Hum. Genet., 2009, 85: 13-24; Subramanian et al., Proc. Natl Acad. Sci. USA, 2005, 102: 15545-15550; Wang et al., Am. J. Hum. Genet., 2007, 81: 1278-1283; Wang et al., Nat. Rev. Genet., 2010, 11: 843-854; Cookson et al., Nat. Rev. Genet., 2009, 10: 184-194; Emilsson et al., Nature, 2008, 452: 423-428; Nica et al., PLoS Genet., 2010, 6: e1000895; Nicolae et al., PLoS Genet., 2010, 6: e1000888; Westra et al., Nat. Genet., 2013, 45: 1238-1243; and Zhong et al., Mol. Syst. Biol., 2009, 5: 321).

Recent high-throughput genomics projects such as the Encyclopedia of DNA Elements (ENCODE) have extended quantitative measures of biological activity into intergenic regions (Dunham et al., Nature, 2012, 489: 57-74; and Forrest et al., Nature, 2014, 507: 462-470). These projects led to integrative genomic analyses and systemic mapping of disease-associated SNPs to regulatory elements, including enhancers, transcription factor (TF) binding sites or chromatin accessibility marks (Corradin et al., Genome Res., 2014, 24: 1-13; Farh et al., Nature, 2015, 518: 337-343; Grubert et al., Cell, 2015, 162: 1051-1065; Karczewski et al., Proc. Natl Acad. Sci. USA, 2013, 110: 9607-9612; Maurano et al., Science, 2012, 337: 1190-1195; and Schaub et al., Genome Res., 2012, 22: 1748-1759).

Nonetheless, analysis of how downstream disease mechanisms emerge from intergenic SNPs located in biologically active regulatory genomic regions remains elusive. Additionally, dramatic disparities in health outcomes disproportionately impact racial and ethnic minorities as well as women in the United States. The current understanding of the molecular mechanisms, disrupted by environmental and lifestyle stressors associated with health disparities, has not translated into clinical improvements and health equity in any substantive way. For example, this is especially evident when studying the impact of Chronic Obstructive Pulmonary Disease (COPD), which is dominated by significant ethnic and sex disparities.

GWAS have identified thousands of reproducible polymorphisms associated with diseases of complex inheritance (Hindorff et al., Proc. Natl Acad. Sci. USA, 2009, 106: 9362-9367). However, very few have led to the discovery of clinically actionable biomarkers and few, if any, have led to unveiling diseases exacerbated by the disproportionate environmental and lifestyle stressors experienced by disparate populations. It would be desirable to integrate multidimensional datasets on individual variability in genes, environment and lifestyle in efforts to better define health and disease at the individual and population levels, and importantly, to apply this knowledge to reduce health disparities.

### SUMMARY OF THE INVENTION

The present invention addresses the mechanistic gap between genetic information derived from single-nucleotide polymorphisms (SNPs) and disease pathophysiology. Aspects of the present invention include novel methods for discovering precision medicine-relevant druggable targets and pathways from non-coding DNA polymorphisms through large-scale computation and big data integration of genomic knowledge. Additionally, these pathways can be exploited to identify which patients are most likely to benefit from a targeted therapeutic intervention (precision therapy).

For many human diseases, multiple DNA variants have been associated with disease but most are located far from protein-coding gene sequences, in “intergenic” regions that have eluded functional characterization and therefore, significant clinical or research utility. However, as also disclosed herein, analyzing pairs of DNA variants together (SNP pairs), rather than independently, can reveal overlapping and convergent molecular aberrations driving disease with higher signal-to-noise and significantly increased statistical power through dimensional reduction. Although intergenic, it can be shown that many of these DNA variants have an impact on the mRNA transcript expression of near (cis) or far (trans) gene products (e.g. mRNAs and proteins) through long-range interactions of regulatory genomic regions (e.g. DNA elements, enhancers, promoters) enriched with chromatin modifications and/or regulatory factors (e.g. transcription factors, histone modification proteins). Shared mRNA targets, shared biological function of those mRNA targets (or a subset thereof), or shared regulatory genomic regions and/or factors discoverable at the site of both DNA variants can then be identified, validated, and utilized in therapeutic discovery.

Furthermore, common mechanisms discovered between SNP pairs can unveil new disease-causing mechanisms where other molecules (such as from environmental sources) may also contribute to the disease. Thus, important implications associated for this invention (SNP pair mechanisms of disease) include: (i) conferring a broad applicability to repositioning drugs for a large population and to discover new therapeutic targets or discover adverse effects; and (ii) identifying individuals carrying specific SNP(s) from analyzed SNP pair(s) within the population who are more likely to be treated effectively by targeting their common mechanism(s) (precision therapeutics) or who are more likely to suffer from adverse effects associated to mechanism(s) thereof.

In an embodiment, the present invention provides a method for identifying a drug able to therapeutically treat a disease, the method comprising: a) retrieving a list of single-nucleotide polymorphisms (SNPs) associated with one or more diseases from a first database, and generating a set of disease risk SNPs; b) retrieving a list of gene products from a second database, wherein said gene products have altered expression associated with one or more of the disease risk SNPs; c) selecting two or more disease risk SNPs which share at least one gene product having altered expression from the second database, and generating a set of one or more SNP pairs; d) analyzing the one or more SNP pairs to determine similarity of biological mechanisms between each SNP within a SNP pair, and generating prioritized SNP pairs where higher prioritized pairs have increased similarity of biological mechanisms; and e) identifying a drug able to interact with the shared gene product having altered expression from a prioritized SNP pair. The method may further comprise testing the drug for therapeutic effect against the disease.

Optionally, the first database is any genetic database (for example, a genome-wide association study (GWAS) database) and the second database is a gene/RNA expression database (for example, a Quantitative Trait Loci (eQTL) database). Preferably, the altered gene product is a protein or mRNA molecule.

In a further embodiment, analyzing the SNP pairs comprises determining whether each SNP of a SNP pair is within a protein-coding region of a gene (intragenic) or is outside a protein-coding region of a gene (intergenic); and prioritizing SNP pairs having at least one SNP outside a protein-coding region of a gene.

In a further embodiment, the step of identifying a drug comprises retrieving a list describing a plurality of drugs and molecular targets affected by each of the plurality of drugs from a drug database. Preferably, the drug is selected from a plurality of drug candidates approved to treat a different disease.

Optionally, one or more additional databases are searched for biological processes, molecular function, regulatory factors, or combinations thereof, associated with each of the disease SNPs, and the similarity of biological mechanisms are determined based, in part, on similarity of the biological processes, molecular function, regulatory factors, or combinations thereof, from the one or more additional databases. The one or more additional databases may include, but are not limited to, a gene ontology molecular function (e.g., a GO-MF) database, a gene ontology biological process (e.g., a GO-BP) database, any functional and regulatory database (such as ENCODE), or combinations thereof

In an embodiment, the present invention provides a method for simulating the effects of a therapeutic drug treatment on a patient or a group of patients, the method comprising: a) screening a tissue sample extracted from a patient selected to receive a drug for single-nucleotide polymorphisms (SNPs); b) identifying gene products associated with the SNPs from the patient tissue sample having altered expression; c) retrieving from one or more databases information on altered gene product expression associated with adverse or beneficial effects of the drug; and d) simulating the adverse or beneficial drug effect occurring when the drug is administered to the patient based on the gene products that are associated with both the SNPs from the patient tissue sample and the adverse or beneficial effects of the drug. The method may further comprise the step of changing the drug to be administered to the patient when an adverse drug effect is predicted to occur. In certain embodiments, the altered gene product is a protein or mRNA molecule. Optionally, the one or more databases comprise an expression database (e.g., a Quantitative Trait Loci (eQTL) database).

In an embodiment, the present invention provides a method for identifying a disease-associated pathway mechanism and its associated druggable targets with corresponding medication able to therapeutically treat a distinct disease sharing a similar underlying mechanism(s). In a further embodiment, the method comprises one or more of the following steps:

a) Retrieve from a first database (DATABASE 1) single-nucleotide polymorphisms (SNPs) associated with one or more diseases and generate a list of “disease risk SNPs”.

b) Retrieve from a second database (DATABASE 2) SNP to gene product relationships, and generate a list of SNPs associated with gene products (e.g. RNA molecules, transcripts, mRNA, microRNA, and proteins.)

c) Combine SNPs from lists generated in (a) and (b) to generate a list of SNPs that are associated with both disease risk and gene product annotations.

d) Combine all SNPs from (c) pairwise to generate a list of all SNP pair combinations (SNP pairs) along with all associated disease and gene product annotations.

e) Retrieve from a third database (DATABASE 3) information on linkage disequilibrium between each pair of SNPs to identify a list of SNP pairs which are inherited independently of one another at r² less than a set threshold.

f) Filter the list of SNP pairs from (d) according to their linkage disequilibrium from (e) and retain only SNP pairs which have both (i) disease and gene product annotations and (ii) are inherited independently of one another at an r² lower than a set threshold.

g) Determine the SNP pairs of independently inherited SNP pairs from (f) which share annotations of gene products according to a model of gene product overlap. This creates a list of “gene product prioritized SNP pairs” together with their shared gene products. As described herein, the “model of gene product overlap” between two SNPs is through associated gene product identity overlap, with significance calculated from the over-representation of the overlapping gene products between the SNP pairs vs those not overlapping (e.g. Fisher's Exact Test enrichment of mRNAs, permutation resampling of the gene product associations to SNPs, etc.).

h) Retrieve from a fourth database (DATABASE 4) all biological and/or all functional annotations for each gene product included in the list created by (f)

i) For those SNP pairs retained in (f), infer one or more biological and/or regulatory mechanisms for each SNP within the pair using the SNP-to-gene product expression relationships from (c) and gene product-to-function relationships from (h; DATABASE 4). This creates a list of SNP pairs where each is associated with one or more biological and/or regulatory annotations.

j) Using similarity modeling, determine the similarity between the two SNPs comprising each SNP pair according to the biological and/or regulatory annotations described in (h). This generates a list of “gene function prioritized SNP pairs” complementary to that in (g). In an embodiment, “similarity modeling” between biological and/or regulatory annotations associated to the SNP-pairs are predicted with a calculation of either: associated biological and/or regulatory function identity overlap, with significance calculated from the over-representation of biological and/or regulatory annotations between the SNP pairs vs those not overlapping (e.g. Fisher's Exact Test enrichment of mRNAs, permutation resampling of the gene product associations to SNPs, etc.), or the similarity and/or overlap of the biological and/or regulatory annotations through statistical models (e.g. information theoretic similarity measures in ontologies or directed acyclic graphs, network models in ontologies, permutation resampling, etc.).

k) For gene function prioritized SNP pairs in (j), identify the complete set of genes from DATABASE 4 that have annotations to those shared biological functions and/or regulatory mechanisms that were used to prioritize the pair. This creates a list comprising (1) gene function prioritize SNP pairs, (2) biological functions and/or regulatory mechanisms used in prioritization, and (3) a list of genes that may be putative functional effectors.

l) Retrieve from a fifth database (DATABASE 5) a list describing a plurality of drugs with drug-to-molecular target (gene product) and drug-to-disease indication relationships.

m) Combine the list of gene product prioritized SNP pairs in (g) with the DATABASE 5 (l) using associated molecular target (gene product) identity. This creates a list of drug-with-disease and SNP pair-with-diseases for each gene product prioritized SNP pair. If the disease indication(s) for a drug (l) are distinct from those annotated to one or both of the SNPs in a prioritized SNP pair, these additional diseases associated by SNP pair provide new potential disease indications for the drug.

n) Combine the list of genes described as putative functional effectors in (k) with the DATABASE 5 (l) using associated molecular target (gene product) identity. This creates a list of drug-with-disease and SNP pair-with-function-with-disease for each gene function prioritized SNP pair. If the disease indications from (l) are distinct from those annotated to one or both of the SNPs in a prioritized SNP pair, these additional diseases associated by SNP pair provide new potential disease indications for the drug.

The SNPs discussed above can include coding SNPs, SNPs in intergenic regions, or both as defined by any genomic or genetic database. In certain embodiments, the present invention further comprises testing the drug for therapeutic effect against the disease. In certain embodiments, the present invention further comprises predicting efficacy and adverse events of a treatment on an individual patient or a group of patients from two-SNP genotype.

In a further embodiment, DATABASE 1 may include but is not limited to SNP-to-disease relationships drawn from one or more databases (e.g. genome-wide association studies -GWAS, candidate association studies, or genetic databases as the Online Mendelian Inheritance in Man-OMIM proprietary databases, proprietary databases, etc.), or combinations thereof.

In a further embodiment, the SNPs associated with a gene product in DATABASE 2 are drawn from one or more additional databases. These additional databases include but are not limited to those SNPs mapped within the positional coordinates of a given gene or non-coding regions, or those associated with variation in any gene product expression levels (e.g. transcript or protein abundance) such as expression Quantitative Trait Loci (eQTL), protein abundance Quantitative Trait Loci (pQTL), or microRNA-QTL; or proprietary databases), or SNPs mapped in regulatory genomic regions interacting with coding regions of gene product such as chromatin interactions databases (e.g. derived from biological assays of 3C, 4C, 5C, ChIA-PET, etc.), or combinations thereof.

In a further embodiment, the information on linkage disequilibrium between each pair of SNPs in DATABASE 3 is drawn or calculated from one or more additional databases (e.g. HapMap, 1000 Genomes, proprietary databases, etc.), or combinations thereof.

In a further embodiment, the biological functions and/or regulatory mechanisms in DATABASE 4 are drawn from one or more additional databases (e.g. Gene Ontology molecular function and biological process annotations, Kyoto Encyclopedia of Genes and Genomes, proprietary databases, etc.) and regulatory and functional genomic annotations (e.g. ENCODE, Regulome DB, proprietary databases, etc.), or calculated from gene product expression (e.g. co-expression networks, proprietary databases, etc.), or combinations thereof.

In a further embodiment, the pharmacogenomics properties of a plurality of drugs with data on their associated molecular target (gene product) and disease indications in DATABASE 5 are drawn from one or more additional databases (e.g. PharmGKB, WikiData and DrugBank, proprietary databases, etc.), or combinations thereof.

In an embodiment, analyzing the SNP pairs comprises determining whether the genomic location of each SNP of a SNP pair is within the positional coordinates of gene, whether coding or noncoding (intragenic), or is outside of those regions (intergenic) based on one or more additional genomic or genetic databases (e.g. multi-genomic alignments, definition(s) of genomic boundaries (intragenic vs. intergenic).

In an embodiment, the present invention provides a method for simulating the effects of a therapeutic drug treatment on a patient or group of patients using two-SNP genotypes. SNPs include coding SNPs and SNPs in intergenic regions as defined by any genomic or genetic database. This method comprises one or more of the following steps:

a) Screen DNA extracted from a patient biological sample derived from liquid or solid biopsies to detect a patient-specific genotypes. As used herein, “screening DNA” includes whole genome sequencing, targeted sequencing, or genotyping at custom loci.

b) Retrieve from a database (DATABASE A) single-nucleotide polymorphisms (SNPs) associated with one or more diseases and generate a first list of “disease risk SNPs”.

c) Integrate the patient-specific genotype with disease-risk SNPs found in (b) (DATABASE A) to define patient-specific genotype disease risks.

d) Determine SNPs pairs derived from (b) composed with independently inherited SNPs which share annotations to the same gene products according to a model of gene product overlap to generate a list of “gene product prioritized SNP pairs”. Using similarity modeling, determine the similarity between the two SNPs of each SNP pair according to the biological and/or regulatory annotations to generate a list of “gene function prioritized SNP pairs”. Combine the “gene product prioritized SNP pairs” with the “gene function prioritized SNP pairs” to generate SNP pair mechanisms associated with one or more disease risks (pathophysiology-risk mechanisms).

e) Combine the patient-specific genotype disease risk from (c) with the pathophysiology-risk mechanisms of step (d) to generate patient-specific pathophysiology-risk mechanisms.

f) Retrieve from a database (DATABASE B) SNPs-to-gene product relationships, generating a list of SNPs associated with specific increased or decreased gene product expression (e.g. proteins or RNA molecules).

g) Retrieve from a database (DATABASE C) disease-to-gene product expression variations and generate a list of diseases associated with specific increased or decreased gene product expression (e.g. transcript, mRNA, microRNA, protein, etc.).

h) Retrieve from a database (DATABASE D) drugs-to-gene product expressions and generate a list of drugs associated with specific increased or decreased gene product expression upon administration or exposure (e.g. transcript, mRNA, microRNA, protein, etc.).

i) Retrieve from a database (DATABASE E) drug-to-known molecular target relationships, generating a list of drugs associated with validated biomolecules—including gene products—that interact with the drug directly, through physical binding or other modulation.

j) Retrieve from a database (DATABASE F) drugs-to-mechanistic modes of actions, defined as mechanisms of actions (MOA) with beneficial and/or mechanisms involved in adverse event(s) associated with indication(s), when available.

k) Retrieve from a database (DATABASE G) a list of gene-product-to-gene-product interactions.

l) Predict adverse event(s) or efficacy of drug(s) when drug-target(s) is or part of the mechanisms identified in the patient-specific pathophysiology-risk mechanisms in (e) based on models of drug target to pathophysiology-risk mechanisms. Optionally, the method further comprises changing the prescribed drug to the patient when an adverse effect is predicted to occur.

In a further embodiment, DATABASE A comprises SNP-to-disease relationships drawn from one or more databases, including but not limited to genome-wide association studies (GWAS), candidate association studies, Online Mendelian Inheritance in Man (OMIM) database, proprietary databases, or combinations thereof.

In a further embodiment, the SNPs associated with specified increased or decreased of any gene product expression levels (transcript or protein abundance) in DATABASE B are drawn or calculated from additional population/cell/tissue databases (e.g. expression Quantitative Trait Loci (eQTL), protein abundance Quantitative Trait Loci (pQTL), or microRNA-QTL, proprietary databases), or single to multiple patient-specific data of genotype-to-gene product expression relationships (genotype-transcriptome, proprietary databases), or combinations thereof.

In a further embodiment, the diseases are associated with specified increased or decreased gene product expression in DATABASE C are drawn or calculated from one or more additional databases (e.g. Gene Expression Omnibus, Transcriptional Signatures, ArrayExpress, proprietary, etc.), or combinations thereof.

In a further embodiment, administration of or exposure to a drug is associated with specified increased or decreased gene product expression in DATABASE D are drawn or calculated from one or more additional databases (e.g. Gene Expression Omnibus, high throughput cellular assays, proprietary databases, etc.), or combinations thereof.

In a further embodiment, drug-to-known molecular target relationships comprises generating a list of drugs associated with validated biomolecules—including gene products—that interact with the drug directly, through physical binding or other modulation in DATABASE E (e.g. PharmGKB, proprietary databases, etc.), or combinations thereof.

In a further embodiment, the mechanisms of action of the indication or of the adverse events of a drug are annotated in terms of gene product or biological function(s) and/or regulatory mechanism(s) in DATABASE F are drawn or calculated from one or more additional databases (e.g. PharmGKB, proprietary databases, etc.), or combinations thereof.

In a further embodiment, gene-product-to-gene-product interactions in DATABASE G are drawn or calculated from one or more additional databases (e.g. protein to protein interactions as BIND, REACTOME, STRING; transcription factors to gene target as TRANSFAC, microRNAs to mRNA targets as miRBASE, proprietary databases, etc.), or combinations thereof.

In a further embodiment, patient-specific pathophysiology-risk mechanisms are used for patient stratification as follows: the disease indication of a given drug matches (i) a disease associated directly with one or both SNPs in the gene product or gene function prioritized SNP pair patient-specific pathophysiology-risk mechanisms; and (ii) the molecular target (gene product) of said drug is known to be (DATABASE E) or interact with (DATABASE G) a molecular target associated to both SNPs in said pair via gene product overlap or biological function(s) and/or regulatory mechanism(s) relationships used for SNP-pair prioritization.

Potential indication or adverse event prediction(s) are based on shared mechanism of prioritized SNP pairs spanning across distinct diseases: the disease indication of a given drug matches (i) a disease associated directly with one of the SNPs of the gene product or gene function prioritized SNP pair (by DATABASE A); and (ii) the molecular target (gene product) of said drug is known to be (DATABASE E) or interact with (DATABASE G) a molecular target associated to both SNPs in said pair via gene product overlap or biological function(s) and/or regulatory mechanism(s) relationships used for SNP pair prioritization.

When the SNPs (or risk alleles such as identified using HapMap) impact the molecular target expression in the same direction (DATABASE B), the drug is predicted to provide benefits cross-disease (or with respect to disease-risk) to the patient. Patients at high risk for, or diagnosed with the second disease as a comorbid condition may be stratified into a priority cohort for this drug.

When the SNPs or identified risk alleles impact the molecular target expression in opposite directions (DATABASE B), the drug is predicted to increase adverse events related to the patient predisposition for the second disease, when more than one disease is annotated to the SNP pair. Patients at high risk for, or diagnosed with, the second disease as a comorbid condition should be contraindicated for this drug, or placed under additional monitoring. Specific adverse events relating to clinical markers of the second disease should be monitored in patients without clinical comorbidity at the time of therapy.

In a further embodiment, predictions of drug target mechanism modeling for precision drug repositioning, adverse event, and patient stratification can be conducted according to the following two methods: the disease indication of a given drug matches (i) a disease associated directly with both of the SNPs of the gene product or gene function prioritized SNP pair patient-specific pathophysiology-risk mechanisms; and (ii) the molecular target (gene product) of said drug (DATABASES E & F) matches the patient-specific pathophysiology-risk mechanisms predicted in but not known to interact with a molecular target of either SNP in said pair via gene product overlap or biological function(s) and/or regulatory mechanism(s) relationships used for SNP-pair prioritization.

When SNPs or identified risk alleles impact the expression of the overlapping gene product in the same direction (DATABASE B), and this gene product is modulated by the drug (DATABASE C, E and F), then the drug is predicted to provide benefits cross-disease (or disease-risk) to the patient.

When SNPs or identified risk alleles impact the expression of overlapping gene product in opposite direction (DATABASE B), and this gene product is targeted by the drug, then the drug is predicted to increase adverse events related to the patient predisposition of the second disease.

In a further embodiment, predictions of drug target mechanism modeling for precision drug repositioning, adverse event, and patient stratification can be conducted according to the two following methods: the disease indication of a given drug matches (i) neither of the diseases associated with the SNPs in the gene product or gene function prioritized SNP pair (by DATABASE GWAS), but (ii) the molecular target (gene product) of said drug is known to interact with a molecular target associated to both SNPs in said pair via gene product overlap or biological function(s) and/or regulatory mechanism(s) relationships used for SNP-pair prioritization.

When the SNPs or identified risk alleles impact the expression of overlapping gene product in the same direction (DATABASE B), and this gene product is targeted by the drug but in the opposite direction (DATABASE D), then the drug is predicted to provide benefits to one or both disease risks of the patient. When the direction of the said gene product expression in the presence of disease (DATABASE C) is concordant with that of the SNPs or risk alleles (DATABASE B), the confidence in the predictions is increased.

When the SNPs or identified risk alleles impact the expression of overlapping gene product in the same direction (DATABASE B), and this gene product is targeted by the drug in the same direction (DATABASE D), then the drug is predicted to produce an adverse event or increase disease risk to the patient. When the direction of the said gene product expression in the presence of disease (DATABASE C) is concordant with that of the SNPs or risk alleles (DATABASE B), the confidence in the predictions is increased.

One aspect of the invention provides software programs and systems for identifying drug candidates. One embodiment comprises a non-transitory computer-readable storage medium having a software program containing computer-executable instructions for performing a method for identifying drug candidates to treat a selected disease, wherein the method comprises the following steps: a) searching a first database for single-nucleotide polymorphisms (SNPs) associated with the selected disease and generate a set of disease SNPs; b) searching a second database for gene products with variation or change in expression associated with each of the disease SNPs; c) selecting two or more disease SNPs associated with at least one gene product reported with expression changes or variation from the second database and generate a set of one or more SNP pairs; d) analyzing the one or more SNP pairs and generate a similarity value for each SNP pair based on similarity of biological mechanisms between each SNP within the SNP pair; e) generating a set of prioritized SNP pairs where higher prioritized pairs have a higher similarity value, suggesting that SNP(s) within a said pair act on the same mechanism; and f) identifying a drug able to interact with the shared gene product of the prioritized SNP pair that is reported with altered or modulation in expression. In a further embodiment, analyzing the one or more SNP pairs comprises searching one or more additional databases for biological processes, molecular function, regulatory factors, or combinations thereof, associated with each of the disease SNPs, and determining the similarity of biological mechanisms based, in part, on similarity of the biological processes, molecular function, regulatory factors, or combinations thereof, from the one or more additional databases. Preferably, the computer-readable storage medium is a computer processor or server accessible by other computers through a computer network.

An embodiment of the invention comprises a pharmacogenomic system for identifying drug candidates to treat a disease comprising: a) a disease SNP module comprising information which identifies a plurality of SNPs associated with presence of the disease; b) an altered or modulated gene product module comprising information on gene products with altered or modulation in expression and plurality of SNPs; c) a pairing module connected to the disease SNP module and the altered gene product module able to generate a set of one or more SNP pairs where both SNPs in each pair are associated with a same gene product with altered or modulated expression; d) an analyzing module connected to the pairing module able to predict similarity of biological mechanisms between each SNP within a generated SNP pair and able to generate a set of prioritized SNP pairs with increased similarity of biological mechanisms; e) a drug and molecular target module comprising information on a plurality of drugs and molecular targets affected by each of the plurality of drugs; and f) an identification module able to identify one or more drugs from the plurality of drugs which affects a molecular target which is the same as the gene product having altered expression from a prioritized SNP pair.

In a further embodiment, the disease SNP module, altered gene product module, and drug and molecular target module are, independently from one another, part of a computer system, computer network, or software program executable by a computer processor. Preferably, the disease SNP module, altered gene product module, and drug and molecular target module are able to connect to one or more databases over a computer network. Optionally, the system further comprises display means for displaying the generated SNP pairs, prioritized SNP pairs, drugs and molecular targets, and identified drug candidates. Alternatively, or additionally, the system further comprises a means for inputting or selecting the disease, databases, and other parameters.

### BRIEF DESCRIPTION OF THE DRAWINGS

FIG. 1. A diagram illustrating drug repositioning based on discovering a new gene/protein target for a disease according to an embodiment of the present invention.

FIG. 2. A diagram illustrating drug repositioning based on discovering a new clinical syndrome, in which two diseases are caused by the same biological mechanism. Repositioning of drug (Rx) with known mechanism of action on disease Dx (Example A) and D₁ (Example B) according to an embodiment of the present invention.

FIG. 3. A diagram illustrating patient stratification with multiple diseases according to an embodiment of the present invention.

FIG. 4. Schematic of Lead SNP pair prioritization methods. (a) Lead SNP pairs analyzed in this example contain at least one intergenic SNP and are associated with one or more of 467 diseases in the NHGRI GWAS catalogue and with gene expression levels (6,301 mRNAs) derived from a lymphoblastoid cell line eQTL study. Although computed, pairs consisting of two intragenic SNPs were not the main focus of this study (blank in matrix). (b) Lead SNP pairs were prioritized and controlled with empirical scale-free networks to yield significant Lead SNP pairs sharing at least one of the three imputed biological mechanisms (highlighted squares). Biological knowledge bases refer to eQTL associations and gene ontology annotations of molecular functions and biological process. (c) Prioritized inter-inter and inter-intra Lead SNP pairs were further validated for genetic interaction using three independent association studies (GWAS and PheWAS), and for shared TFs and interacting regulatory elements using ENCODE-derived data sets.

FIG. 5. Surveyed Lead SNPs associated with mRNA expression and diseases found in prioritized Lead SNP pairs. Lead SNP pairs in this example were prioritized by mRNAs overlap, molecular function similarity or biological process similarity. (a) Input shown on the left, percentage of Lead SNPs in the prioritized SNP pairs and their associated mRNAs and diseases found among total surveyed Lead SNPs. Different P value and FDR cutoffs were applied to stratify SNP pair prioritization and percent-derived Lead SNPs (bars). Results at FDR<0.05 (406 intergenic lead SNPs, 472 intragenic lead SNPs, 4,493 mRNA and 312 diseases; blue highlight) were selected for subsequent analyses. (b) Venn diagram of Lead SNPs in the 5,011 prioritized pairs according to mRNA overlap, molecular function similarity, and biological process similarity. (c) The network illustrates the subset of Lead SNP pairs where both SNPs had been associated with the same disease prioritized only by the overlap of mRNA, molecular function (GO-MF) or biological processes (GO-BP) at FDR<0.05, excluding GO terms found by similarity. Under this criterion, 72 (out of 105) prioritized Lead SNP pairs associated with the same disease. Five additional ones found by similarity of GO terms are not shown for visualization clarity. 467 diseases were used as input.

FIG. 6. Enrichment of shared biological mechanisms among inter-inter and inter-intra Lead SNP pairs associated with the same disease. Three biological mechanisms in this example were imputed for each Lead SNP-pair: mRNA overlap (a), similarity of molecular functions (b), and similarity of biological processes (c). Prioritized inter-inter and inter-intra Lead SNP pairs (100,000 permutation resampling; FDR<0.05) were significantly enriched when SNPs were prioritized in same disease versus across distinct diseases regardless of the P value cutoffs for eQTL association (x axis). ORs for inter-inter and inter-intra Lead SNP pairs ranged from 2.9 to 12.8 (1.3×10⁻¹² [symbol] P [symbol] 6.5×10⁻⁴), 2.3 to 12.0 (3.3×10⁻¹¹ [symbol] P [symbol] 6.5×10⁻⁴) and 2.6 to 7.2 (8.4×10⁻¹⁷ [symbol] P [symbol] 0.01) (a-c, respectively). (d) Subset of rheumatoid arthritis (RA) prioritized disease network. Among the 34 surveyed RA-associated Lead SNPs via GWAS and 138 mRNAs via eQTL (Po10⁻⁴), eight Lead SNPs were identified that related to 15 mRNAs, 14 GO-MF, and 23 GO-BP. Three of the eight Lead SNPs shared the same mRNAs and/or similar functional mechanisms. Full network appears in FIG. 16, only prioritized SNP pairs at FDR<0.05 and with significant SNP-GO-SNP associations (FDR<0.05) are shown. r²=0.004 between rs6457620 and rs615672 (HapMap Phase 3).

FIG. 7. PheWAS illustrates genetic interactions in prioritized inter-inter Lead SNP pairs associated with rheumatoid arthritis. Non-additive genetic interaction of prioritized inter-inter Lead SNP pairs was confirmed in an independent population of 1,115 RA cases and 24,169 controls. (a) Overview of the PheWAS and genetic interaction validation process. (b) A synergistic effect was observed between unlinked (r²=0) SNP(1) rs6457617 and SNP(2) rs9268853. (c) An antagonistic effect was observed between almost unlinked (r²=0.017) SNP(1) rs6457617 and SNP(2) rs9272219. The upper parts of b, c provide insight into the effect size parameters of the logistic regression model. For example, genetic interaction is measured between two SNPs when the Ratio of OR of the interaction (RORᵢ) differs significantly from the value 1. Synergy corresponds to an increased RORᵢ, whereas antagony relates to its decrease. The combination of effect size parameters of each SNP taken alone (odds ratio; OR) with those of the interaction (RORᵢ) is required to estimate the OR associated with a specific set of minor and major alleles of both polymorphisms. The lower tables of b, c provide a systematic view of the specific OR and populations associated with each allelic combination of these interacting polymorphisms.

FIG. 8. Prioritized inter-inter and inter-intra Lead SNP pairs are enriched in genomic regions sharing common ENCODE-derived transcription factors (TFs) and regulatory elements. In this example, ENCODE data were used to assess the propensity of prioritized inter-inter and inter-intra Lead SNP pairs to localize in regulatory regions with the same (a) TF(s) via ChIP-seq, (b) two distinct interacting TFs (ChIP-seq and protein-protein interactions, PPI) and (c) long-range chromatin interaction properties (ChIA-PET). Enrichment of inter-inter and inter-intra Lead SNP pairs (odds ratios with 95% confidence, y axis) in regions sharing common regulatory properties were evaluated between (i) prioritized and non-prioritized Lead SNP pairs (Panel (I)), (ii) prioritized Lead SNP pairs in the same disease and across-diseases (Panel (II)). Greater ORs are observed in disease-specific SNP pairs (Panel (II) compared with Panel (I)); ORs range from 2.6 to 1998.9 (3.4×10⁻¹³⁶ [symbol] P [symbol] 5.3×10⁻⁸) in Panel (I) and 9.1 to 2249.9 (3.5×10⁻²² [symbol] P [symbol] 2.1×10⁻²) in Panel (II). Candidate inter-inter and inter-intra SNPs considered for the enrichments were associated with mRNAs by eQTL with P [symbol] 10⁻⁴ (mRNA overlap). Stringent prioritizations using empirical computations were performed on mRNA overlap, biological process similarity, molecular function similarity and in combination (merged methods). Enrichments of SNP pairs were performed using Fisher's exact test among all pairwise combinations of NHGRI disease-associated SNPs. Potential causal SNPs represented by the Lead SNPs in the pairs were included in this regulatory function study and were taken from RegulomeDB.

FIG. 9. Results of calculations of similarity between disease SNPs, enrichment of intragenic SNPs common between two distinct diseases (panel A), enriched common host genes between two disease SNPs (panel B), and similar host genes of SNPs (panel C).

FIG. 10. Workflow of an embodiment of the present invention resulting in identification of disease mechanisms shared among Lead SNPs using empirical prioritization based on SNP pairwise comparison. Input (panel A): 2,358 Lead SNPs associated with 467 diseases in NHGRI GWAS catalog and associated with 6301 mRNA expression levels in B Lymphoblastic cells by eQTL studies were considered, in pairwise, to find common biological mechanisms. Hypothesis (panel B): Surveyed Lead SNP pairs were then dichotomized into same disease and distinct disease Lead SNP pairs based on NHGRI GWAS catalog and into inter-inter, inter-intra and intra-intra pairs based on dbSNP genomic annotations. Mechanism analytics (panel C): These Lead SNP pairs (all three types) were prioritized by shared biological mechanisms via mRNA overlap derived from eQTL associations and similarity of biological process and/or molecular function based on gene ontology (GO) annotations of SNP-associated mRNAs. The prioritization was controlled by empirical resampling of SNP-mRNA associations and adjusted for multiple comparisons, in this case with an FDR<5% cutoff. Validation of prioritization (panel D): Prioritized SNP pairs were assessed for common regulatory properties derived from ENCODE data and were further validated in disease case studies.

FIG. 11. Violin plot data distribution for each biological entity derived from input data from FIG. 10. Input data includes 467 diseases, 2,358 Lead SNPs, 6,301 mRNAs, 1,635 molecular function annotations and 2,903 biological process annotations. On average (measured by median), each disease is associated with 5 SNPs; each SNP is associated with 2 mRNA transcripts by eQTL; and each mRNA is annotated with 3 molecular functions and 2 biological processes in the Gene Ontology knowledge base.

FIG. 12. Circos plot of SNP pairs prioritized within the same disease. Using the RCircos package (Zhang et al. BMC Bioinformatics 2013, 14:244), Lead SNP pairs associated within the same disease are shown: 38 inter-inter pairs (A), 42 inter-intra pairs (B), and 25 intra-intra pairs (C). Colored lines and alternating grey shaded blocks along the outer ring are used to represent chromosomes. SNPs are plotted according to chromosome and position with Lead SNP pairs joined by connecting lines. The inner ring shows synthetic blocks calculated across ten species using data from Larkin et al., Genome Research 2009, 19:770-777. Numbers in parentheses have been added next to connecting lines that map multiple Lead SNP pairs in close proximity at this resolution.

FIG. 13. Linkage and positional distance distribution of prioritized SNP pairs associated with the same diseases and mapped to the same chromosome. Eighty-one within-disease Lead SNP pairs were identified as having both SNPs on the same chromosome. Log scale of positional distance (dbSNP build 138) is plotted on the x-axis with linkage disequilibrium (HapMap Phase III CEU; Apr. 19, 2009) plotted on the y-axis for inter-inter (filled purple circles), inter-intra (open pink circles), and intra-intra (grey) Lead SNP pairs. The majority of pairs were more than 100,000 bp apart with very low LD (r²<0.01), indicative of independence. SNP pairs with r²>0.8 were excluded from analysis early in the process and therefore not part of these data, nor are SNP pairs where each mapped to a different chromosome (see FIG. 12).

FIG. 14. Enrichment of shared biological mechanisms among intergenic-intragenic Lead SNP pairs associated with the same disease. Three biological mechanisms were imputed for each SNP pair with LD r²<0.8: mRNA overlap (A), the similarity of molecular functions (B), and biological processes (C). These Lead SNP pairs were prioritized and controlled empirically (100,000 permutation resampling; FDR<0.05). Prioritized intergenic-intragenic Lead SNP pairs were found significantly enriched in the same disease than across distinct diseases (odds ratio in y-axis) under various p-value cuto-s for eQTL associations (SNP-mRNA) (P-value in x-axis). A one-tailed Fisher's Exact Test (FET) was used to measure the significance of the enrichment. The odds ratios range from 2.3 to 5.8 (3.6×10⁻⁵≤p≤0.04 for OR≥3.5), 1.4 to 5.2 (4×10⁻⁴≤p≤0.04 for OR≥3.5), and 1.6 to 3.7 (best p=8.1×10⁻⁷) for intergenic-intragenic SNP pairs prioritized by mRNA overlap (A), molecular function similarity (B), and biological process similarity (C), respectively. Panel D shows an example of the contingency table enrichment calculations for the cuto of 10⁻⁴ in Panel A (see “*”). These results demonstrate that enrichment of common biological mechanisms is recapitulated among intergenic and intragenic Lead SNPs associated with the same disease beyond pairs that are exclusively intergenic SNPs.

FIG. 15. Enrichment of shared biological mechanisms among Intragenic-Intragenic Lead SNP pairs associated with the same disease. Three biological mechanisms were imputed for each SNP pair with LD r²<0.8: mRNA overlap (A), the similarity of molecular functions (B), and biological processes (C). These Lead SNP pairs were prioritized and controlled empirically (100,000 permutation resampling; FDR<0.05). Prioritized intragenic-intragenic Lead SNP pairs were found significantly enriched in the same disease than across distinct diseases (odds ratio in y-axis) under various p-value cuto-s for eQTL associations (SNP-mRNA) (P-value in x-axis). A one-tailed Fisher's Exact Test (FET) was used to measure the significance of the enrichment.

FIG. 16. A network of mRNA overlap and associated GO MF overlap and GO BP overlap of Lead SNP pairs associated with Rheumatoid arthritis. Those prioritized at FDR<5% are highlighted. This network translates the mechanistic map at a single disease level to reflect relationships between different biological scales and across Lead SNPs: from prioritized Lead SNP pairs and their eQTL-associated mRNAs to their associated disease-mechanisms (GO-MF and GO-BP). The network was visualized using Cytoscape. The pairwise matrix (bottom) indicates each surveyed SNP pairs among those that were found prioritized at FDR<5% by mRNA overlap (purple square), by molecular function similarity (green square), and by biological similarity (orange square). The non-prioritized Lead SNP pairs are indicated by a grey square. The similarity between GO BP terms that share many genes and are hierarchically related is indicated by dotted lines. Computation of similarity is conducted by information theoretic distance.

FIG. 17. Enrichment of shared biological mechanisms among inter-inter and inter-intra Lead SNP pairs with stringent linkage disequilibrium cutoff (LD r²<0.01) associated with the same disease. Three biological mechanisms were imputed for each SNP pair with stringent LD control (r²<0.01): mRNA overlap (A), the similarity of molecular functions (B), and biological processes (C). SNP pairs were prioritized and controlled empirically (100,000 permutation resampling; FDR<0.05). Prioritized inter-inter and inter-intra SNP pairs were found significantly enriched in the same disease than across distinct diseases (odds ratio in y-axis) under various p-value cutoffs for the eQTL association (SNP-mRNA) dataset (P-value in x-axis). A one-tailed Fisher's Exact Test (FET) was used to measure the significance of the enrichment. The odds ratios range from 1.8 to 9.7 (1.3×10⁻⁵≤p≤0.02), 1.3 to 9.2 (1.1×10⁻⁴≤p≤0.02 for OR≥1.4), and 1.4 to 4.5 (best p=1.4×10⁻⁵) for inter-inter and inter-intra SNP pairs prioritized by mRNA overlap (A), molecular function similarity (B), and biological process similarity (C), respectively. This demonstrated that enrichment of common biological mechanisms among Lead SNPs associated with the same disease (FIG. 6) was an intrinsic property of the SNPs rather than the result of the linkage disequilibrium chosen.

FIG. 18. Enrichment of shared biological mechanisms among inter-inter and inter-intra SNP pairs associated with the same disease using liver-derived eQTL associations. Two biological mechanisms were imputed for each SNP pair with LD r²<0.8: mRNA overlap (A) and biological processes (B). The two mechanisms were derived from liver eQTL data, only p≤10⁻⁵ associations were available (truncated part indicated in grey regions), and various cutoffs were shown in x-axis by a log scale. SNP pairs were prioritized and controlled empirically (100,000 permutation resampling; FDR<0.05). Prioritized inter-inter and inter-intra SNP pairs were found significantly enriched in the same disease than across distinct diseases (odds ratio in y-axis) under various p-value cutoffs for the eQTL association (SNP-mRNA) dataset (P-value in x-axis). A one-tailed Fisher's Exact Test (FET) was used to measure the significance of the enrichment. The odds ratios range from 1.4 to 3.8 (6.1×10⁻³≤p≤0.05 for OR≥1.9) and 2.4 to 5.9 (1.5×10⁻¹²≤p≤0.04 for OR≥2.7) for inter-inter and inter-intra SNP pairs prioritized by mRNA overlap (A) and biological process similarity (B), respectively. These results demonstrated that the enrichment of common biological mechanisms among SNPs associated to the same disease (FIG. 6) could be extended to other tissues or cell lines derived eQTL association dataset beyond LCL.

FIG. 19: Q-Q plots indicate that inter-inter and inter-intra SNP pairs associated with the same disease showed significantly different distributions of their shared mechanisms compared to those associated across distinct diseases. In all four panels, the left skewed Q-Q plots indicate that, in each quartile, the “same disease” distribution contains more significant p-values than the “across distinct disease” distribution. (A) and (B). Quantile-Quantile plots (Q-Q plots) were generated to investigate two distributions of the significance of mRNA overlap between Lead SNP pairs: SNP pairs of the same disease vs. those of distinct diseases. (C) and (D). The Q-Q plots were generated to examine the distributions of Gene Ontology Biological Process similarity (GO-BP). In (A) and (C), the Q-Q plots were calculated according to three different p-value cutoffs of eQTL associations. In (B) and (D), the Q-Q plots were calculated according to three different SNP node degree cutoffs. Also, the Mann-Whitney U tests were calculated for each of the three curves in each panel (one-sided; shown alongside and color-coded according to each Q-Q plot curve). The significance of overlapping mRNAs and mRNA biological similarity of a Lead SNP pair was calculated empirically from 100,000 conservative permutations of the LCL eQTL associations and was adjusted by false discovery rate (overlap FDR or ITS FDR, shown in log scale in axis). Of note, the horizontal plateau of p-values are attributable to data being truncated at p=10⁵ (related to the number of permutations). SNP pairs with linkage disequilibrium r²≥0.01 were excluded from this study. Other cutoffs for eQTL data led to similar results (data not shown).

FIG. 20. Enrichment of shared biological mechanisms among inter-inter and inter-intra Lead SNP pairs associated within the same disease remains consistent following analysis using an alternative genome annotation. Intergenic and intragenic SNP assertions were calculated from dbSNP Build 138 and GENCODE version 19 (protein coding, miRNA, InRNA) with intergenic SNPs defined as at least 2000 bp 5′ and 500 bp 3′ of both protein-coding and noncoding gene coordinates. Three biological mechanisms were imputed for each SNP pair: mRNA overlap (A), the similarity of molecular functions (B), and similarity of biological processes (C). SNP pairs were prioritized and controlled empirically (100,000 permutation resampling; FDR<0.05). Prioritized inter-inter and inter-intra Lead SNP pairs were significantly enriched for biomodule similarity for increasing levels of eQTL association (SNP-mRNA) stringency. Enrichment was calculated “within disease” versus across “distinct diseases” using a one-tailed Fisher's Exact Test (FET). Comparing with that in FIG. 6, higher odds ratio were obtained. The odds ratios range from 3.0 to 25.4 (1.1×10⁻¹²≤p≤1.6×10⁻⁴), 2.4 to 23.7 (1.7×10⁻¹¹≤p≤3.2×10⁻⁴), and 2.8 to 13.9 (1.8×10⁻¹⁵≤p≤5.9×10⁻³) for inter-inter and inter-intra SNP pairs prioritized by mRNA overlap (a), molecular function similarity (b), and biological process similarity (c), respectively. This demonstrated that enrichment of common biological mechanisms among Lead SNPs associated with the same disease (FIG. 6) was an intrinsic property of the SNPs rather than the choice of a specific human reference genome annotation and gene definition.

FIG. 21. GWAS input covariates contributing to the interpretation of study results. The number of prioritized Lead SNP pairs within a disease is modestly correlated with the total number of SNPs associated by GWAS to a disease (A). Similarly, the proportion (percent) of prioritized Lead SNPs associated by GWAS to a disease is also slightly correlated with the total number of SNPs associated by GWAS to a disease—though this may be more complex (bimodal distribution) as the plot shows a smaller subset of anticorrelated patterns (B). Diseases overrepresented in GWAS studies are also overrepresented in the results (C). Percent of prioritized Lead SNP pairs within a disease increases imperceptibly with the number of GWAS studies for that disease (D). Correlations were assessed by a non-parametric test (Spearman; A, B, D) and difference by Mann-Whitney U test (C).

FIG. 22. Prioritized inter-inter and inter-intra Lead SNP pairs with linkage disequilibrium r²<0.01 are also enriched in genomic regions sharing common ENCODE-derived transcription factors (TFs) and regulatory elements. This figure examines the reproducibility of results in FIG. 8 using a more stringent LD cutoff of r²<0.01. ENCODE data were used to assess the propensity of prioritized inter-inter and inter-intra Lead SNP pairs to localize in regulatory regions with (A) the same TF(s) via ChIP-seq, (B) two distinct interacting TFs (ChIP-seq and protein-protein interactions, PPI), and (C) long-range chromatin interaction properties (ChIA-PET). Enrichment of inter-inter and inter-intra Lead SNP pairs (odds ratios with 95% confidence intervals, y-axis) in regions sharing common regulatory properties were evaluated between (i) prioritized and non-prioritized Lead SNP pairs (Panel I), (ii) prioritized Lead SNP pairs in the same disease and across-diseases (Panel II). Greater ORs are observed in disease-specific SNP pairs (Panel II compared to Panel I); ORs range from 1.2 to 7129.2 (2×10⁻¹⁶≤p≤0.02) in Panel I and 1.4 to 14140.2 (1.6×10⁻³¹≤p≤0.05; one exception) in Panel II. The odds ratios are comparable to those yielded by inter-inter and inter-intra SNP pairs of LD r²<0.08. Candidate inter-inter and inter-intra SNPs considered for the enrichments were associated with mRNAs by eQTL with p≤10⁻⁴ (mRNA overlap; grey bars). Stringent prioritizations using empirical computations were performed on mRNA overlap (mauve bars), biological process similarity (green bars), molecular function similarity (orange bars), and in combination (merged methods; yellow bars). Enrichments of SNP pairs were performed using Fisher's exact test among all pairwise combinations of NHGRI disease-associated SNPs. Potential causal SNPs represented by the Lead SNPs in the pairs were included in this regulatory function study and were taken from RegulomeDB.

FIG. 23. A network of 755 prioritized intergenic SNP pairs within disease class at FDR<0.05. 80 SNP pairs are within the same disease (previously published), 675 are within the same disease class but across distinct diseases (new). 3,115 SNP pairs prioritized cross-class are not shown. 19 SNPs were associated with two distinct diseases in distinct classes by GWAS and shown.

FIG. 24. A subset of prioritized network of disease class mechanisms containing 230 mRNAs shared between 428 SNP pairs and their associated GO mechanisms (48 GO-MFs, and 86 GO-BPs). Biological modularity of shared groups of mRNAs is associated with distinct SNPs themselves associated with distinct co-classified diseases. Not shown are the biomodules where 327 SNP-pairs are associated by distinct mRNAs to distinct but similar pathways. Names of classes are defined in FIG. 23.

FIG. 25. Details of implicated co-classified diseases through SNP pair similarity confirming shared genetic underpinning and biological mechanisms. Two classes, cancers (item #3 in FIGS. 23 and 24) and cardiovascular disease (item #6 in FIGS. 23 and 24), are shown. Disease-pairs are related by at least one out of 755 prioritized pair of Lead SNPs, each associated with a disease in the pair respectively. Previous studies have shown somatic mutations and transcriptomes can reclassify cancers molecularly. Here a new property is presented: common mechanisms of noncoding intergenic regions.

FIG. 26. Enrichment of shared biological mechanisms among 755 intergenic Lead SNP pairs associated with the same disease classes (LD cutoff r²<0.8), remains similar with more stringent LD cutoff (r²<0.01, not shown) and also remains the same when excluding the previously published 80 SNP pairs associated with the same diseases (results not shown). The subset of 280 prioritized SNP pairs comprising only intergenic-intergenic pairs also remains significant (see FIG. 29).

FIG. 27. Enrichment of common ENCODE-derived regulatory mechanisms in genomic regions of the prioritized intergenic Lead SNP pairs for disease classes. More stringent LD cutoff (R²<0.01) yielded similar results (not shown).

FIG. 28. Permutation based empirical statistics is more conservative than Fisher's Exact Test when assessing mRNA overlap (left panel) and semantic similarity of biological processes (right panel) of SNP pairs. Prioritized SNP pairs are indicated.

FIG. 29. Enrichment of shared mechanisms among the subset of intergenic-intergenic Lead SNP pairs associated with the same disease classes. Compared with FIG. 26 where intergenic-intragenic pairs were included with the same LD cutoff r²<0.8, the enrichment is higher for exclusively intergenic pairs.

FIG. 30. Nested Information theoretic calculations. The similarity between SNP pairs is calculated by three nested steps subsequently (I) similarity between two gene ontology terms (GO_{ITS}), (II) similarity between two genes (mRNA_{ITS}) using GO term similarities, and (III) similarity between two SNPs (SNP_{ITS}) using mRNA similarities

### DETAILED DESCRIPTION OF THE INVENTION

The present invention uses computational modelling of single-nucleotide polymorphisms (SNPs) drawn from genomic studies or other databases to identify SNP pairs, including SNP pairs where at least one SNP is outside a protein-coding region of a gene, having convergent biological mechanisms. Additional databases, including genomic databases, biological regulatory databases, and databases related to molecular function, can be used to further identify and validate the similarity of the biological mechanisms of the SNP pairs. Prioritized SNP pairs having increased similarity of biological mechanisms are then used to identify disease mechanisms, candidate therapeutic drugs, and candidate therapeutic targets among downstream effectors of intergenic SNPs.

Thus, the present invention enhances the biological and clinical interpretation of multiple genetic associations with disease pairs by identifying shared genes/gene products and pathways as plausible underpinning mechanisms. By further associating these disease pairs with excess comorbidity in presence of key disparity factors, an actionable framework is provided to validate biomarkers and possibly some of these mechanisms in a prospective cohort. These results can serve for identifying prognosis biomarkers for increasing disease prevention measures or to discover novel therapeutic targets.

Though the integration of multiple heterogeneous clinical and genomic datasets, the methods described herein provide a scalable process for cost-effective generation of multiple biomarker hypotheses. This contrasts significantly with conventional rate limiting wet laboratory methods.

In at least one aspect, the present invention presents innovation over previous studies and methods in several ways. Firstly, the present invention provides high throughput, unbiased clinical phenotyping and integration of heterogeneous large datasets. Using these clinical and genetic integrated datasets, how environmental or lifestyle stressors (ELSs) impact the disproportionate co-occurrence of genetically-anchored comorbidity can be identified. High throughput advanced statistical methods first identify appropriately matched control cohorts to test comorbidity associations with health disparities. Subsequently, methodology described herein allows for automated investigation of interacting ELSs contributing to excess comorbidities than conventional rate limiting manual approaches.

Secondly, pooling multiple genetic facts in genes and pathways increases the mechanistic understanding of the genetic underpinning of diseases. While single polymorphisms associated with diseases can be reproduced in multiple independent studies, the molecular mechanisms leading to pathophysiology remain obscure. By conducting pathway enrichment of multiple SNPs in disease pairs and their downstream transcriptomic and biological effects, the biological and clinical interpretability of the unadulterated disease-SNPs associations can be increased. The summative effect of pooling multiple genetic signals amplifies signal strength and is more clinically relevant.

Thirdly, the present invention allows for dimension reduction through computational genetics to characterize molecularly and clinically actionable comorbidities. There is an inherent curse of dimensionality in the systematic calculations of potential comorbidities in large electronic medical records (EMRs). Aspects of the present invention reduce the search space using disease pairs sharing genetic mechanisms, which requires formal approaches to represent disease terms stemming from genetic studies in the formal nomenclatures and ontologies of medical care. This approach enables molecular validation as well as potentially creates a clinically actionable roadmap for future biomarker development and the identification of novel therapeutic targets.

Additionally, the present invention allows for identification of molecular mechanisms of comorbidities associated with health disparity and dysregulated by the burden of stressors. A subset of predictions of ELSs can be validated thereby increasing the odds ratio of comorbid conditions in a prospective cohort to identify the mRNAs likely dysregulated and their underpinning genetic polymorphisms.

One aspect of the present invention provides the means to impute biological mechanisms to genetic disease risks located far away from protein coding regions (intergenic SNPs) to: (A) enable drug repositioning; (B) improve the efficacy of clinical trials through patient stratification and in silico modeling of a specific individual's response and/or adverse events; and (C) broaden the spectrum of drug discovery and development. Additionally, aspects of the present invention can apply to genetic risks of diseases including intergenic SNPs which count for more than two-thirds of disease SNPs reported by genome wide association studies (GWAS), and for which the biological understanding and clinical relevance remain elusive.

### Example 1—Drug Repositioning Using Inferred Biological Mechanisms from Intergenic and/or Intragenic SNPs

Putative drug targets can be any gene products for which altered or variation levels of mRNA expression were correlated with the presence of disease-associated single nucleotide polymorphisms (SNPs). By association, mRNA annotations are assimilated to corresponding gene product/protein. This invention can enable the discovery of additional gene products (proteins), modulated or altered by the presence of a patient's intergenic and/or intragenic polymorphisms that are overlooked by current methods and for which an approved drug for another disease might already exist.

To discover disease causal gene products (proteins) targetable by a pre-existing drug, three types of relationships can be considered: SNP-to-disease relationships drawn from genetic or genomic studies not limited to genome-wide association studies (GWAS); SNP-to-mRNA expression derived from expression studies not limited to expression Quantitative Trait Loci (eQTL) datasets from Genotype-Tissue Expression (GTEx) portal and/or eQTL based methods; and drug-to-molecular target relationships retrieved from publically or commercially available knowledge bases (e.g. PharmGKB, WikiData and DrugBank). High throughput functional assays can provide additional evidence of proteins targeted by a drug for a specific function.

Drug Repositioning at the Single Disease Level.

This approach implies the discovery of causal gene products (e.g., mRNA and proteins) affected in a given disease that can be targetable by drugs, FDA approved or experimentally known, to treat other diseases/indications.

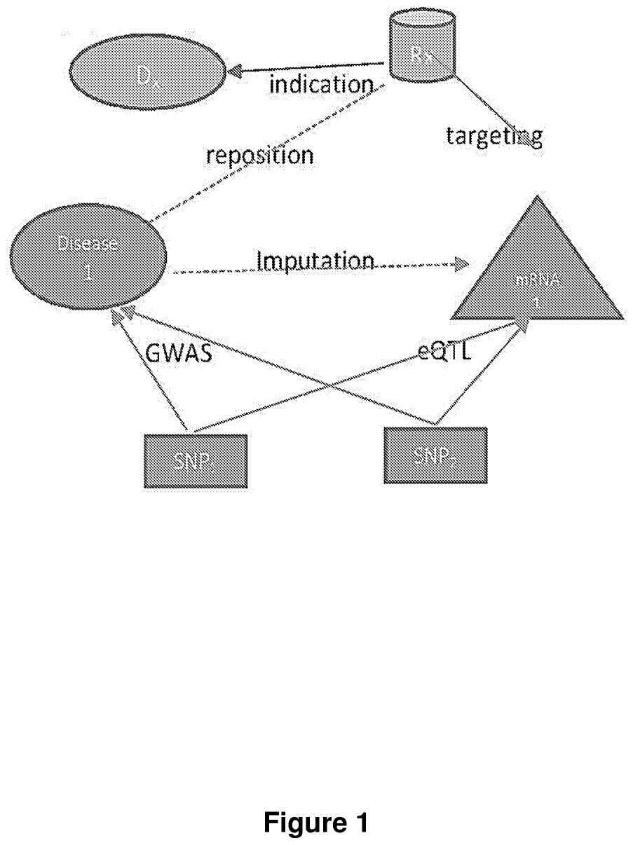

As illustrated in FIG. 1, if the gene product (e.g. mRNA 1), newly discovered by prioritized SNP pair (SNP₁ and SNP₂) as being related to disease 1 (D1), is the same as the gene product targeted by a pre-existing drug (Rx) indicated for another disease (Dx), the repositioning of Rx to treat D1 should be considered. Alternatively, the drug may target the protein coded by the mRNA 1 (related gene products).

In many cases multiple SNPs associated with the same disease (D1) may alter the same mRNA 1 and/or associated protein targets. This provides an opportunity to consider the same drug repositioned to treat different patients with the same disease but yet with different genetic risks.

While these methods address drug repositioning based on SNP pair mechanisms of disease for a population, individuals carrying the specific SNPs of a SNP pair within the population are more likely to be treated effectively by targeting this mechanism.

Drug Repositioning for a New Clinical Syndrome.

This approach implies the discovery of a gene product and/or biological mechanism affected in multiple diseases and inferred from intergenic and/or intragenic SNPs. In this instance, the method used is the eQTL similarity that identifies similar mRNA affected by distinct SNPs (e.g. eQTL) associated to distinct diseases (e.g. GWAS). This method is based on information theory and gene ontology (Tao et al., Bioinformatics, 2007, 23: i529-i538) and extends to multiple diseases the SNP-pairs similarity described for a single disease (as described further below). When an eQTL similarity is observed between two distinct SNPs, a genetic similarity can be imputed between two diseases that share a common mechanism.

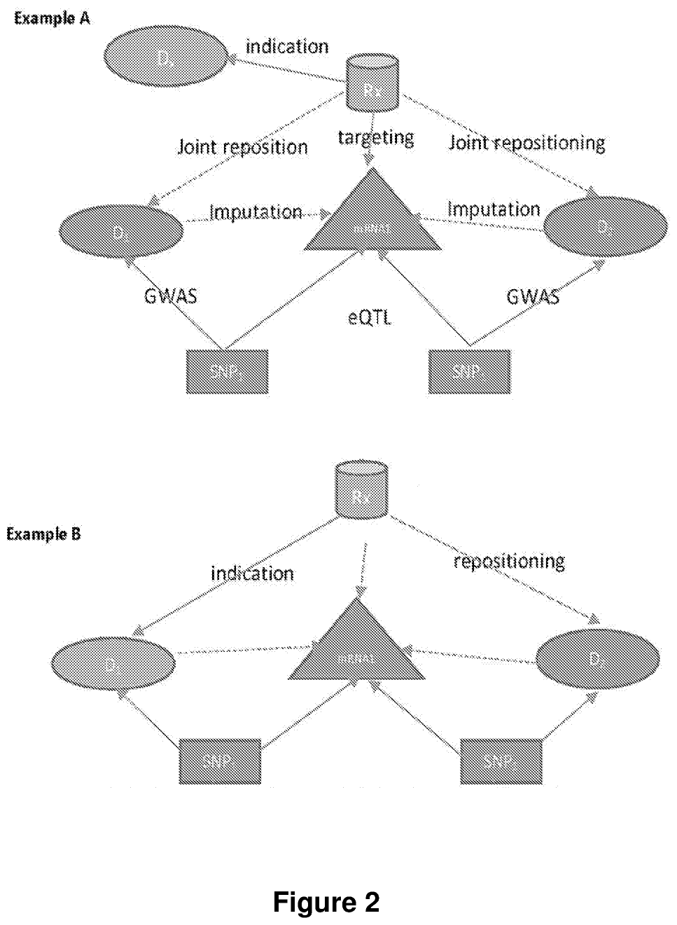

The discovery of mRNA (mRNA1) involved in a common biological mechanism downstream of a SNP₁ associated to Disease 1 (D₁) and a SNP₂ associated to Disease 2 (D₂) is illustrated in FIG. 2.

If a disease Dx treated by drug 1 (Rx) targeting the same causal protein encoded by mRNA1 and found associated to disease D₁, Rx can be considered for repositioning to treat D₁ and D₂ (FIG. 2, example A).

If a disease D₁ treated by drug 1 (Rx) targeting the same causal protein encoded by mRNA1 and found associated to disease D₂, Rx can be considered for repositioning to treat D₂ as well. (FIG. 2, example B).

While these examples address drug reposition of SNP pair mechanisms of disease for a population, individuals carrying the specific SNPs within the SNP pair within the population are more likely to be treated effectively by targeting this mechanism. Using medical records, an enrichment of comorbidity between two diseases can further confirm shared genetic mechanisms. If the indication is not identical to either disease D₁ or D₂, the drug can be repositioned for patients with both D₁ and D₂.

Efficacy of Clinical Trials Via Patient Stratification and in Silico Modeling of a Specific Individual's Response and/or Adverse Events.

This approach involves inferring the gene product/protein and/or biological mechanisms from a patient's intergenic and/or intragenic SNP pool to stratify patients who will benefit or not benefit from a given therapeutic or combination of therapeutics with or without pre-existing indications.

Most clinical trials rely on conventional designs based on testing a given drug targeting one gene product/protein on cohort of patients with the same disease without taking into account patient's genetic disease risk background. Clinical comorbidity becomes an important consideration for real patient care. However, comorbid conditions are generally excluded from clinical trials leading to under sampling the complexity of the interaction of the drug with other clinical conditions before late stage clinical trials, which insidiously increases the success of the clinical trial but increases the post-marketing risks to the populations.

If two diseases are shown to be in the same class as the result of shared underlying molecular dysfunction, particularly if predicted as a new clinical syndrome that physicians have not yet described, the present invention can provide in silico modeling of complex interactions between the drug target, the intended disease treated and potential adverse events or co-morbidity resulting from use of the drug. Additionally, this approach can lead to better clinical trial and mitigate unexpected trial failure in comorbid populations.

The present invention provides to clinical trials and drug efficacy an in silico patient stratification. Drug efficacy is likely to be substantially increased if administered to patients who carry SNPs altering a gene product/protein targeted by a drug or lead to adverse events or toxicity.

In the context of one disease, the patient's SNPs are screened using expression databases for those SNPs altering the expression of downstream mRNAs encoding for the protein targeted by the drug or encoding for other proteins indicative of toxicity. The methods described above indicate that specific up or down-regulation of RNAs are also associated to disease states. This allows imputing that a compound is associated to increasing the risk of a disease, and if this disease has an acute and severe presentation (e.g. acute bronchospasms for asthma disease), this approach also predicts an adverse event. If inadvertent, affecting a pathway that may drive chronic disease can lead to adverse events in patients, particularly if a predisposition exists (such as indicated by family history and/or genotype risk of disease). As these adverse reactions may be subtle, develop over a long periods of time, or only emerge in predisposed individuals, mechanistic insight into which body systems or metabolic pathways can highlight those potential side effects which merit closer monitoring during preclinical and clinical trials. If one of these potential side effects that are imputed develops the side-effect, then it can be determined that predisposed patients should not take the new drug. Targeted warnings and/or contraindicated conditions can be described, preventing potentially severe patient reactions and improving drug safety in clinical practice.

A drug response can be then assessed one patient at a time. Patient stratification enables the selection of patients who may benefit from a drug and rule out those that may experience side effects or toxicity; the dosage of drug can also be taken into account.

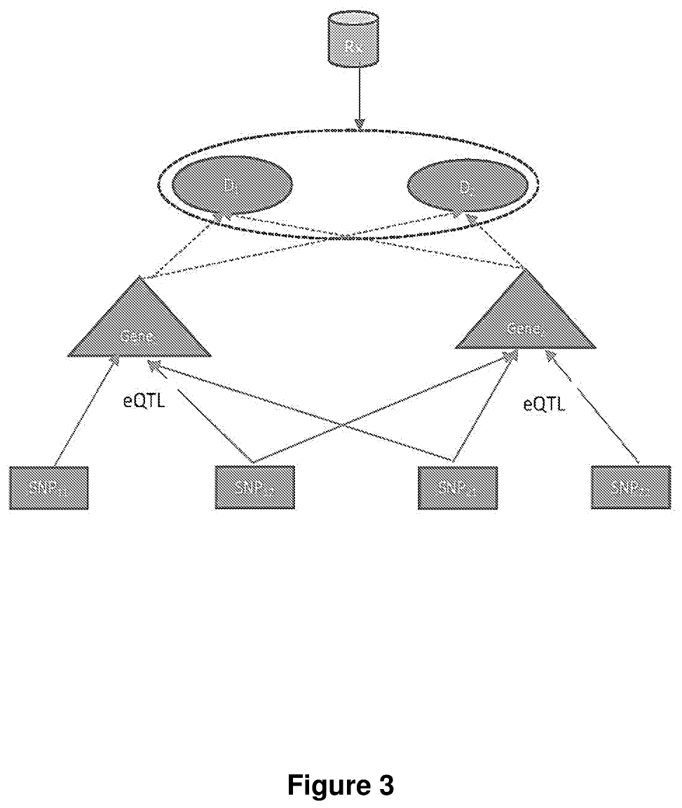

In the context of disease comorbidity (multiple diseases), new disease syndromes can be identified using SNP-SNP similarity to uncover common downstream gene products/proteins (see, for example, FIG. 3). Patients affected by two diseases with observed comorbidities are more likely to share common downstream gene products and therefore a given drug for one or the other disease can be beneficial to the patient's treatment. For each disease, the shared molecular mechanisms between pairs of SNPs can be identified. Then clusters can be identified by maximizing connections/shared mechanisms within a set of SNPs and minimizing connections/shared mechanisms across sets of SNPs with different revealed biology. The heterogeneous diseases can thus be divided into subtypes based on the clusters of biological mechanisms. Through systematic analysis of epistatic interactions and/or other biological analysis, the cooperative functions or independence between these clusters can be identified, which yields high-level clusters/subtypes. Patients are stratified accordingly by their genotype and the profiles of the clusters.

Drug discovery and development based on the discovery of new drug-targets inferred from non-coding genomic regions (e.g. intergenic SNPs). As stated above, disease risks can be associated to the presence of not only SNPs located in intragenic regions but also in intergenic regions. These so-called intergenic SNPs remain difficult to understand in terms of their impact on disease biology and their clinical relevance.

As shown in FIGS. 1-3, using integrative analysis of genetic and expression databases and/or patient's data, the prioritization of gene product (RNA or Protein) affected by intergenic SNPs can provide insight on potential protein targets for which a drug can be developed or modified. Among putative targets, additional steps can be performed to assess in silico the hubs and bottlenecks using protein-protein interaction databases (e.g. PPI).

### Example 2—Integrative Genomics Analyses Unveil Downstream Biological Effectors of Disease-Specific Polymorphisms Buried in Intergenic Regions

Functionally altered biological mechanisms arising from disease-associated polymorphisms remain difficult to characterize when those variants are intergenic, or, fall between genes. As described herein, the mechanisms by which polymorphisms contribute to disease risk can be unveiled by systematically analyzing their downstream transcriptomic effects. The functional convergence of intergenic SNPs with intragenic ones may influence the course of disease via the same mechanisms. Building on eQTL and ENCODE data, this hypothesis was approached by identifying shared molecular and biological mechanisms by which two SNPs (irrespective of their genomic location but not in linkage disequilibrium) are associated with the same disease.

A computational method was developed focusing on ascertaining and quantifying disease mechanisms of SNPs with known disease relationships from the National Human Genome Research Institute (NHGRI) GWAS catalogue (e.g., Lead SNPs), which are also associated with altered messenger RNA (mRNA) expression levels via eQTL studies. A systematic method was first devised to identify overlap and similarity of biological activities shared between every two SNPs (e.g., mRNA expression, inferred molecular function and biological processes). Second, in support of the predicted shared mechanisms between SNPs associated with the same disease, additional independent evidence were provided by: (i) exploring non-additive synergetic and antagonistic SNP-SNP interactions in GWAS of bladder cancer, Alzheimer's disease and rheumatoid arthritis (RA), and (ii) utilizing ENCODE derived data to identify Lead SNP pairs located in similar regulatory regions that might explain their shared downstream biological mechanisms. Investigations were focused on Lead SNP pairs comprised of at least one intergenic SNP.

Thus, using computational modelling of 2 million pairs of disease-associated SNPs drawn from genome-wide association studies (GWAS), integrated with expression Quantitative Trait Loci (eQTL) and Gene Ontology functional annotations, 3,870 inter-intra and inter-intra SNP pairs with convergent biological mechanisms were predicted (FDR<0.05). These prioritized SNP pairs with overlapping messenger RNA targets or similar functional annotations were more likely to be associated with the same disease than unrelated pathologies (OR412). Synergistic and antagonistic genetic interactions were additionally confirmed for a subset of prioritized SNP pairs in independent studies of Alzheimer's disease (entropy P=0.046), bladder cancer (entropy P=0.039), and rheumatoid arthritis (PheWAS case-control Po10⁻⁴). Using ENCODE data sets, it was statistically validated that the biological mechanisms shared within prioritized SNP pairs are frequently governed by matching transcription factor binding sites and long-range chromatin interactions. These results provide a ‘roadmap’ of disease mechanisms emerging from GWAS and further identify candidate therapeutic targets among downstream effectors of intergenic SNPs.

Approach Overview.

To determine the contribution of intergenic SNPs to disease risk, biological mechanisms that are common to more than one intergenic Lead SNP associated with the same disease were computationally imputed. Lead SNPs were analyzed that were associated with any of the 467 diseases in the NHGRI GWAS catalogue (Hindorff et al., Proc. Natl Acad. Sci. USA, 2009, 106: 9362-9367) and that had at least one eQTL association in the SCAN database (Gamazon et al., Bioinformatics, 2010, 26: 259-262), derived from lymphoblastoid cell lines. This yielded 2,358 Lead SNPs (1,092 intergenic) and each was paired across all possible combinations. Any pairs of SNPs that were in linkage disequilibrium with each other at r² [symbol] 0.8 using HapMap data for the CEU population were removed from the analysis (see details in Example 3).

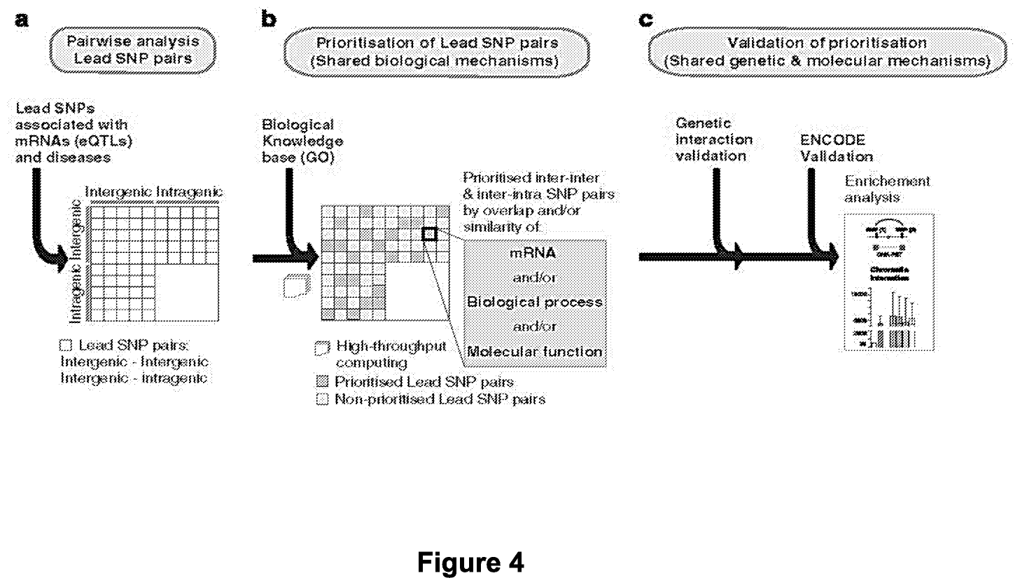

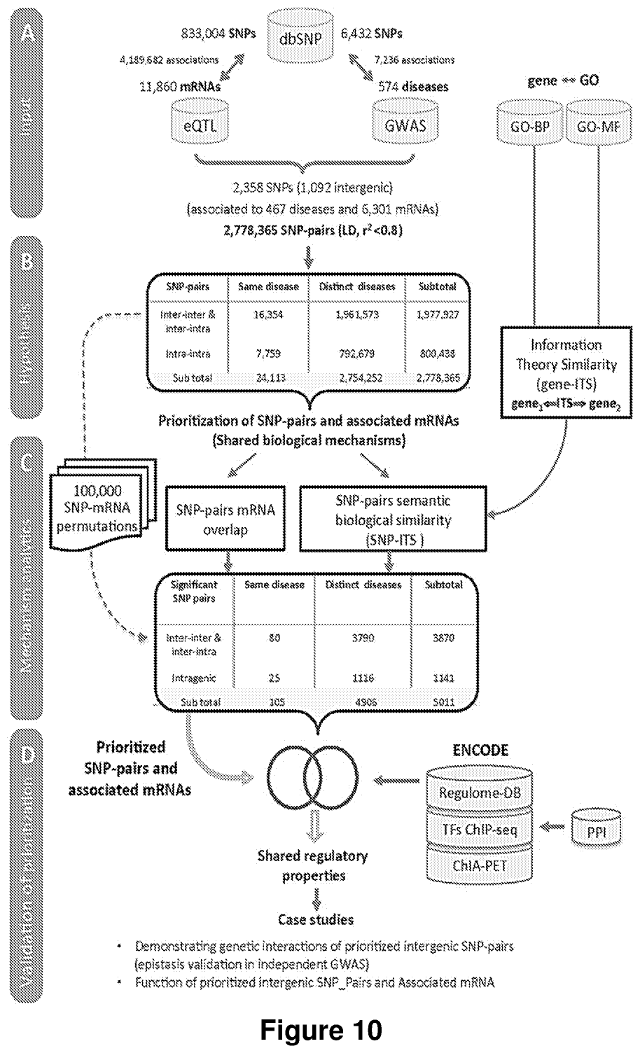

Lead SNP pairs were categorized into three groups based on assertions by dbSNP (Build 138) (Sherry et al. Nucleic Acids Res., 2011, 29: 308-311): intergenic-intergenic (inter-inter) pairs when both SNPs are at least 2,000 bp 5′ and 500 bp 3′ of protein-coding gene coordinates, intergenic-intragenic (inter-intra) pairs when one SNP is intergenic and the other is within gene coordinates, and intragenic-intragenic (intra-intra) pairs in cases where both SNPs were found within or near gene coordinates. This study focused on pairs of Lead SNPs comprised of at least one intergenic SNP (inter-inter or inter-intra), which left two million pairwise combinations (FIG. 4 and FIG. 10). For each Lead SNP, the mRNA transcripts were determined that were associated by eQTL (median 2 transcripts per SNP) and retrieved their biological processes (GO-BP) and molecular function (GO-MF) annotations from the Gene Ontology (GO 5/19/200928).

These annotations allowed prioritization of SNP pairs (inter-inter and inter-intra) on the basis of having the same or similar functional biological mechanisms, even when the exact mRNA target is distinct (e.g., receptor-ligand, signaling pathway and protein complexes). These data were then overlapped between each SNP comprising an inter-inter or inter-intra Lead SNP pair (Ashburner et al., Nat. Genet., 2000, 25: 25-29; and Tao et al., Bioinformatics, 2007, 23: i529-i538).

To evaluate the significance of imputed biological mechanisms, stringent prioritization methods were developed by mRNA overlap, GO-MF similarity and GO-BP similarity controlled empirically with scale-free networks (Lee et al., PLoS Comput. Biol., 2010, 6: e1000730; and Barabasi et al., Science, 1999, 286: 509-512) and applied these systematically to the two million surveyed Lead SNP pairs. Pairs exhibiting sufficient overlap and/or similarity at FDR<0.05 were termed ‘prioritized Lead SNP pairs’ (FIG. 4, panel b, and FIG. 10). Computationally intensive empirical calculations were required owing to random distributions being anticonservative. Enrichment analyses were then performed to assess whether shared biological mechanisms are more likely to be found among Lead SNP pairs related to the same disease rather than across distinct diseases. Leveraging ENCODE data, shared regulatory properties and molecular mechanisms at play that relate prioritized Lead SNP pairs to the same disease were evaluated. Finally, using genome-wide associations in independent data sets, it was determined that prioritized Lead SNP pairs in rheumatoid arthritis, bladder cancer and Alzheimer's disease show non-additive synergetic genetic interactions, and that long-range interactions may explain converged biological effects of inter-inter and inter-intra Lead SNPs (FIG. 4, panel c, and FIG. 10).

Substantial Associations Unveiled Between Lead SNP Pairs and Biological Mechanisms.

The three prioritization methods were first applied (statistical mRNA overlap, molecular function similarity and biological process similarity) separately to the two million surveyed Lead SNP pairs (2,358 SNPs) at False Discovery Rate (FDR)<0.05. This prioritized 5,011 total Lead SNP pairs, with 3,870 pairs containing at least one intergenic SNP (inter-inter and inter-intra pairs).

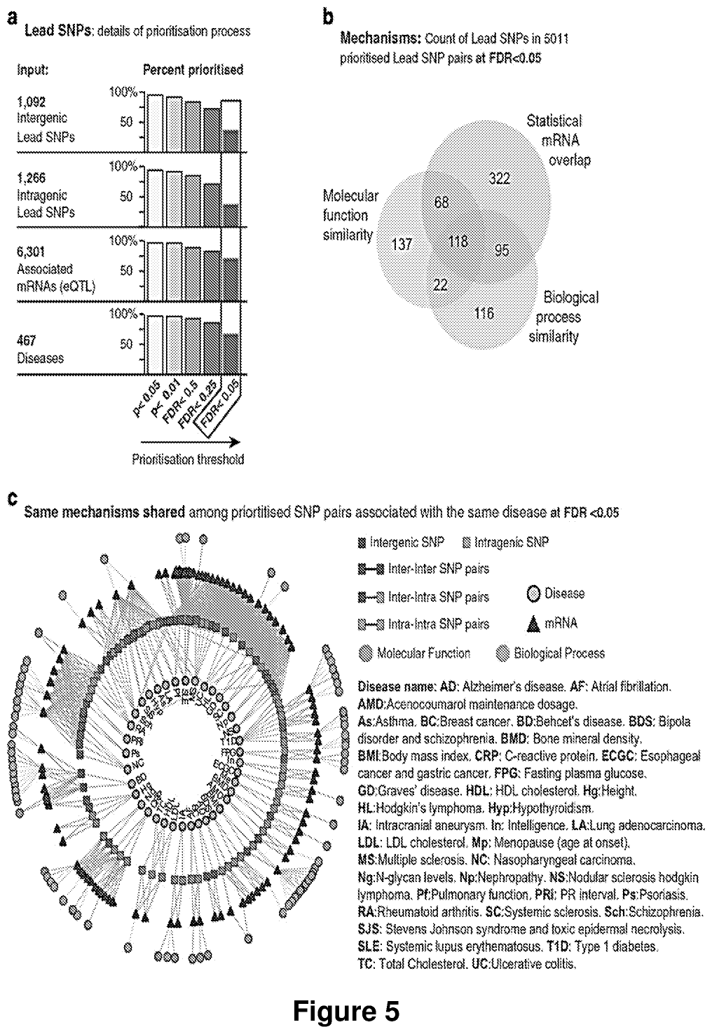

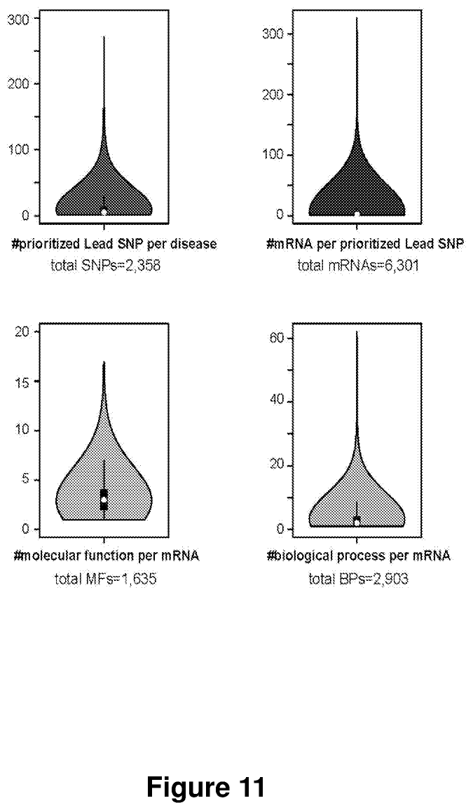

In these 5,011 SNP pairs, 406 (37% of input) intergenic Lead SNPs and 472 (37%) intragenic Lead SNPs were observed, with 4,493 (71%) associated mRNAs and corresponding to 312 (67%) diseases (FIG. 5). Details of the data distribution and composition can be found in FIG. 11. One hundred eighteen SNPs appeared in a pair that was prioritized according to all three imputed mechanisms, with 303 Lead SNPs prioritized according to at least two imputed mechanisms and the remainder of 322 (mRNA overlap), 137 (molecular function similarity) and 116 (biological process similarity) Lead SNPs were reported in pairs exhibiting a single prioritization mechanism (FIG. 5).

To visualize shared mechanisms within a given disease, prioritized SNP pairs were selected (FDR<5%) where both SNPs had been identified by association to the same disease and illustrated common mRNA targets and overlapping GO annotations (FIG. 5). These results included 43 diseases, but for visual clarity five GWAS phenotypes (Crohn's disease, immunoglobulin A levels, anorexia nervosa, prostate cancer and metabolic levels) which had highly similar but non-identical GO terms are not illustrated, although these are included in later analyses. These findings suggest that the three prioritization methods were complementary and illustrate how genetic risk of disease arises, at least in part, from systems biology properties of shared mechanisms.

Lead SNPs Sharing Biological Mechanisms are Enriched Specifically within the Same Disease.

To assess whether within-disease Lead SNPs were more likely to share biological mechanisms than SNPs associated with distinct diseases, a set of enrichment analyses was performed. Focusing on the 3,870 prioritized inter-inter and inter-intra Lead SNP pairs, 80 pairs were identified that relate to the same disease at FDR<0.05. Thirty-one SNPs were prioritized in two or more pairwise relationships for a total of 86 unique SNPs. Seven of these SNPs had exclusively cis-eQTL relationships, 44 had exclusively trans-eQTL relationships and 35 SNPs had both cis and trans-eQTLs.

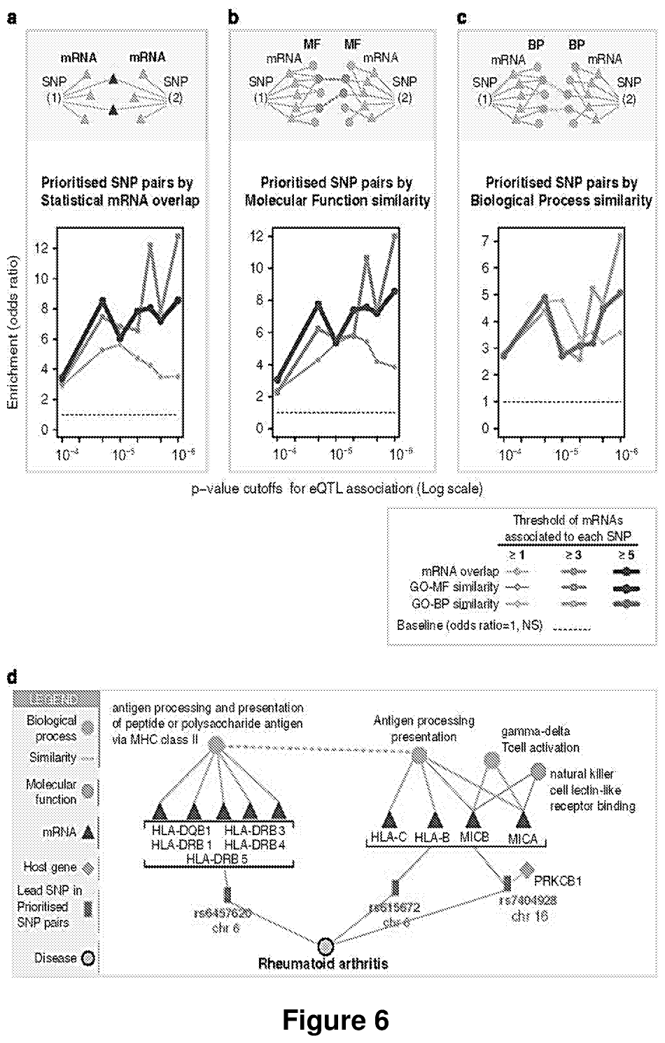

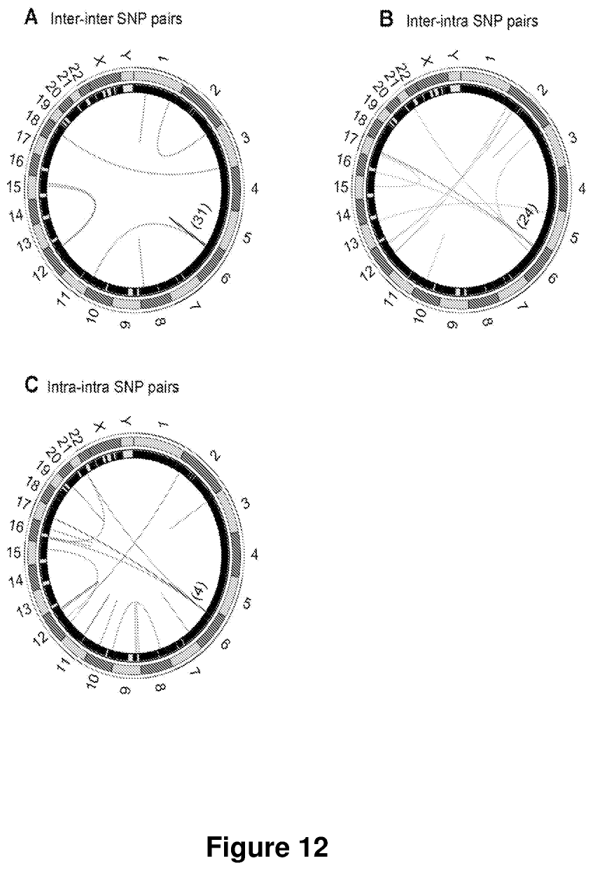

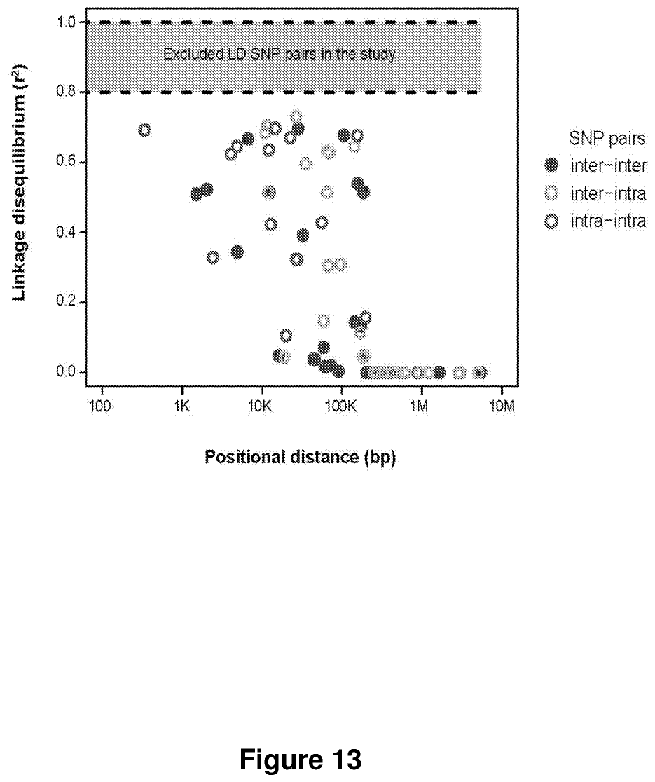

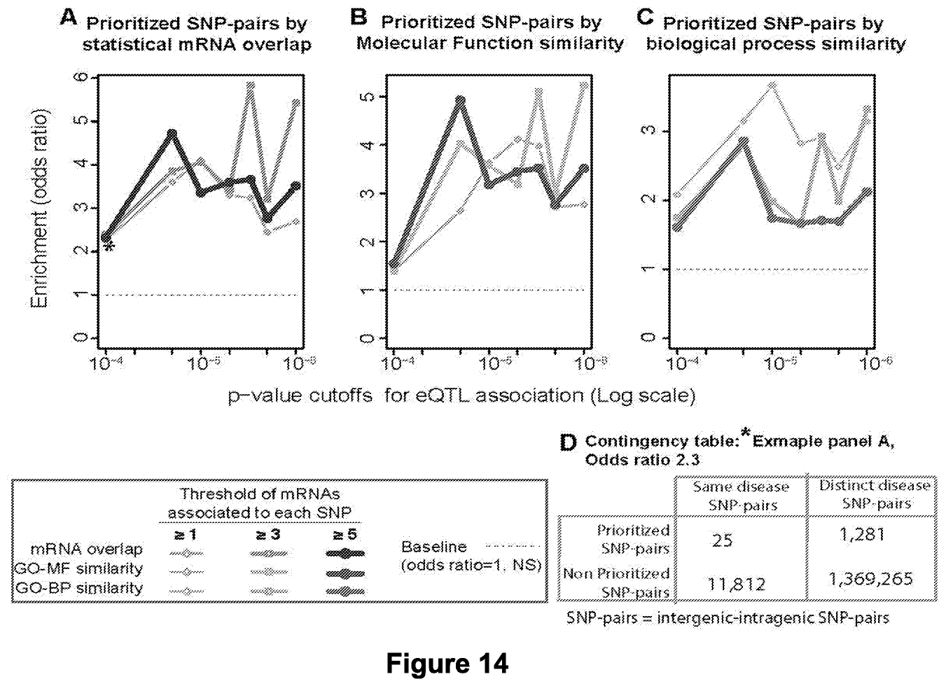

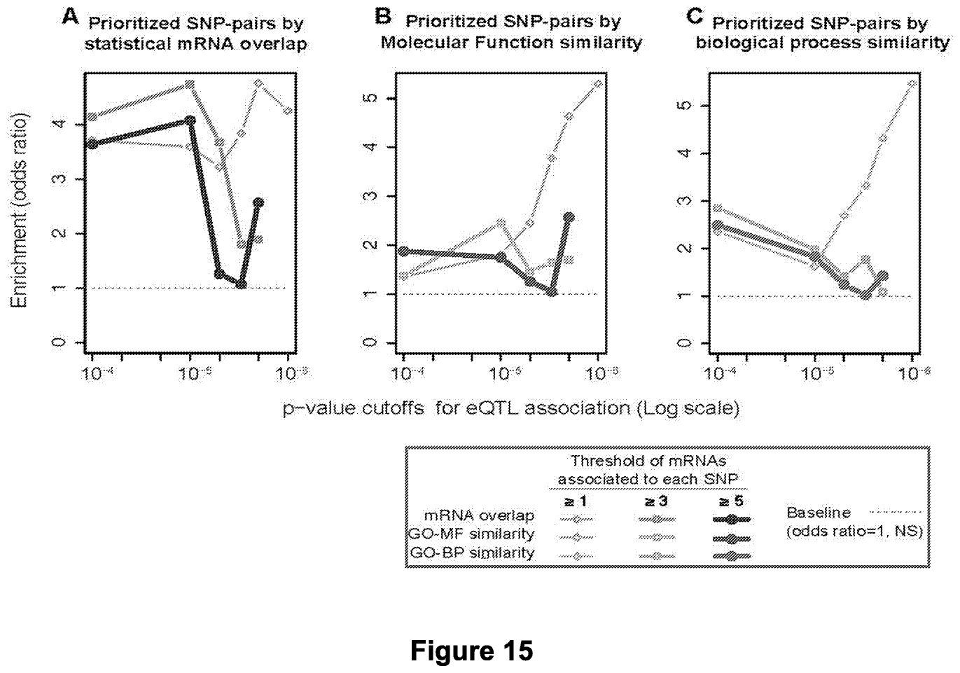

Twenty percent of the pairs (16/80) were comprised of SNPs mapping to two different chromosomes, whereas 64 pairs of SNPs were mapped to the same chromosome, although not within the same linkage disequilibrium (LD) block (FIGS. 12 and 13). Involvement of HLA in prioritized diseases was prominent, with 11% (9/80) of SNP pairs including one marker that mapped within the HLA locus (Chr6: 29-34 Mb) with the other mapping to a different chromosome, 23% (18/80) of pairs were both outside of HLA and 67% (53/80) of pairs had both SNPs within HLA. The odds ratio (OR) in favor of Lead SNPs within the same disease sharing biological mechanisms is striking when compared SNP pairs where GWAS mapping was to two distinct diseases (one-sided Fisher's Exact test; FET P=8.4×10⁻¹⁷; FIG. 6). Specifically, when using the stringent P value cutoff of eQTL association ( [symbol] 3×10⁻⁶) and multiple mRNAs associated with each Lead SNP (threshold [symbol] 3), substantial disease-specific enrichment was observed with respect to mRNA overlap (OR=12, one-sided FET P=6.1×10⁻⁹; FIG. 6, panel a), GO-MF similarity (OR=11, one-sided FET P=3.9×10⁻⁸; FIG. 6, panel b), and GO-BP similarity (OR=5.2, one-sided FET P=2.3×10⁻⁴; FIG. 6, panel c). These results were also reproduced in a subset of inter-intra Lead SNP pairs (FIG. 14), or exclusively two intragenic SNPs (FIG. 15). Even in the absence of mRNA overlap from eQTL, Lead SNP pairs with similar biological functions between their respective mRNAs remain significantly enriched with disease-specific predictions (OR=3.9, one-sided FET P=6.8×10⁻⁷).

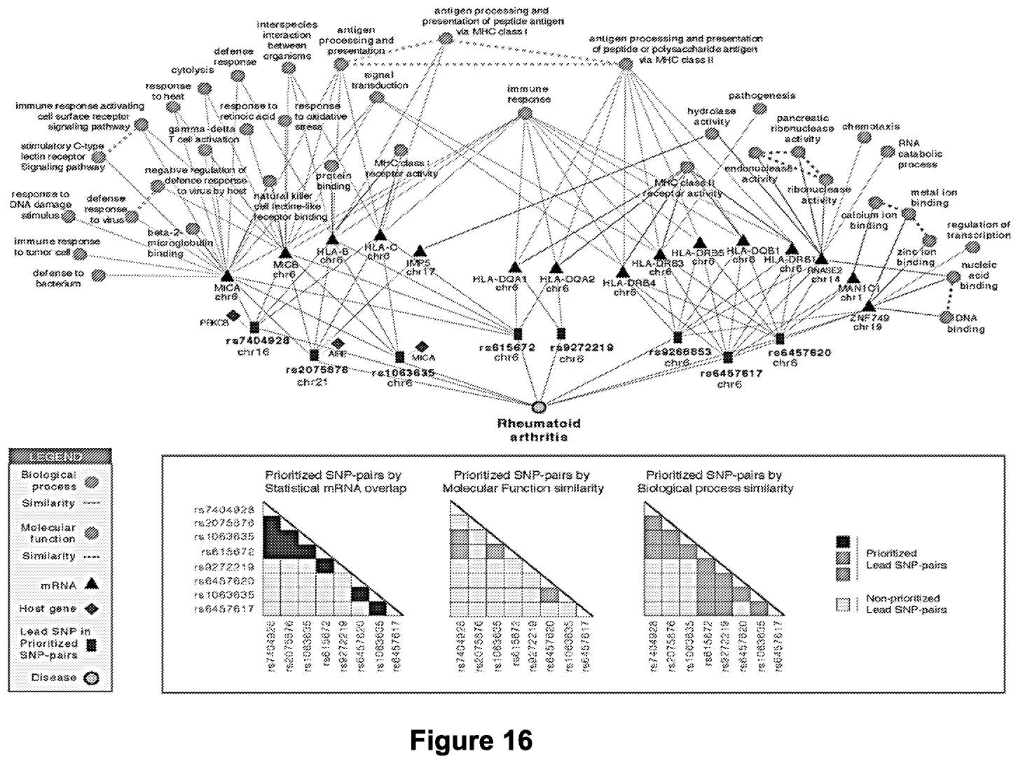

As an example of functional convergence in prioritized SNP pairs that come from the same disease, the mRNA transcript overlap, molecular function similarity and biological process similarity observed for all SNP pairs associated with RA have been illustrated (FIG. 16). Among eight Lead SNPs associated with RA, rs7404928, rs615672 and rs6457620 were prioritized by eQTL to the same mRNA transcripts (as well as non-overlapping mRNAs), and all prioritized SNPs converged towards immune response (GO:0006955) and/or antigen processing and presentation via MHC class I (GO:0002474) or class II (GO:0002586) through at least one path-including SNPs that mapped outside of the MHC region. This is consistent with what is known about the biology of RA, and the importance of antigen responses in pathology (Firestein et al., Nature, 2003, 423: 356-361).

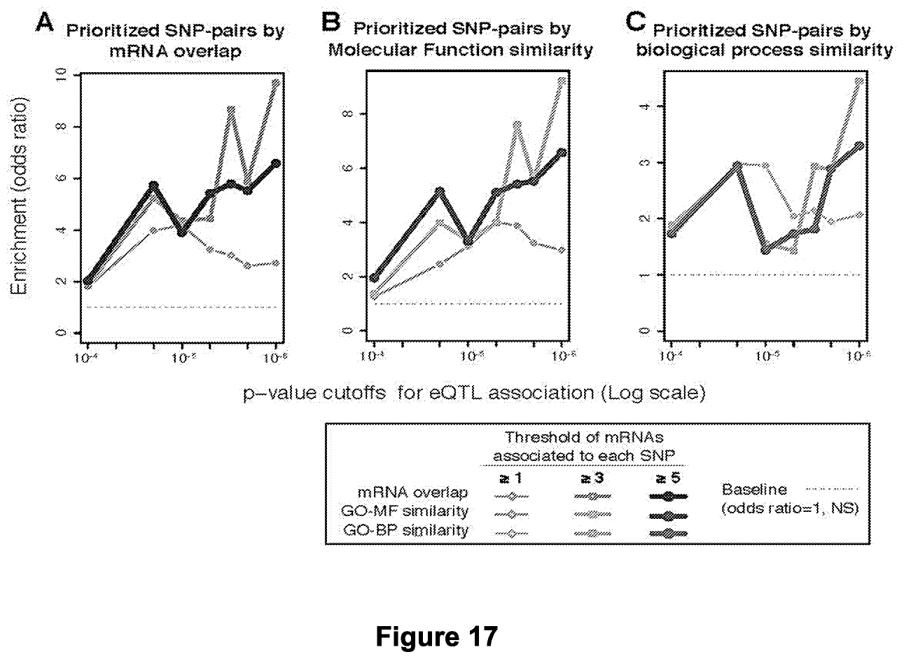

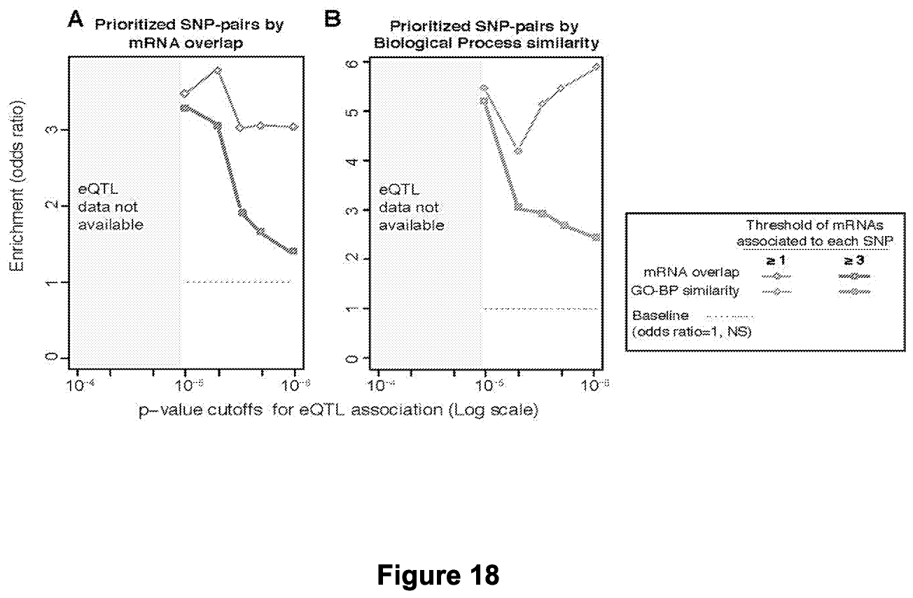

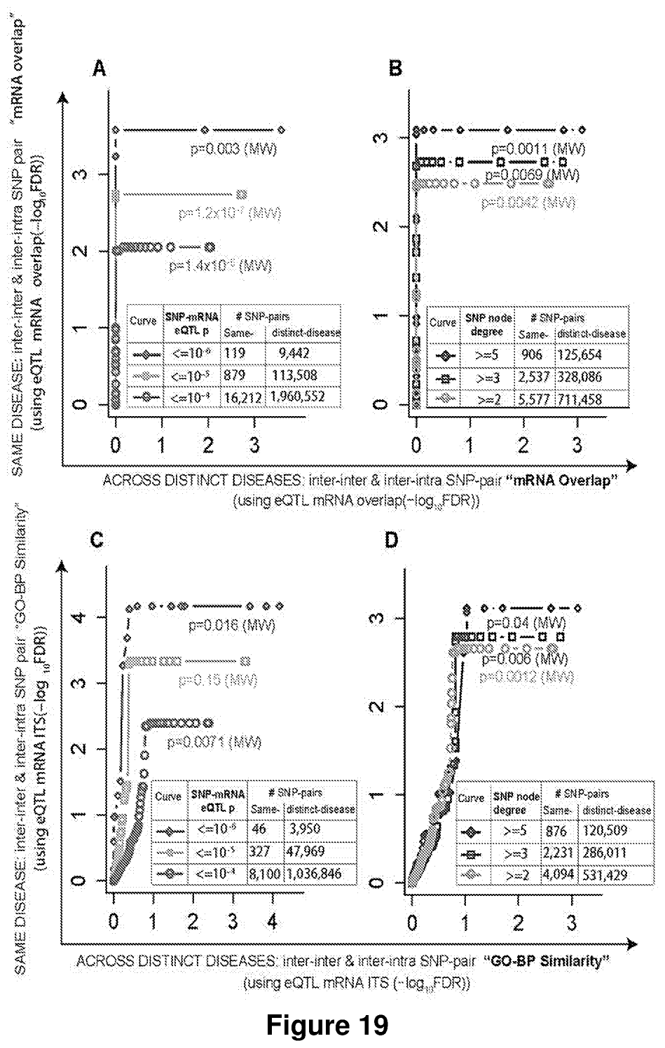

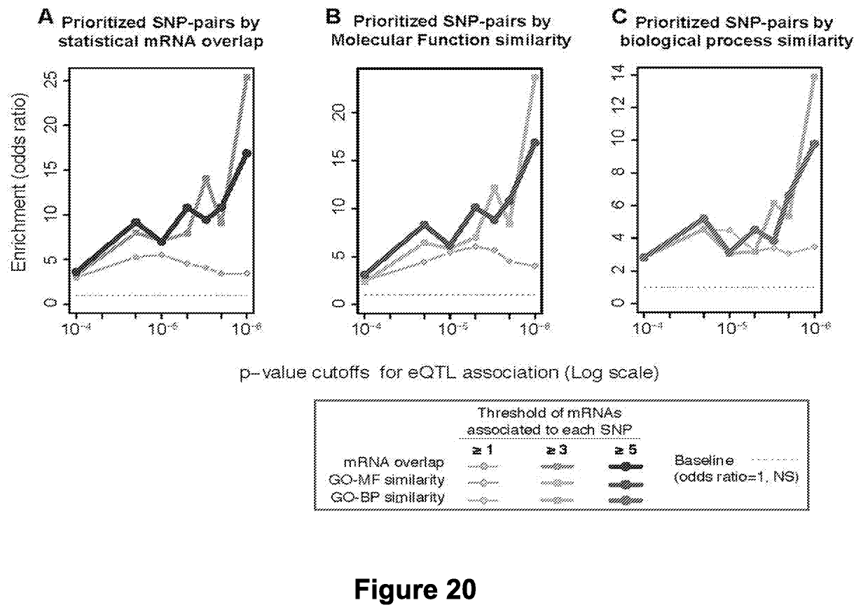

The robustness of the disease-specific enrichment found among prioritized Lead SNP pairs was further confirmed by increasing the analytical and statistical stringency. First, the LD allowance between Lead SNP pairs was decreased from r²<0.8 down to r²<0.01 (FIG. 17), which yielded very similar enrichment results. This demonstrated that the observed enrichment of shared biological mechanisms within the same disease was unlikely to be merely the result of LD. Second, the analysis was reproduced using an alternate eQTL dataset derived from liver (Fu et al., PLoS Genet., 2012, 8: el 002431), which, despite being 12-fold smaller and calculated with a more stringent P value, demonstrated that the enrichment of shared biological process mechanisms was not confounded by tissue source (FIG. 18). Interestingly, in the liver eQTL data within-disease SNP pairs were able to prioritized for hepatitis-B vaccine response and primary biliary cirrhosis, which both involve liver as a target organ. These suggest tissue-specific patterns of expression may be having important roles in addition to common patterns. Third, within-disease SNP pairs have more similarities and mRNA overlap than SNP pairs that span across distinct diseases even beyond the most rigorously prioritized results. Using all inter-inter and inter-intra Lead SNP pairs and relaxing P values by one or two orders of magnitude, data asymmetry can continue to be seen with the majority of significant P values in the same-disease results (left skew in Q-Q plots, LD r²<0.01; FIG. 19). Fourth, the enrichment analysis was performed again using an alternate reference human genome annotation, which includes coordinates for microRNA and IncRNA (GENCODE (Harrow et al., Genome Res., 2012, 22: 1760-1774) version 19; best OR=25.4, P=6.4×10⁻⁶) to establish that the results were not the result of miscategorizing SNPs within this region as intergenic (FIG. 20). Fifth, similar enrichment results were observed by applying a P<0.05 cutoff (OR=13, one-sided FET P=3.1×10⁻⁵). Overall, these controls demonstrated the approaches chosen for the pairwise comparisons and prioritizations were reproducible under multiple conditions.

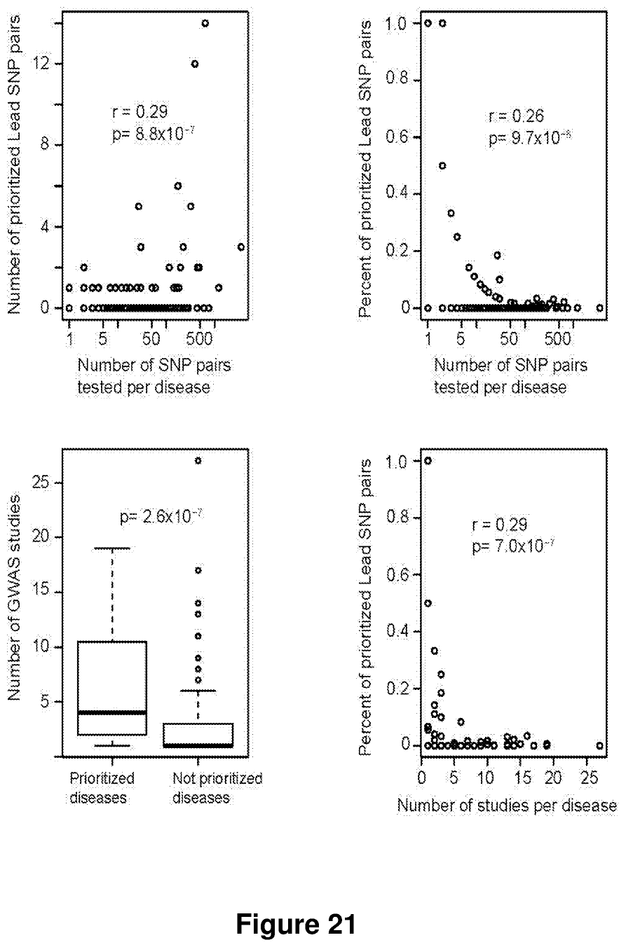

It was additionally confirmed that the enrichment results were not driven by diseases that had few GWAS SNPs. On the contrary, more SNPs and more studies per disease increased the chance of yielding more SNP pairs with shared biological mechanisms (FIG. 21).

GWAS-based evidence of non-additive synergistic genetic risk interactions among prioritized lead SNP pairs associated with bladder cancer and Alzheimer's disease. On the basis of substantial evidence for shared mechanisms among prioritized Lead SNP pairs associated with the same disease, it was hypothesized that a subset of SNPs could exhibit genetic interactions. Using independent data set of disease-SNP associations (Rothman et al., Nat. Genet., 2010, 42: 978-984; and Shen et al., Neuroimage, 2010, 53: 1051-1063), a multifactor dimensionality reduction method was applied to detect and characterize non-additive genetic interactions (Ritchie et al., Am. J. Hum. Genet., 2001, 69: 138-147; and Moore et al., Methods Mol. Biol., 2015, 1253: 301-314) among the Lead SNPs found a priori in the prioritized SNP pairs associated with bladder cancer (two pairs) and Alzheimer's disease (six pairs). The multifactor dimensionality reduction analysis revealed significant synergistic interactions for two Alzheimer's disease pairs and one of the bladder cancer pairs (Table 1). These results were significant after keeping the main effects constant and adjusting for multiple comparisons using permutation testing. In addition, SNP combinations showed evidence of synergistic effects using entropy-based measures of interaction information. This result showed that SNPs engage in cooperative or epistatic effects indicative of functionally similar mechanisms.

TABLE 1
Non-additive genetic interaction of prioritized inter-inter and
inter-intra Lead SNP pairs validated in independent GWAS studies.

| | | SNPs with | Entropy |
|---|---|---|---|
| Disease | Prioritised SNP pairs | synergistic effects | P-value |
| Alzheimer's | rs4509693-rs753129 | rs4509693- | 0.046 |
| | (chr10, inter) (chr4, | rs753129-rs7081208* | |
| | inter) rs7081208*- | | |
| | rs9331888* (chr10, | | |
| | FRMD4A) (chr8, CLU, | | |
| | MIR6843) | | |
| Bladder | rs8102137-rs1014971 | rs8102137-rs1014971 | 0.039 |
| cancer | (chr19, inter) (chr22, | | |
| | inter) | | |

Abbreviations: eQTL, expression quantitative trait loci; SNP, single-nudeotide polymorphism.
Entropy-based P values correspond to the observed statistical pattern of epistasis. SNP rs4509693 was associated with both cis and trans-eQTLs, but all other eQTL relationships were trans. Asterisks are used to indicate intragenic SNPs with host gene listed below.

Genetic Interactions of Lead SNP Pairs Prioritized by Shared Biological Mechanisms in a Phenome-Wide Association Study of RA.

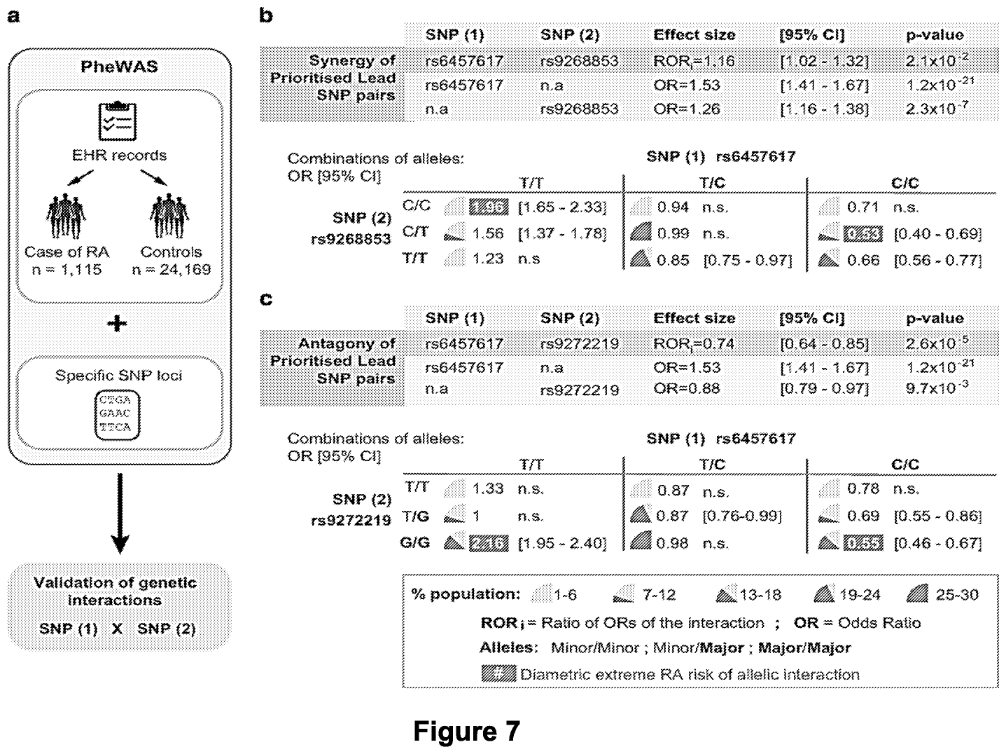

Prioritized Lead SNP pairs associated with RA were then tested using a PheWAS derived validation method for genetic interactions. SNPs were characterized in patients participating in the BioVU DNA biorepository project (Roden et al., Clin. Pharmacol. Ther., 2008, 84: 362-369) linked to an anonymous version of the Vanderbilt University Electronic Health Record (EHR), from which RA subjects were identified based on PheWAS (FIG. 7, panel a). It was first confirmed that, as expected, each Lead SNP in these pairs was actually associated with RA in this independent data set (P<0.01). Using logistic regression incorporating the ratio of ORs for genetic interaction (RORᵢ), both SNP-SNP synergy and antagonism were further identified among the RA-associated prioritized Lead SNP pairs (FIG. 7, panels b and c). For example, the Lead SNP pair comprised of rs6457617 and rs9268853 exhibited synergistic genetic interaction (RORᵢ=1.16; P=0.021; FIG. 7, panel b). For these SNPs, increased risk of RA was observed (OR=3.4, P=6.6×10⁻¹⁴) when the diametric extreme ORs of their alleles were compared (FIG. 7, panel b). In contrast, the genetic interaction of Lead SNPs rs6457617 and rs9272219 displayed an antagonistic effect (RORᵢ=0.74; P=2.6×10⁻⁵; FIG. 7, panel c). Because of the antagonism, the homozygous major alleles for rs9272219 alternatively increase or decrease the risk of RA when, respectively, combined with either the minor or major alleles for rs6457617 (OR of diametric extremes=3.2, P=2.2×10⁻¹⁶; FIG. 7, panel c). The homozygous major alleles for rs9272219 are associated with increased RA risk in the presence of the minor alleles for rs6457617(OR=2.16 versus OR=1; FIG. 7, panel c), but they are associated with the lowest risk of RA in the presence of the major alleles for rs6457617(OR=0.55, P=7.2×10⁻⁹; FIG. 7, panel c).

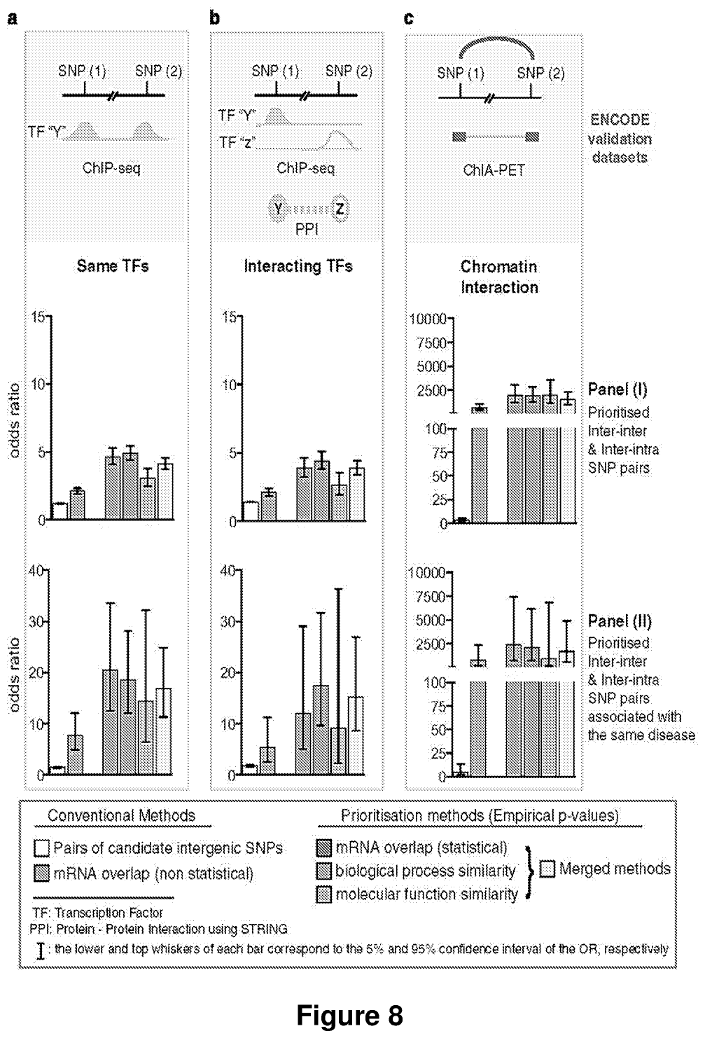

Interacting TFs and regulatory elements from ENCODE corroborate converged mechanisms prioritized between Lead SNPs. It was further hypothesized that intergenic disease-SNPs located in genomic regions surveyed for DNA-protein interactions and cis-element activities would enable the identification and characterization of the molecular regulation of prioritized biological mechanisms. ENCODE-derived biochemical assays 18 were incorporated into the study to explore three regulatory properties that Lead SNPs within each pair may share: (i) distinct SNP regions harboring the same TFs (ChIP-seq; FIG. 8, panel a), (ii) SNP regions with distinct interacting TFs (ChIP-seq and protein-protein interaction; FIG. 8, panel b) or (iii) SNP regions that physically interact via specific proteins (ChIA-PET; FIG. 8, panel c). Using RegulomeDB (Boyle et al., Genome Res., 2012, 22: 1790-1797), the study was also extended for Lead SNPs by including ENCODE-derived annotations of SNPs in strong LD (LD SNPs; r² [symbol] 0.8) with each SNP within a Lead SNP pair.

These Lead or LD SNPs may have a causative effect and/or contribute similarly to disease pathogenesis. By combining annotations, it can be seen that Lead SNP pairs with shared biological mechanisms are more likely enriched in regions with common regulatory properties than non-prioritized SNP pairs (FIG. 8). Among 3,870 inter-inter and inter-intra lead SNP pairs, 473 pairs were recovered that share genomic regions with same TFs (441 pairs), interacting TFs (223 pairs) or (31 pairs) long-range interactions. Moreover, it was demonstrated that the surveyed regulatory properties were enriched among 26 prioritized inter-inter and inter-intra SNP pairs associated with the same disease, but not across distinct diseases (FIG. 8).

Substantial enrichment of prioritized inter-inter and inter-intra Lead SNP pairs was observed in regulatory and interacting genomic regions across the three imputed biological mechanisms predicted by the methods when compared with conventional approaches, with one exception out of 12 comparisons (95% interval whiskers, FIG. 8, Panel (I)). Conventional eQTL-related methods involved identifying (i) any pair of Lead SNPs with at least one associated mRNA (P [symbol] 10⁻⁴) or (ii) straightforward (non-statistical) overlap of mRNA(s) associated with each Lead SNP of a pair. Notably, the enrichment was generally more pronounced for prioritized SNP pairs associated with the same disease, as indicated when comparing the whiskers of each prioritization method in Panel (I) to its counterpart in Panel (II) (non-overlapping whiskers, FIG. 8).

At least a threefold increase was observed in the OR for prioritized Lead SNP pairs associated with the same disease using the ENCODE ChIP-seq of transcription factors (FIG. 8, panels a and b). In addition, ChIA-PET-based analysis revealed further enrichment (OR42,500) of SNPs co-localizing with genomic regions undergoing long-range interactions mediated by chromatin-modelling DNA binding proteins of CTCF or catalyzers of DNA transcription, such as RNA polymerase II (Majumder et al., Mol. Cell Biol., 2010, 30: 4211-4223; and Ottaviani et al., Nucleic Acids Res., 2012, 40: 5262-5270). This remarkable increased enrichment is related to the nature of the ChIA-PET assays, which capture the regulatory network of transcriptional and chromatin structural activities that mirror many putative regulatory associations computed from SNPs with expressed quantitative traits (FIG. 8, panel c).

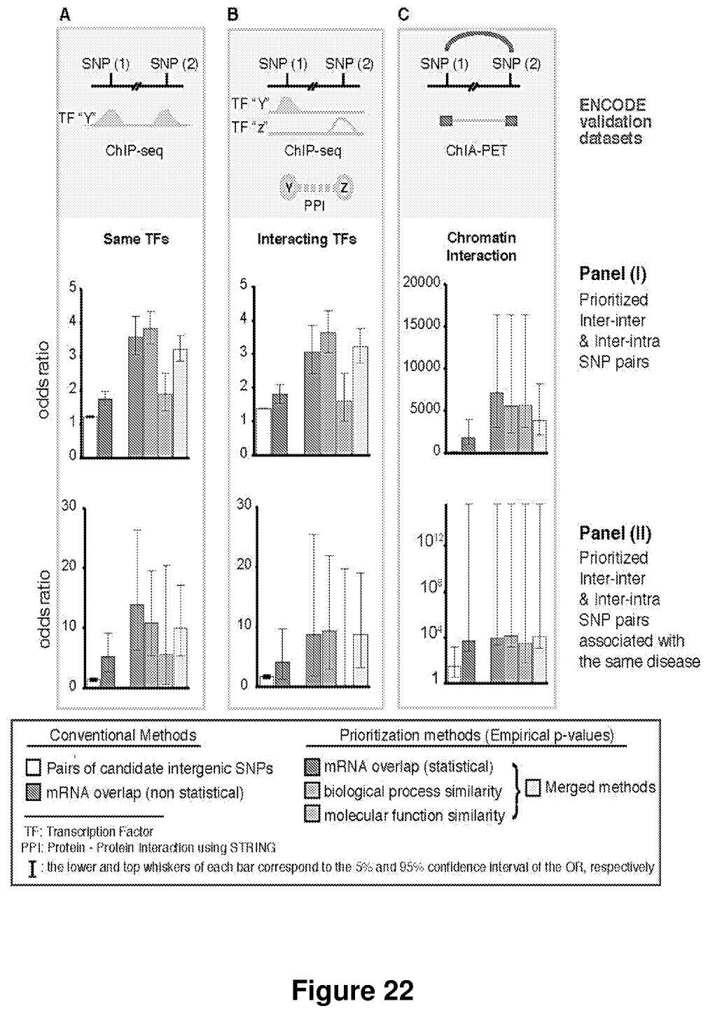

The ORs improved across every prioritization method and each of the ENCODE validation data sets when computed at an eQTL cutoff of P [symbol] 10⁻⁶ (OR>9,000, one-sided FET P=1.2×10⁻¹¹), rather than using a fixed eQTL cutoff of P [symbol] 10⁻⁴ as performed in the initial enrichment analysis illustrated in FIG. 8. In addition, ORs remain significant but slightly less when prioritizing the Lead SNP pairs at the anticonservative nominal P<0.05 (OR=896.7, one-sided FET P=3.5×10⁻¹¹). An even more stringent LD cutoff of r²<0.01 (FIG. 22) yielded comparable ORs to those from LD r²<0.8, suggesting that the convergent regulatory mechanisms between prioritized SNPs were unlikely to be the result of linkage disequilibrium. These results support the notion that SNPs related to the same disease that affect same gene expression and similar biological mechanisms are often correlated with similar functional cis- and/or trans-regulatory elements that often engage in long-range chromatin interactions such as enhancer-promoter and enhancer-enhancer interactions.

Discussion.

A computational method is described that combines different levels of genomic information (GWAS, eQTL and ENCODE) and knowledge base of gene annotations (GO) to impute biological effectors of SNPs derived from their shared biological downstream mechanisms. This study shows that intergenic and intragenic SNPs predisposing an individual to the same disease most likely affect expression of the same mRNAs, mRNAs involved in similar biological pathways or governed by similar regulatory mechanisms. Among the 2 million surveyed SNPs, and at stringent cutoff of FDR<0.05, the prioritization methods unveiled (i) 3,870 prioritized inter-inter and inter-intra Lead SNP pairs among 312 diseases that share at least one of the imputed biological mechanisms, (ii) about one third of the SNP pairs were selectively identified by at least two prioritization methods, (iii) 80 disease-specific inter-inter and inter-intra Lead SNP pairs with shared mechanisms among 32 diseases and (iv) 473 prioritized inter-inter and inter-intra SNP pairs in regions with common regulatory properties, among which 26 inter-inter and inter-intra pairs are of the same disease.

A subset of these predictions was further validated with non-additive genetic risk interactions in an independent association data set for three human diseases as well as with ENCODE-informed validations of regulatory elements. According to ENCODE regulatory data, prioritized Lead SNP pairs were also enriched for similar regulatory elements (enhancer, promoter and TFs binding sites) and were involved in the same chromatin long-range interactions. These results showed that intergenic and intragenic SNPs share disease effects through shared functionality at different level of scale of biology.

Using mRNA overlap, previous study of Fehrmann et al. recovered seven disease-specific unique SNP pairs (trans-eQTLs) at FDR<0.05 among four diseases that shared mRNAs with converged biological pathways (Fehrmann et al., PLoS Genet., 2011, 7: el 002197). This study shows that the prioritization methods were able to recover substantially more predictions by GO-BP and GO-MF similarity to identify shared mechanisms for SNP pairs without mRNA overlap. This suggests successful enrichment for those intergenic SNPs that reveal a functional impact on disease pathology.

A high number of prioritized Lead SNP pairs related to immune related loci (e.g., MHC/HLA) and their downstream activities was also observed, which is consistent with the well-described role for HLA and inflammatory processes in many complex diseases, including those studied by GWAS. It is possible that these are over-represented here due to the nature of the lymphoblastoid cell lines used for eQTL studies and their context-specific stimulations linked to particular diseases (Nica et al., PLoS Genet., 2010, 6: e1000895; and Fehrmann et al., PLoS Genet., 2011, 7: e1002197). Although many studies have reiterated such observations, neither consensus nor guidelines regarding the optimal cell lineage from which to derive eQTL associations that are most qualified for imputing disease-specific pathogenesis has been established. However, numerous eQTL and genomic annotation-based studies showed that analyzing multiple cell types could uncover novel mechanisms and biomodules that explain organs or tissue system implications in overall disease pathology (Karczewski et al., Proc. Natl Acad. Sci. USA, 2013, 110: 9607-9612; Arvey et al. Genome Res., 2012, 22: 1723-1734; Lee et al., Science, 2014, 343: 1246980; and Makinen et al., PLoS Genet., 2014, 10: e1004502).

Future directions for identifying biomodules from SNPs could optionally involve the use of unbiased gene sets such as those obtained by co-expression networks (Barabási et al., Nat. Rev. Genet., 2011, 12: 56-68; and Ramos et al., Genomics, 2007, 90: 176-185) or computational gene similarity measures (Griffiths et al., Proc. Natl Acad. Sci. USA, 2004, 101 (Suppl 1): 5228-5235). These prioritization statistics can also be applied in a targeted manner to a given disease rather than the GWAS catalogue as a whole, where a specific disease-relevant eQTL dataset may be obtained and less stringent nominal P values can be used for biomodule discovery without as much need for multiple testing correction. Further investigation in this direction is supported by independent prioritization of SNP pairs associated with liver diseases (Primary biliary cirrhosis and Hepatitis B vaccine response) when using the liver eQTL data set.

Finally, this current study was computationally intensive as the empirical resampling was conducted homogeneously across pairs. The algorithms can be optimized by conditioning the resampling according to SNP pairs and dynamically ending the resampling when P values observed are non-significant. These improvements should allow to investigate further the effect of eQTL derived from cell types more relevant to specific diseases, such as those available in Genotype-Tissue Expression data sets GTEx (Ardlie et al., Science, 2015, 348: 648-660).

Previous computational studies preferentially used ENCODE data sets as a seed to map SNPs to DNA regulatory elements with putative function and used the results to associate these SNPs qualitatively (literature curation) and quantitatively (gene set enrichment in knowledge bases or network models) to predict downstream biomolecular mechanisms (Karczewski et al., Proc. Natl Acad. Sci. USA, 2013, 110: 9607-9612; Maurano et al., Science, 2012, 337:1190-1195; Schaub et al., Genome Res., 2012, 22: 1748-1759; Cheng et al., Genome Res., 2012, 22: 1658-1667; Gerstein et al., Nature, 2012, 489: 91-100; and Thurman et al., Nature, 2012, 489: 75-82). In contrast, approach presented in this example leverages ENCODE data to determine whether prior SNP-associated disease mechanism predictions share regulatory elements that might explain their convergent effects. New genome-wide regulatory annotations and quantitative trait loci analyses are now increasingly available such as those derived from chromatin accessibility and DNA methylation patterns of non-coding regions. Approaches relying on similarity of biological mechanisms could be systematically applied to these growing genomic data sets and further inform how common polymorphisms are involved in transcriptional or post-transcriptional mechanisms underlying the regulatory and cellular networks of disease progression.

This study highlights the significance of mechanistic similarities for uncovering additional interacting downstream effectors of intergenic SNPs predisposing individuals to the same disease. Identifying and understanding mechanisms of disease can not only inform biology but also provide insight in identifying candidate therapeutic targets. These results can be pursued for generating a comprehensive ‘roadmap’ of disease mechanisms revealed by downstream effectors of intergenic SNPs.

### Example 3—Materials and Methods Used in Above Examples

eQTL Association.

Two eQTL association data sets were acquired from SCAN-DB. The bulk of this analysis was done using an eQTL data set of the lymphoblastoid cell lines (Gamazon et al., Bioinformatics, 2010, 26: 259-262), which consisted of 4,189,682 associations between 833,004 distinct SNPs and 11,860 mRNAs at P [symbol] 10⁻⁴. Each SNP included for further study was matched to at least one eQTL transcript with a median of 2 transcripts per SNP (FIG. 12). The liver tissue eQTL dataset used for validation (FIG. 18) was comprised of 314,545 associations between 139,814 SNPs and 19,641 mRNAs at P [symbol] 10⁻⁵ (Innocenti et al., PLoS Genet., 2011, 7: el 002078). Trans effect was defined as 4 M bp from SNP to target mRNA based on the original definition (Duan et al., Am. J. Hum. Genet., 2008, 82: 1101-1113) and dbSNP build 138 (Sherry et al. Nuclic Acids Res., 2011, 29: 308-311) and refSeq (Pruitt et al., Nucleic Acids Res., 2007, 35: D61-D65) hg19 coordinates.

National Human Genome Research Institute GWAS Catalogue.

The dataset comprises 7,236 associations between 574 diseases/traits with 6,432 unique Lead SNPs (Hindorff et al., Proc. Natl Acad. Sci. USA, 2009, 106: 9362-9367).

dbSNP.

SNPs associated with human disease (National human genome research institute (NHGRI) GWAS catalogue) and mRNA expression (eQTL) were characterized as inter- or intragenic SNPs according to dbSNP (Build 138) definitions, which are based on RefSeq gene coordinates. Intragenic SNPs are located in regions whose boundaries extend 2 kb upstream of the transcription start site and 0.5 kb downstream of the terminator according to RefSeq (Pruitt et al., Nucleic Acids Res., 2007, 35: D61-D65). Intergenic SNPs are located between two intragenic regions (Sherry et al. Nuclic Acids Res., 2011,29:308-311).

Go Annotations.

GO annotations for human genes were retrieved from NCBI (Ashburner et al., Nat. Genet., 2000, 25: 25-29; and Berardini et al., Nucleic Acids Res., 2010, 38: D331-D335) and used to associate mRNA (eQTL) with molecular function (GO-MF) and biological process (GO-BP) terms. The database consisted of GO-MF and GO-BP annotations for 11,774 and 9,717 distinct genes (mRNAs), respectively.

STRING and Protein-Protein Interactions.

The STRING v9.1 database was used to determine PPIs among TFs (Franceschini et al., Nucleic Acids Res., 2013, 41: D808-D815). Only interactions between distinct TFs that scored [symbol] 0.9 were included in the enrichment analyses (FIG. 8).

ENCODE Data Set.

This data set provides DNA element annotations of the human genome based on various biochemical assays such as ChIP-seq, DNase-seq and RNA-seq (Dunham et al., Nature, 2012, 489: 57-74). Two types of ENCODE data were leveraged for the enrichment analyses: (i) combined data set of TF binding sites (TFBS-Clustered) comprising ChIP-seq of 148 TFs across 95 cell lines and (ii) three ChIA-PET data sets (Pol2, CTCF and ESR1) with data collected from cell lines, K562, HeLa, MCF-7, HCT-116 and NB4.

Prioritization of SNP Pairs.

2,358 SNPs (1,092 intergenic SNPs) associated with both disease risk and gene expression were included for a pairwise analysis. The HapMap CEU LD data set was used to determine Lead SNP pairs with LD of r²<0.8 or r²<0.01 (Altshuler et al., Nature, 2010, 467: 52-58). SNP pairs in strong LD (LD, r² [symbol] 0.8) were excluded from the study. Among the remaining pairs, inter-inter and inter-intra Lead SNP pairs (2,039,944) with at least one intergenic SNP were focused on. The three methods were employed based on a high-throughput computing system to prioritize biological mechanisms shared among SNP pairs: (i) mRNA overlap, (ii) molecular function similarity and (iii) biological process similarity. These prioritizations were controlled by permutation resampling of scale-free networks (Lee et al., PLoS Comput. Biol., 2010, 6: e1000730; and Barabasi et al., Science, 1999, 286: 509-512).

Computed Shared Mechanisms: mRNA Overlap and Semantic Biological Similarity of SNP Pairs.

Prioritization by mRNA overlap measured the number of shared mRNAs between two SNPs; typically, the number of shared mRNAs was directly related to mRNA overlap. Both non-statistical (any overlap) and statistical (prioritized by permutation resampling) types of mRNA overlap are reported. Prioritization by biological similarity was based on GO annotations of mRNA molecular functions or biological processes associated with the SNPs within each pair. Briefly, as every SNP within a pair could be associated with multiple mRNAs, and every mRNA could be associated with multiple GO terms, three steps were performed to impute biological similarly between two SNPs. First, the information theoretic semantic similarity (biological similarity) among GO terms (Lin et al., Proceedings 15^{th} International Conference on Machine Learning. 296-304 (Madison, Wis., USA, 1998)) was calculated as described in previous work (Tao et al., Bioinformatics, 2007, 23: i529-i538). The biological similarity of each pair of mRNAs was then computed within an SNP pair based on the average biological similarity of GO term pairs associated with the two mRNAs (Regan et al., JAMIA, 2012, 19: 306-316; and Pesquita et al., PLoS Comput. Biol., 2009, 5: e1000443). Finally, an algorithm was developed to impute the biological similarity of an SNP pair based on the average biological similarity of mRNAs associated with the two SNPs as the following ‘Equation (1)’

$$SNP\_ITS ( {s}_{1} , {s}_{2} ) = \frac{\begin{matrix}{\sum }_{{g}_{j} ∈ G ( {s}_{1} )} {max}_{{g}_{j} ∈ G ( {s}_{2} )} ( {GENE}_{ITS} ( {g}_{i} , {g}_{j} ) ) + \\ {\sum }_{{g}_{j} ∈ G ( {s}_{2} )} {max}_{{g}_{i} ∈ G ( {s}_{1} )} ( {GENE}_{ITS} ( {g}_{i} , {g}_{j} ) )\end{matrix}}{\vert G ( {s}_{1} ) \vert + \vert G ( {s}_{2} ) \vert} \qquad ( 1 )$$

where SNPₛ₁ was associated with a set of mRNAs G(ₛ₁), and |G(ₛ₁)| is the cardinality of the set G(ₛ₁), similarly for ₛ₂. The GENE_{ITS} is the biological similarity of two mRNAs (Regan et al., JAMIA, 2012, 19: 306-316; and Pesquita et al., PLoS Comput. Biol., 2009, 5: e1000443). The SNP_ITS provides a score that ranges from 0 to 1; a value of 1 indicated two SNPs with common GO-MFs or GO-BPs, and a value of 0 corresponded to two SNPs with unrelated GO-BPs or GO-MFs.

Permutation Resampling for Prioritization of Computed Shared Mechanisms.

The three prioritization methods were subjected to stringent statistical measurements to filter the relationship between two SNPs that could be observed by chance. In contrast to straightforward resampling methods, permutation resampling was performed with node-degree conservation on the entire eQTL association network (SNP-mRNA). Thus, the distinct probability of each SNP and mRNA could be controlled for, given original eQTL association network's topology. For each empirical permutation, the number of mRNAs associated with each SNP (SNP node degree) and the number of SNPs associated with each mRNA (mRNA node degree) conserved the same cardinality of connections as in the original eQTL data set. For each SNP pair, a P value was calculated as the proportion of empirical permutations (frequency among 100,000 times) with equal or greater strength of overlap or biological similarity than those observed. To adjust for multiplicity, the Benjamini-Hochberg FDR procedure was then used independently for each of the three prioritization methods using the p.adjust function in R software (http://www<dot>r-project<dot>org/). Prioritized SNP pairs were those yielding sufficient statistical significance using any of the prioritization methods.

Computations.

Approximately 20,000,000 core hours of high-throughput computations were conducted on the Beagle GLOBUS computing infrastructure housed in a Cray XE6 Supercomputer of the Computation Institute at the Argonne National Laboratory with peak performance of 151 teraflops generated by 17,424 compute cores (Foster et al., Int. J. High Perform. Comput. Appl., 1997, 11: 115-128; and Czajkowski, et al., Proceedings 10th IEEE International Symposium on High Performance Distributed Computing. 181-194 (San Francisco, Calif., USA, 2001).

Enrichment Analysis of Disease Mechanisms Among Prioritized SNP Pairs.

An enrichment analysis was performed to assess whether shared mechanisms (mRNA overlap, GO-MF/GO-BP similarity) were more likely found among SNP pairs related to the same disease than those across distinct diseases. Therefore, all SNP pairs were dichotomized into those associated with the same disease and those associated with distinct diseases based on the NHGRI GWAS catalogue. SNP pair enrichment was then performed by calculating ORs and P values according to the following contingency table: (same disease versus across-disease SNP pairs)×(prioritized versus non-prioritized SNP pairs) using Fisher's exact test in R. Enrichment tests were also performed at different P value cutoffs of eQTL associations ( [symbol] 10⁻⁴ to [symbol] 10⁻⁶) from which the number of mRNAs associated with each SNP served as a threshold for calculations ( [symbol] 1, [symbol] 3 and [symbol] 5 mRNAs per SNP).

Enrichment Analysis of Common Regulatory Properties Among Prioritized SNP pairs.

Pairs were prioritized according to computed shared mechanisms as described above. For each mechanism, it was determined whether prioritized SNP pairs were enriched in genomic regions with common regulatory properties: (i) same TF binding sites, (ii) interacting TFs and (iii) long-range chromatin interactions. Specifically, ENCODE data sets were leveraged to attribute DNA element annotation(s) to each SNP of the prioritized pairs, such as TF binding sites (ChIP-seq data) and/or anchored regions with long-range interactions (ChIA-PET) data. The regulatory annotation of the Lead SNPs to SNPs was extended in strong LD (r² [symbol] 0.8) with each Lead SNP of a pair. RegulomeDB (Boyle et al., Genome Res., 2012, 22: 1790-1797) was used to determine Lead SNPs in strong LD (LD SNPs; r² [symbol] 0.8) for which ENCODE-derived functional annotations were available. The first enrichment analysis assessed whether prioritized SNP pairs are more likely than non-prioritized pairs to be enriched in regions sharing common regulatory properties using the following contingency table: (same regulatory properties versus different regulatory property of Lead SNP pairs)×(prioritized versus non-prioritized Lead SNP pairs). The second enrichment analysis was performed to determine whether prioritized SNP pairs related to the same disease are more likely to share common regulatory properties than those associated with distinct diseases using the contingency table: (same disease and regulatory properties versus distinct diseases and/or different regulatory property Lead SNP pairs)×(prioritized versus non-prioritized Lead SNP pairs). A control was included in which SNP pairs were calculated from every possible combination of SNPs with an eQTL association. All Lead SNP pairs derived from the NHGRI GWAS catalogue were used as the background, and enrichment analyses were performed on SNP pairs derived from eQTL associations with P [symbol] 10⁻⁴. Bar graphs were generated using Prism v.6 (GraphPad Software Inc, La Jolla, Calif. USA).

GWAS-Based Detection of Epistatic Effects Among Mechanism-Anchored Prioritized Lead SNP Pairs.

Prioritized intergenic Lead SNP pairs associated with bladder cancer (BC) or Alzheimer's disease (AD) were considered for genetic interactions in GWAS (BC: rs9642880-rs1495741 and rs8102137-rs1014971; AD: rs7081208-rs9331888, rs17511627-rs9331888, rs3818361-rs4509693, rs381836-rs7081208, rs4509693-rs753129 and rs4509693-rs6656401). The multifactor dimensionality reduction machine-learning method (Ritchie et al., Am. J. Hum. Genet., 2001, 69: 138-147) was first applied for modelling the joint effects of the Lead SNP pairs. The multifactor dimensionality reduction approach was implemented using 10-fold cross-validation for estimating generalizability, followed by a 1,000-fold permutation test to determine statistical significance and to address multiple testing issues. In addition, the explicit test of epistasis was applied, which uses permutation testing to determine statistical significance of interaction effects while holding the main effects constant (Greene et al., Pac. Symp. Biocomput., 2010, 15: 327-336). An entropy-based information gain approach (Hsieh et al., Genomics, 2011, 97: 77-85; and Moore et al., J. Theor. Biol., 2006, 241: 252-261) was used as an additional method for interpreting the statistical pattern of epistasis. The BC GWAS included 3,532 cases and 5,119 controls from the Cancer Genetic Markers of Susceptibility for Bladder Cancer study (Rothman et al., Nat. Genet., 2010, 42: 978-984), which is available from dbGaP (accession: phs000346.v1.p1). The AD GWAS included 529 cases with mild cognitive impairment or AD and 204 controls from Phase I of the Alzheimer's Disease Neuroimaging Initiative (Shen et al., Neuroimage, 2010, 53: 1051-1063), also available from dbGaP (accession phs000219.v1.p1).

PheWAS Identification of Genetic Interactions Among Mechanism-Anchored Prioritized Lead SNP Pairs.

Each RA-associated prioritized inter-inter and inter-intra Lead SNP pair was considered for SNP-SNP interactions using a data set selected from the Vanderbilt University EMR-linked DNA biobank (BioVU) (Roden et al., Clin. Pharmacol. Ther., 2008, 84: 362-369). To identify RA case-controls cohort from the EHR, previously developed PheWAS case-control definitions for RA were utilized that can reproduce known genetic associations (Denny et al., Nat. Biotechnol., 2013, 31: 1102-1110; and Denny et al., Bioinformatics, 2010, 26: 1205-1210). From a population of approximately 36,000 individuals with extant Illumina Human Exome chip genotype data in the deidentified Vanderbilt University clinical data warehouse linked to BioVU (Roden et al., Clin. Pharmacol. Ther., 2008, 84: 362-369), 1,115 RA cases and 24,169 controls were identified. Cases had at least two ICD-9-CM billing codes specific to RA (714.0, 714.1, 714.2 or 714.81) on different days. Controls were selected among patients with no RA or related diagnoses (e.g., juvenile idiopathic arthritis, psoriatic arthritis) reported in their billing history according to the PheWAS approach. Individuals with RA noted on a single day were excluded, as these cases often have poorer positive predictive value.

For each patient, DNA and genotype data was previously extracted for 233,605 SNPs with <5% missing data using the Illumina Human Exome 12v.1 array. Genotypes were quality controlled for call rate (495%), minor-allele frequency (41%) and identity by descent to remove related individuals. Among these genotyped SNPs, three prioritized Lead SNP pairs (involving SNPs ‘alleles’ rs6457617-‘T/C’, rs9272219-‘T/G’ and rs9268853-‘C/T’) associated with RA were available for calculations. Only individuals identified from European ancestry by Structure (Pritchard et al., Genetics, 2000, 155: 945-959) were used in the analysis, resulting in 29,731 individuals before case and control selection. All association analyses were completed with PLINK v1.07 (Purcell et al., Am. J. Hum. Genet., 2007, 81: 559-575) using logistic regression adjusted for age and sex and assuming an additive genetic model. Interaction analyses were also performed on the second SNP of each pair and included an SNP-SNP interaction term (RORᵢ). Interactions between specific alleles of Lead SNP pairs were analyzed by Fisher's exact test. ORs of allelic combination effects associated with RA and their 95% confidence intervals were reported using PLINK v1.07. Submission to dbGaP of RA genotypes and phenotypes of the present PheWAS study is in process (Mailman et al., Nat. Genet., 2007, 39: 1181-1186).

Network of Predicted Mechanisms Shared by Disease-Associated Prioritized Lead SNP Pairs.

On the basis of the disease-specific results of this study, a global network of functional annotations was constructed that comprises biological molecules and their relationships across the three prioritization methods (SNP-mRNA eQTL, prioritized SNP-SNP association and computed SNP-GO-SNP association). Disease-specific networks curated to highlight overlap and similarity of mechanisms found among prioritized Lead prioritized SNP pairs associated with RA. Networks were visualized using Cytoscape (Shannon et al., Genome Res., 2003, 13: 2498-2504).

Calculation of SNP Similarity.

This calculation requires three steps (A1, A2, and A3 described below).

A1. Information Theoretical Semantic Similarity of Two Gene Ontology (GO) Terms ITS).

Lin's method (Lin et al., Proceedings 15^{th} International Conference on Machine Learning. 296-304 (Madison, Wis., USA, 1998)) was used to calculate the ITS of two GO terms, t₁, t₂. This computes their similarity regarding ontology topology (e.g., two biological processes or two molecular function terms). It was defined (Equation 2) as the ratio of the information content (ic) of the minimal common ancestor mca of the two terms (ic(ms,t₁,t₂)) over the average information content of these two terms ([ic(t₁)+ic(t₂)]/2).

$$ITS ( {t}_{1} , {t}_{2} ) = \frac{2 * ic ( mca ( {t}_{1} , {t}_{2} ) )}{ic ( {t}_{1} ) + ic ( {t}_{2} )} \qquad ( 2 )$$

The information content ic of a GO term t was defined by the negative logarithm of the probability calculated as the ratio of (i) the number of terms in the ontology subgraph rooted at term t: (Ψ(t)); divided by (ii) the total number of terms in the Gene Ontology category rooted at the topmost ancestor r: (Ψ(r)) (Equation 3)

$$ic ( t ) = - log ( \frac{\vert Ψ ( t ) \vert}{Ψ ( r )} ) \qquad ( 3 )$$

where Ψ(t) represents the subgraph of GO terms rooted at GO term t, and |Ψ(t)| is the cardinality of the subgraph. This measure of ITS (Equation 2) value is thus scaled between 0 and 1, where 1 is a complete overlap of subgraph between two GO terms. For more details and examples, please refer to previous papers (Tao et al., Bioinformatics, 2007, 23: i529-i538 Li et al. JAMIA, 2012, 19: 295-305; and Regan et al., JAMIA, 2012, 19: 306-316).

A2. Information Theoretical Similarity of Two mRNAs (GENE_ITS).

A conventional method was used to calculate the similarity (GENE_ITS) of two mRNAs (Equation 4). For the purpose of this calculation, the genes from which each mRNA was transcribed were used and thus do not mention mRNA in the calculations. In GO, each gene may be annotated to one or multiple Gene Ontology terms, noted as the “Set of GO terms of gene x” or simply “T(gₓ)”. The similarity between gene 1 (g1) and gene 2 (g2) is measured using the semantic similarity (Equation 2) between the set of GO terms associated to gene 1 (T(g1)) and those associated to gene 2 (T(g2)). Precisely, for a specific GO term (ti) associated to gene 1, its GO similarity score (ITS(ti, tj)) was calculated to every GO term (tj) associated to gene 2 (tj∈T(g2)) and retain the maximum value among them. This is repeated for each GO term associated to gene 1. Next, the converse was calculated for each GO term associated to gene 2. Then, the average of all these maximum similarity scores was calculated to obtain the similarity of the two genes, noted as GENE_ITS(g1,g2). The GENE_ITS was formally denoted as follows:

$$GENE\_ITS ( {g}_{1} , {g}_{2} ) = \frac{{\sum }_{{t}_{i} ∈ T ( {g}_{1} )} {max}_{{t}_{j} ∈ T ( {g}_{2} )} ( ITS ( {t}_{i} , {t}_{j} ) ) + {\sum }_{{t}_{j} ∈ T ( {g}_{2} )} {max}_{{t}_{i} ∈ T ( {g}_{1} )} ( ITS ( {t}_{i} , {t}_{j} ) )}{\vert T ( {g}_{1} ) \vert + \vert T ( {g}_{2} ) \vert} \qquad ( 4 )$$

where gene g1 was annotated to a set of GO terms, T(g1), and |T(g1)|, is the cardinality of the set T(g1). The GENE_ITS provides a score that ranges from 0 to 1, where GENE_ITS of 0 corresponds to two genes with no similar GO annotations and GENE_ITS of 1 corresponds to two genes with the same GO annotations.

A3. Novel Semantic Biological Similarity of Two SNPs Using eQTL Associations (SNP_ITS).

A new method to calculate the biological similarity of two SNPs using their SNPmRNA eQTL associations (or SNP_ITS for short) (Equation 5). The similarity between SNP 1 (s1) and SNP 2 (s2) is measured using the semantic similarity (Equation 4) between the set of mRNAs associated to SNP 1 (G(s2)) and those associated to SNP 2 (G(s2)). Precisely, for a specific mRNA (gi) associated to SNP 1, its similarity score (GENE_ITS(gi, gj)) was calculated to every mRNA (gj) associated to SNP 2 (gj∈G(s2)), and retain the maximum value among them. This is repeated for each mRNA associated to SNP 1. Then the converse is calculated for each mRNA associated to SNP 2. Then, the average of all the maximum similarity scores was calculated to obtain the similarity of the two SNPs, noted as SNP_ITS (s1, s2). The SNP_ITS of two SNPs was formalized as follows:

$$SNP\_ITS ( {s}_{1} , {s}_{2} ) = \frac{\begin{matrix}{\sum }_{{g}_{i} ∈ G ( {s}_{1} )} {max}_{{g}_{j} ∈ G ( {s}_{2} )} ( GENE\_ITS ( {g}_{i} , {g}_{j} ) ) + \\ {\sum }_{{g}_{j} ∈ G ( {s}_{2} )} {max}_{{g}_{i} ∈ G ( {s}_{1} )} ( GENE\_ITS ( {g}_{i} , {g}_{j} ) )\end{matrix}}{\vert G ( {s}_{1} ) \vert + \vert G ( {s}_{2} ) \vert} \qquad ( 5 )$$

where SNP s1 was associated to a set of mRNAs G(s1), and |G(s1)| is the cardinality of the set G(s1), similarly for s2. The SNP_ITS provides a score that ranges from 0 to 1, where SNP_ITS of 0 corresponds to two SNPs with neither similar mRNA annotations nor similar GO annotations. SNP_ITS of 1 corresponds to two SNPs with the same mRNA annotations or the same number of mRNAs each having the same GO annotations.

Permutation Methods.

eQTL associations were selected by cutoffs of p-values between 10⁻⁴ and 10⁻⁶ (p≤10⁻⁴, p≤5×10⁻⁵, p≤10⁻, and p≤10⁻⁶) and SNP node degree (ND) threshold between 1 and 5 (ND≥1, ND≥3, and ND≥5). Each eQTL dataset was regarded as a bipartite network consisting of SNP and mRNAs. A conservative permutation resampling that keeps the node degree of each specific SNP and specific mRNA constant (as observed) was conducted for each dataset (100,000 permutations). By this means, the permutation resampling matched the probability of a SNP or mRNA connected in the bipartite (from 1/# SNPs to 1 for SNPs and from 1/# mRNAs to 1 for mRNAs). Three types of empirical permutation were conducted: mRNA overlap, GO biological process, and molecular functions. The one for mRNA overlap was directly permutated from the eQTL dataset with respect to a p-value cutoff and a node degree threshold. For GO biological process and molecular functions, the genes without respective GO annotations were filtered out before permutation, and the subsequent calculation of biological semantic similarity and significance were based on the filtered ones, by which potential biases from incomplete annotation could be controlled. The same set of permutations was used for calculation of GO biological process similarity of SNP pairs and specific overlapping GO biological process terms in the SNP pairs. The same rule was applied to calculations for GO molecular function. The permutations were conducted in supercomputers (specifically beagle) or clusters using MPI parallel computing.

Q-Q Plot Analysis.

Quantile-quantile (Q-Q) plot was employed to show the distribution difference of SNP pair measures between the pairs of SNPs associated with the same complex diseases and those with different diseases. Two types of measures were conducted: FDR of mRNA overlap and FDR of mRNA similarity (GO-BP). To show the relative trend for SNP pairs derived from different eQTL strengths, multiple Q-Q plot curves were assembled in one panel, such as different p-value cutoffs of eQTL associations (p≤10⁻⁴, p≤10⁻, and p≤10⁻⁶), and different SNP node degree (ND) thresholds (ND≥1, ND≥3, and ND≤5). The Q-Q plot curves derived by multiple eQTL cutoffs were generated by the “qqplot” function in R software and the output of the function were extracted and plotted with customized shapes and colors to distinguish these curves. Since the p-values of SNP pairs were derived from empirical permutations (e.g. 100,000 times), they were truncated at the minimal observable p-values (10⁻⁵) and manually set as 90% of that (e.g. 9*10⁻⁶), with corresponding FDR truncated accordingly. The axes of the figure, each for one group of SNP pairs, were shown in negative log₁₀ scale (−log₁₀ FDR) to better visualize the pattern. In addition, Mann-Whitney U test (“wilcox.test” function in R) was performed to evaluate the overall mean between the two groups of SNP pairs of each curve, using FDR values directly rather than negative log values. The p-value result of each U-test was shown along with its correspondingly Q-Q plot curve.

Disease Network: Assessment of the GO Term Overlaps Significance within a SNP Pair.

This method focuses on assessing the significance of an overlap of GO terms within a SNP pair (SNPGO-SNP triplet). It allows explaining which functional relationships are more likely to relate two SNPs of prioritized SNP pair. The method requires four steps. First, all Lead SNPs in the studied SNP pairs were related to mRNA by eQTL associations from the SCAN database. Second, each of these associated mRNAs was related to each of their corresponding GO terms using the Gene Ontology Annotations (molecular functions (GO-MF) biological processes (GO-BP)). The fully-derived GO annotation table was utilized, where each GO terms is directly associated to mRNAs and all mRNA associations to the ancestral GO terms. GO terms are organized in a hierarchical structure (directed acyclic graph). Third, GO terms were assigned to Lead SNPs via their respective mRNAs. For each SNP of the Lead SNP pair, a list of associated GO terms is calculated. Finally, from these associations, overlapping GO terms can be identified between two Lead SNPs of the prioritized Lead SNP pair. Then, the statistical significance (p-value) of those overlapping GO terms was imputed using empirical permutation resamplings (100,000 times) of SNP-mRNA associations based on eQTLs (eQTL p-value cutoff≤10⁻⁴ for the reported results). Under these permutations, every mRNA is associated to the same number of SNPs and each SNP to the same number of mRNAs (constant node degree, constant number of total mRNA-SNP associations; see above section). However, the association of SNP-GO terms differs from the observed set through resampling. False Discovery Rate (FDR) was used to adjust for multiplicity. These calculations were performed separately for GO-BPs and GO-MFs to derive prioritized Lead SNP pairs and then combined. Each significant SNP-GO-SNP triplet (FDR<0.05) was considered as prioritized and therefore, as a putative shared mechanism for the prioritized SNP pair.

### Example 4—Convergent Biomolecular Mechanisms Between Bipolar Disorder and Schizophrenia Revealed by Multiscale Integration and Modeling of GWAS, eQTL, Gene Ontology, ChIP-Seq, and Protein Interactions

Schizophrenia and bipolar disorder are highly comorbid conditions with strong evidence for shared genetic etiology underlying much of the heritable disease risk. High dimensional data was computationally modeled from genome-wide association studies (GWAS), expression Quantitative Trait Loci (eQTL), gene product functional annotations (GO), and high-throughput locus functional annotations (ChIP-Seq, ENCODE project) to explore biological similarity and convergence between all possible pairs of GWAS polymorphisms associated with either or both diseases (151 teraflops). This strategy provided high confidence and recurrent biological signals from intergenic SNPs as well as those within candidate gene regions.

SNP pairs where both polymorphisms had been mapped to schizophrenia alone reproduced a strong antigen presenting and immune/inflammatory process signal driven by the Major Histocompatibility Locus (MHC: Chr 6: 29 Mb-33 Mb) that was not found in bipolar disorder. However, most other biological convergence was found between the two diseases, or for SNP pairs that were mapped in comorbid samples. This underlines the molecular and mechanistic similarity for bipolar disease and schizophrenia even in the absence of GWAS positional overlap. Three hundred and eighty-four (384) SNP-SNP pairs (involving 66 unique SNPs) had statistically significant evidence that they each influenced a common biological process or were each bound by the same transcription factor. Sixteen pairs of SNPs were found to regulate the expression of at least one of the same mRNA transcripts via eQTL, including four SNP-SNP pairs mapped across two different chromosomes and acting in trans (regulating expression of RAB11FIP4, MED29 and HIST1H2BN).

Common biological process annotations to both diseases included additional immune responses, chromatin architecture and histone biology, with overrepresentation of binding by the transcriptional repressor CTCF and chromatin remodeling factor FOXA1. The multiomic approach used in this case study is extensible to other diseases or syndromes, with a data management system currently being built to mine preprocessed results from 400 additional GWAS traits.

### Example 5—Identify Environmental and Lifestyle Stressors (ELSs) Contributing to Excess Comorbidity within Diverse Populations Through Analysis of Electronic Medical Records (EMR)

With the present invention, the shared genetic mechanisms between diseases can serve as candidate biomarkers of disproportionate clinical comorbidities that become further disrupted by ELSs associated with health disparities.

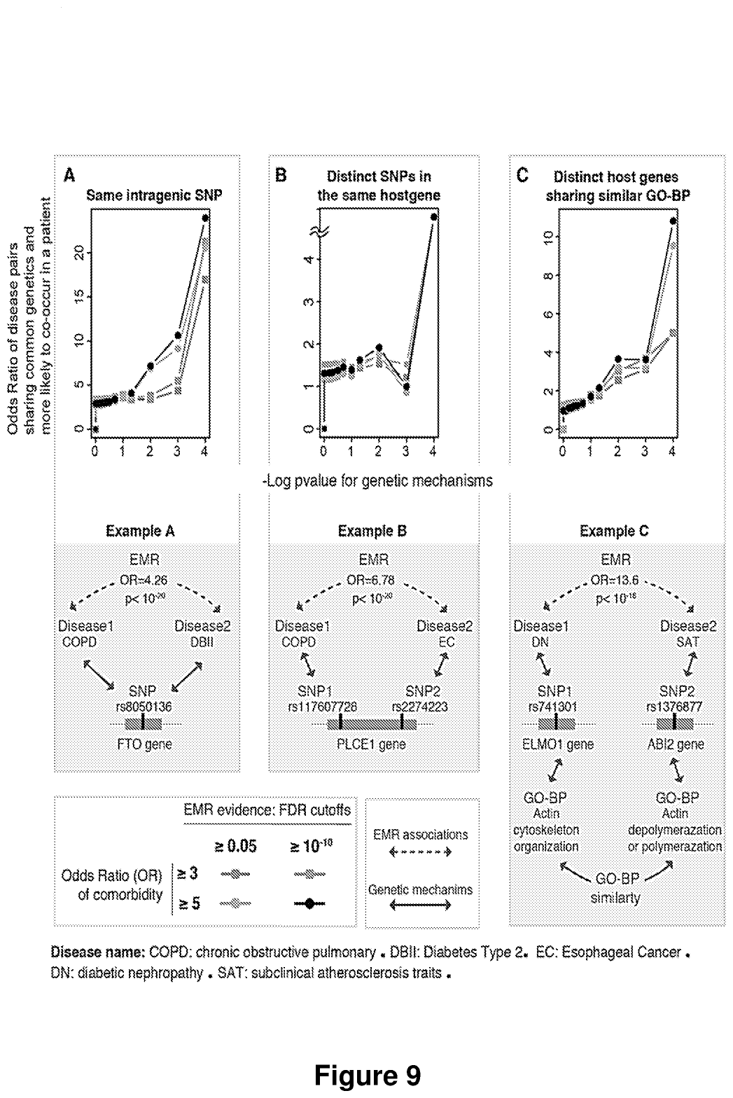

As illustrated in FIG. 9, diseases that are more likely to co-occur in a patient are also more likely to share a common genetic mechanism as shown in this big data study. Using the diseases and polymorphisms described in the National Human Genome Research Institute (NHGRI) compendium of GWAS (Welter et al., Nucleic Acids Research, 2014, 42: D1001-D1006), statistically significant disease pairs were identified that likely share a common genetic mechanism via: (A) one or more common SNPs as shown in Panel A; (B) excess SNPs more likely sharing the same host gene location in the genome (Panel B); or (Panel C) for which the SNPs were located in distinct, yet functionally similar, host genes (Panel C).

In addition, using contingency table statistics, 5,754 pairs of diseases with disproportionate comorbidity were identified in the HCUP clinical dataset (Table 2) at false discovery rate <5% (FDR) among 16,701 combinations tested.

TABLE 2

| | Dataset | |
|---|---|---|
| | Healthcare Cost & | UA Clinical Data Warehouse (Integrated |
| | Utilization Project (HCUP) | Banner-UAMC + UA-HPQ) |
| Aim | Comorbidity-Disparity Discovery | External validation of identified |
| | | comorbidity-disparity |
| Key disparity | age, sex, race, patient/hospital | UA-HPQ contains 45 KDFs & contextual |
| factors & | zip code, homelessness, median | variables (e.g. those mentioned in HCUP |
| contextual | income in patient's zip code, and | and daily activities, substance abuse of |
| variables | urban-rural status, etc | tobacco/alcohol/street drug abuse) |
| Location/Sector | California (2011)/Public Sector | Tucson, AZ/Private Sector Dataset |
| | Dataset | |
| Total | 6,987,483 | 1,927,672 (Banner-UAMC) ∩ 239,460 |
| Population | | (UA-HPQ) = 59,544 subjects |
| Hispanic (%) | 28% | 40% |

The legend at the bottom left of FIG. 9 shows different cutoffs of Odds Ratio (OR) and FDRs for these clinical dataset analyses (EMR=electronic medical record).

The enrichment of disease pairs were then calculated at (Vertical axes, Panels A-C; n=16,701) identified with (i) shared molecular mechanisms in GWAS as well as (ii) found in excess comorbidity in the HCUP clinical dataset. Finally, the concordance between the specific molecular mechanisms and the clinical co-occurrences are provided at the bottom of each Panel. Examples A, B and C in FIG. 9 illustrate that each of the 16,071 disease pairs investigates are prioritized by genetic mechanisms (bottom full arrows) and also by enrichment of co-occurrences in EMR patients (top dotted lines and pvalues; Fisher's Exact Test).

Methods.

Intragenic SNPs are defined as within the same gene locus, 2 kb upstream, or 0.5 kb downstream (see dbSNP, Sherry et al. Nuclic Acids Res., 2011, 29: 308-311). Gene similarities of Panel C are identified via regularly updated software (Tao et al., Bioinformatics, 2007, 23: i529-i538) for information theoretic similarity calculations using Gene Ontology (Berardini et al., Nucleic Acids Res., 2010, 38: D331-D335) and an additional software recently designed for diseases similarities (Li et al. JAMIA, 2012, 19: 295-305). All of the disease pairs were then assessed for co-occurrence in the EMR (Fisher's Exact Test). Datasets: SNPs from NHGRI GWAS catalog (6432 SNPs associated with 574 distinct diseases downloaded in 2012²). Expression Quantitative Trait Loci (eQTL) from SCAN-DB (Gamazon et al., Bioinformatics, 2010, 26: 259-262); Host similarity: 129 curated diseases; the HCUP EMR dataset: 6,987,475 patients in year 2011.

### Example 6—Convergent Downstream Candidate Mechanisms of Independent Intergenic Polymorphisms Between Co-Classified Diseases Implicate Epistasis Among Noncoding Elements

Eighty percent of DNA outside protein coding regions has been shown to be biochemically functional by the ENCODE project, enabling studies of their interactions. Such studies have since explored how convergent downstream mechanisms arise from independent genetic risks of one complex disease. However, the cross-talk and epistasis between intergenic risks associated with distinct complex diseases have not been comprehensively characterized. Recent integrative genomic analysis unveiled downstream biological effectors of disease-specific polymorphisms buried in intergenic regions that were validated in terms of their genetic synergy and antagonism in distinct GWAS. This approach was extend to characterize convergent downstream candidate mechanisms of distinct intergenic SNPs across distinct diseases within the same clinical classification.

A multipartite network was constructed consisting of 467 diseases organized in 15 classes, 2,358 disease-associated SNPs (NHGRI GWAS catalog), 6,301 SNP-associated mRNAs (expression Quantitative Trait Loci), and mRNA annotations to 4,538 Gene Ontology (GO) biomolecular mechanisms. Functional similarity between two SNPs (similar SNP pairs) is imputed using a nested information theoretic distance model for which p-values are assigned by conservative scale-free permutation of network edges without replacement (node degrees constant). At FDR≤5%, 3,870 intergenic SNP pairs were prioritized, among which 755 pairs are associated with distinct diseases sharing the same disease class, implicating 167 intergenic SNPs, 14 classes, 230 mRNAs, and 134 GO terms. Co-classified SNP pairs were more likely to be prioritized as compared to those of distinct classes confirming a noncoding genetic underpinning to clinical classification (odds ratio ˜3.8; p≤10⁻²⁵). The prioritized pairs were also enriched in regions bound to the same/interacting transcription factors and/or interacting in long-range chromatin interactions suggestive of epistasis (odds ratio ˜2,500; p≤10⁻²⁵). This prioritized network implicates complex epistasis between intergenic polymorphisms of co-classified diseases and offers a roadmap for a novel therapeutic paradigm: repositioning medications that target proteins within downstream mechanisms of intergenic disease-associated SNPs.

Overview.

Human diseases can be classified via multiple criteria: cell type, tissue, organ, system, topological body region, pathophysiological, epidemiological characteristics, and etiological causes. Thus, in clinical classification of diseases, genetic disorders have conventionally relegated to a subset of the classification pertaining to its etiology. The advent of genomic assays now offers the opportunity to utilize unbiasedly a broad number of molecules of life to redefine the architecture of clinical classifications.

For example, cancers pertaining to distinct organ and cell types have been shown to share common somatic mutations or transcriptomes and sometimes respond to the same therapy in spite of their distinct conventional classification, suggesting a new systems oncology etiology to cancer pathophysiology (Inaki et al., Trends in Genetics, 2012, 28(11): 550-559). It has been previously shown that the miRNome of tumors classify the primary cancers by organ of origin as expected, while their paired metastases remarkably classify according to their progression (oligometastatic vs. polymetastatic) regardless of the primary site and metastatic site (Lussier et al., PloS one, 2011, 6(12): e28650). Recently, Genome-Wide Association Studies (GWAS) have implicated the same polymorphisms to distinct diseases of the same clinical class (e.g., cardiovascular system). Many distinct autoimmune diseases are found to have the same polymorphisms relating to the major histocompatibility complex region of chromosome 6, along with some other chromosome regions involving signaling in immune response (e.g., cytokine, interleukin, and interferon) (Seldin et al., Journal of autoimmunity, 2015, 64: 1-12; and Zenewicz et al., Cell, 2010, 140(6): 791-797). These same polymorphisms have also been associated with distinct traits of the metabolic syndrome (Vattikuti et al., PLoS genetics, 2012, 8(3): e1002637).

In addition to studying each disease class separately, studies have also been conducted at a system level to unveil mechanisms that link individual diseases to a disease class. A disease class is likely to be driven by common genes and even common biological sub-networks, thus rendering a cluster structure or modularity in the biological network that separates it from other classes (Maurano et al., Science, 2012, 337(6099): 1190-1195). The modularity for disease classes has been observed in various types of molecular networks based on their risks identified in shared intragenic regions, including disease-gene networks, drug-target networks, transcription factor networks, and protein-protein interaction networks (Lee et al., Summit on Translational Bioinformatics, 2010, 31; Maurano et al., Science, 2012, 337(6099): 1190-1195; Goh et al., Proc. Natl Acad. Sci. USA, 2007, 104(21): 8685-8690; Jiang et al., FEBS letters, 2008, 582(17): 2549-2554; Bulik-Sullivan et al., Nature genetics, 2015, 47(11): 1236-1241; Yildirim et al., Nature biotechnology, 2007, 25(10): 1119; Karczewski et al., Proc. Natl Acad. Sci. USA, 2013, 110(23): 9607-9612; and Sam et al., Pacific Symposium on Biocomputing, 2007, 76).

Ohn broadened the similarity between diseases by looking into correlated polymorphisms by GWAS p-values (Ohn et al., JAMIA, 2017: ocx026). In addition, two studies leveraged trans-expression Quantitative Trait Loci (eQTL) analyses studies respectively limited to the immune systems and node-degree properties (Fehrmann et al., PLoS genetics, 2011, 7(8): el 002197; and Li et al., Journal of Biomedical Informatics, 2015, 58: 226-234). Traditional genetic-interaction studies such as PLINK and BOOST, as well as recent integrative functional studies on non-coding disease variants, such as GWAS3D and CEPID may also provide insight into how distinct diseases of the same disease class co-classify together (Purcell et al., The American Journal of Human Genetics, 2007, 81: 559-575; Wan et al., The American Journal of Human Genetics, 2010, 87: 325-34; ENCODE Project Consortium, Nature, 2012, 489: 57; Lee et al., PLoS Genetics, 2012, 8: e1002998; Li et al., Nucleic Acids Research, 2013, 41: W150-W158; and Li et al., Genome biology, 2017, 18: 52). In spite of the genetic, genomic, and biological network studies generally conducted for specific disease classes, the biological mechanisms of the majority of disease-associated intergenic polymorphisms remain obscure as well as their contribution to explaining these risks at the disease class level.

It has been recently reported that downstream functional effects of distinct intergenic single-nucleotide polymorphisms (SNP pairs) associated with the same complex disease are likely to converge at some levels of biology such as sharing downstream transcripts or regulating functionally similar biological pathways or processes (Li et al., NPJ genomic medicine, 2016, 1: 16006). Research groups have confirmed genetic synergy or antagonism between the top prioritized convergent intergenic SNP-pairs in a GWAS of Alzheimer's and a Phenome-Wide Association Study (PheWAS) of rheumatoid arthritis (Li et al., NPJ genomic medicine, 2016, 1: 16006). However, this study did not address the convergent mechanism of SNP pairs between distinct diseases associated with the same clinical classification (co-classified).

Here, the downstream functional similarity between two SNPs (similar SNP pairs) is imputed using a multiscale information theoretic distance model for which p-values are assigned by conservative permutation resampling of network edges without replacement (node degrees constant). It is hypothesized that this approach could be approached to identify downstream mechanisms of intergenic SNPs with distinct co-classified diseases, by integrating the classification information of the NHGRI diseases/traits and reanalyzing the results, to infer the noncoding genetic architecture of disease classes, which has implications for drug repositioning and mitigation of risks for multiple diseases within the same class.

Main Datasets.

Lead SNPs (SNPs investigated in GWAS) were surveyed from two datasets, the National Human Genome Research Institute (NHGRI) GWAS catalog and the eQTL association dataset named SNP and Copy Number Variant Annotation (SCAN) database (Welter et al., Nucleic acids research, 2013, 42(D1): D1001-D1006; and Gamazon et al., Bioinformatics, 2009, 26(2): 259-262). The NHGRI GWAS catalog provides a comprehensive resource by systematically cataloging and summarizing the key characteristics of reproducible trait/disease associated SNPs from currently published GWAS (Welter et al., Nucleic acids research, 2013, 42(D1): D1001-D1006). The NHGRI GWAS catalog comprises 7,236 associations between 574 diseases/traits and 6,432 distinct SNPs (Jun. 7, 2012). The SCAN database contains 4,189,682 eQTL associations between 833,004 distinct SNPs and 11,860 mRNA at P≤10⁻⁴ from lymphoblastic cell lines. The integration of these two datasets yields 2,358 Lead SNPs in common (1,092 intergenic SNPs), along with their traits/diseases and mRNA information. The 574 NHGRI diseases/traits were classified into 15 organ and clinical system disease classes according to Maurano et al. (Science, 2012, 337(6099): 1190-1195) along with curation.

A pairwise analysis was conducted on all possible combinations of two Lead SNPs inherited in distinct haplotypes (pairs of SNPs in strong linkage disequilibrium (LD) were removed from the study). The HapMap CEU LD dataset was used to determine LD level and the exclusion criterion of r²≥0.8 (Gibbs et al., The international HapMap project, 2003). Since the major interest is in intergenic variants (i.e., located between genes), the pairs in which both SNPs are intragenic (i.e., located within genes) were also excluded. The definition of “intergenic” and “intragenic” are derived from dbSNP (Build 138 on Feb. 21, 2014) (Sherry et al., Nucleic acids research, 2001, 29(1): 308-311), which considers a SNP in a gene region to be intragenic if it is within 2 kb upstream (5′ side) or 0.5 kb downstream (3′ side) of that gene. ˜2.8 million pairwise combinations were derived from these Lead SNPs with r²≥0.8, associated with 467 diseases, 6,301 mRNAs, 1,635 molecular functions (MF), and 2,903 biological processes (BP). Among them, 1,977,927 pairs contain at least one intergenic SNP (named as intergenic Lead SNP pairs): 595,053 intergenic-intragenic and 1,382,874 intergenic-intergenic. 800,438 pairs are intragenic-intragenic. Among the intergenic Lead SNP pairs, 211,808 are associated with same disease classes (i.e., each SNP in one pair is associated with a specific disease class) while 1,766,119 are associated with distinct ones.

Calculation of SNP Similarity.

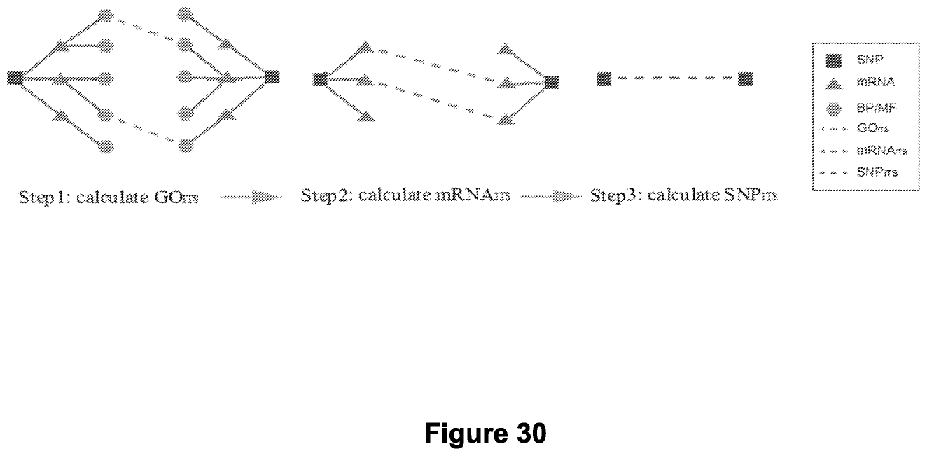

The prioritization process was applied to the intergenic Lead SNP pairs based on their convergence of eQTL-associated biological mechanisms. Three approaches were exploited to determine such shared (convergent) mechanisms: (1) eQTL-associated mRNA overlap, (2) molecular function (MF) similarity of eQTL-associated mRNA, and (3) biological process (BP) similarity. MFs and BPs of each mRNA associated with a SNP from gene ontology (GO) annotations were extracted (Ashburner et al., Nature genetics, 2000, 25(1): 25-29; and Consortium et al., Nucleic acids research, 2010, 38(suppl 1): D331-D335) to calculate the similarity of a SNP pair (Li et al., NPJ genomic medicine, 2016, 1: 16006) (see Table 3 and FIG. 30).

TABLE 3
Nested calculations (3 steps)

1. Calculate the Information Theoretic Similarity (ITS) between two GO terms (GO_{ITS})
associated with the two SNPs through mRNAs using Lin's method (Lin, D, An information-
theoretic definition of similarity. in Icml. 1998; and Tao et al., Bioinformatics, 2007. 23(13):
i529-i538).
2. Based on GO_{ITS}, calculate the information theoretic similarity between two distinct mRNAs
(mRNA_{ITS}), each associated with a SNP within a SNP Pair, using Tao's approach (Tao et al.,
Bioinformatics, 2007, 23(13): i529-i538; Regan et al., JAMIA, 2012, 19(2): 306-316; and Li
et al., J. Am. Med. Inform. Assoc., 2012, 19: 295-305)
3. Determine the semantic biological similarity between two SNPs (SNP_{ITS}) within a SNP pair
using the (mRNA_{ITS}) of pairs of mRNAs associated with the two SNPs respectively, using
Li's nested ITS approach as recently published (Li et al., NPJ genomic medicine, 2016, 1:
16006). The SNP_{ITS} values range from 0 to 1, with 0 corresponding to no similar downstream
effects and 1 corresponding to identical downstream effects (e.g., either the same mRNAs or
distinct mRNAs with the equivalent GO terms). The similarity measurement between SNPs
can capture relationships between SNPs including the ones without any common mRNAs in
their eQTL associations.

Network Permutation to Establish the p-Values for Observed mRNA Overlap and ITS Scores Between Two SNPs.

To determine the statistical significance of imputed biologically convergent mechanisms of SNP pairs, permutation of the eQTL network was conducted for mRNA overlap, molecular function similarity, and biological process, separately. The eQTL associations of SNPs not known to be associated with any diseases were also included to create a null distribution of SNP mRNA overlap (statistical mRNA overlap) and ITS. When examining the significance of each of the three mechanisms, the original node degree (ND) of each specific SNP and each specific mRNA were controlled. Specifically, the number of mRNAs associating with one SNP the same, or vice versa, was kept during the resampling of the bipartite eQTL network (shuffling the associations between SNPs and mRNAs). Deep permutations at 100,000 times were conducted on the Argonne Lab Beagle supercomputer to reach a sufficient power (20 million core hours). P-values were derived from the imputed results of the observed eQTL network and the set of permuted networks. False Discovery Rate (FDR) was used to adjust for multiplicity, and the SNP pairs with FDR<0.05 are termed prioritized Lead SNP pairs.

For MF and BP similarity calculations, a similar permutation procedure was conducted as done for mRNA overlap, except that SNPs and mRNAs without corresponding GO annotations were removed and only those with BP or MF associations remained in the bipartite network for resampling. The significance of overlapped GO terms from the SNP-GO-SNP triplets were further investigated for every pair of SNPs based on the same set of permutations and prioritized the overlapped terms between pairs of SNPs with a FDR<0.05. The whole procedure of permutations was conducted multiple times for different eQTL association cutoffs ranging from P≤10⁻⁴ to P≤10⁻⁶ and at three levels of node degrees: ND≥1, ND≤3, and ND≤5.

Through such stringent scale-free network controls, not only will the SNP pairs associated with same mRNAs be prioritized, but also the pairs in which two SNPs are associated with distinct mRNAs, if biological similarity exists.

Internal Validation: Enrichment Studies of Co-Classified Intergenic SNP Pairs Among Prioritized Pairs.

To demonstrate whether the shared biological mechanisms of intergenic Lead SNP pairs are relevant to the underlying biology of disease classes, it was assessed whether they are more likely to be found related to the same disease class than those across distinct classes. One-tailed Fisher's Exact Test (FET) was applied for the enrichment study, and odds ratios of significant mRNA overlapping, MF, and BP similarities for SNP pairs associated with the same disease classes were calculated by FET at multiple eQTL p-value cutoffs and three levels of node degrees.

External Validation: ENCODE Regulatory Elements and Chromatin Interaction Enrichment of Co-Classified Prioritized Intergenic SNP Pairs.

The potential mechanisms at play for the prioritized SNP pairs were also investigated. It was evaluated whether regulatory mechanisms were more likely to occur in prioritized intergenic SNP pairs associated with the same disease class as compared to their counterparts (distinct classes or insignificant). Encyclopedia of DNA Elements (ENCODE) data of Lead SNPs was integrated and Fisher's Exact Test was conducted to assess the enrichment of molecular regulations within prioritized SNP pairs of the same disease classes (Consortium et al., Nature, 2012, 489(7414): 57-74). Three possible shared regulatory mechanisms were assessed for pairs of SNPs located in distinct regions, including (1) binding with same transcription factor (via ChIP-seq), (2) binding with distinct transcription factors (via ChIP-seq) connecting through protein-protein interaction (PPI), and (3) within the anchor regions of long range chromatin interactions (via ChIA-PET (Djebali et al., Nature, 2012, 489(7414): 101)). The enrichment of regulatory mechanisms were compared with two conventional methods, which prioritized SNP pairs by (1) any intergenic Lead SNP pairs and (2) intergenic Lead SNP pairs with at least one mRNA overlap (non-statistical mRNA overap) in eQTL associations, respectively. To avoid loss of information when calculating regulatory functions between Loci in ENCODE, every Lead SNP was extended to its strongly associated LD SNPs based on the RegulomeDB database (inheritable haplotype) (Boyle et al., Genome research, 2012, 22(9): 1790-1797).

Overall Results and Visualization.

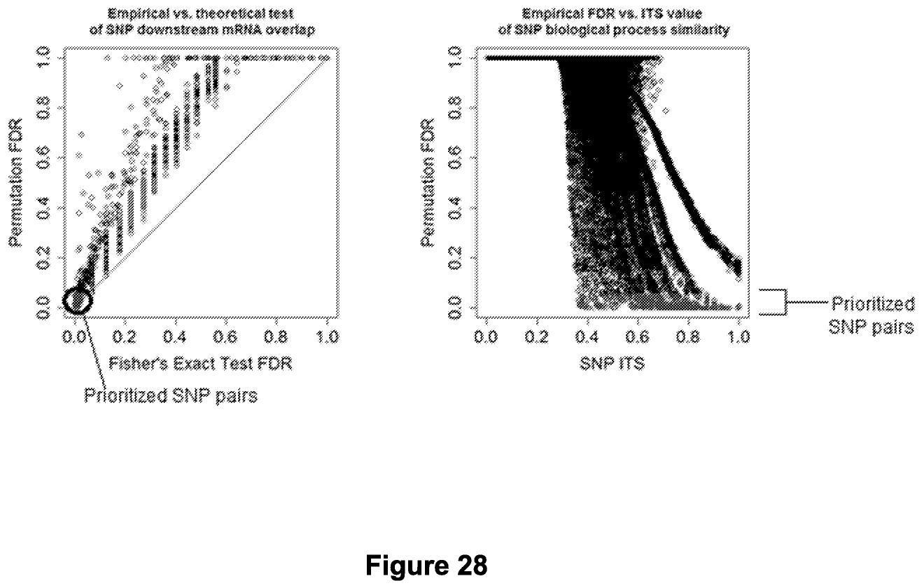

Prioritization of convergent downstream mechanisms of SNPs required extensive conservative scale-free permutation resampling of network edges (node degrees constant), shown substantially more conservative than conventional theoretical statistics or similarity-scores cutoffs (FIG. 28). 3,870 intergenic Lead SNP pairs (1,378 intergenic-intergenic; 2,492 intergenic-intragenic) were prioritized at FDR<0.05 that share at least one of the three imputed biological mechanisms, of which 755 pairs are found within the same disease class (280 intergenic-intergenic pairs; 475 intergenic-intragenic; 80 were associated with the same diseases). Without additional prioritization, the network relates these 755 pairs with as many as 1,683 mRNAs and 2,060 GO terms. After convergent mechanism prioritization, these SNP pairs implicate 14 disease classes, 277 Lead SNPs (167 intergenic, 98 noncoding intragenic, 12 protein-coding), 230 mRNAs, and 134 GO mechanisms.

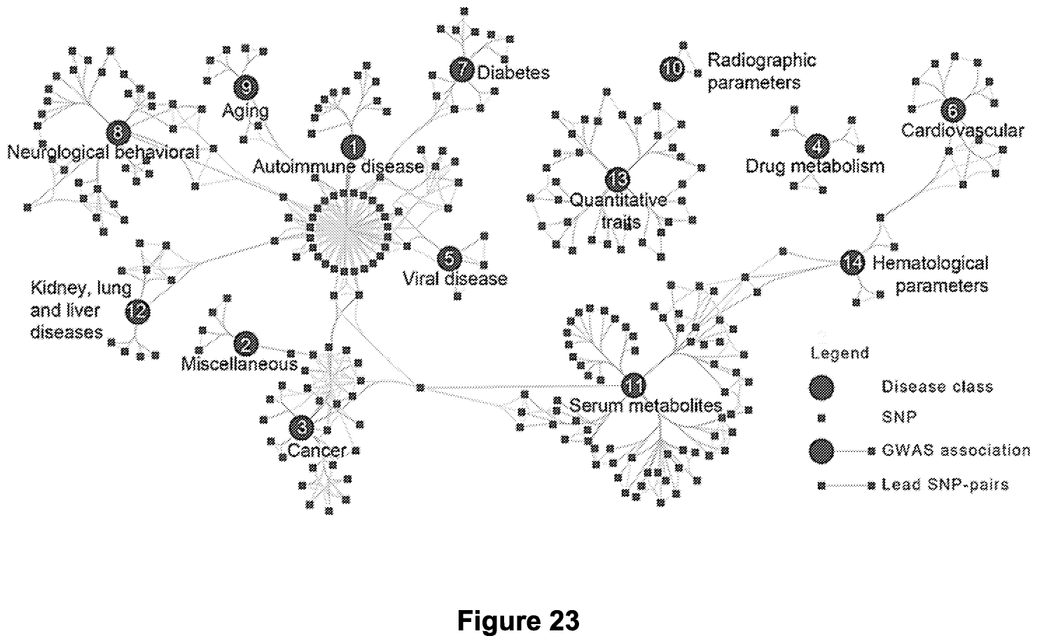

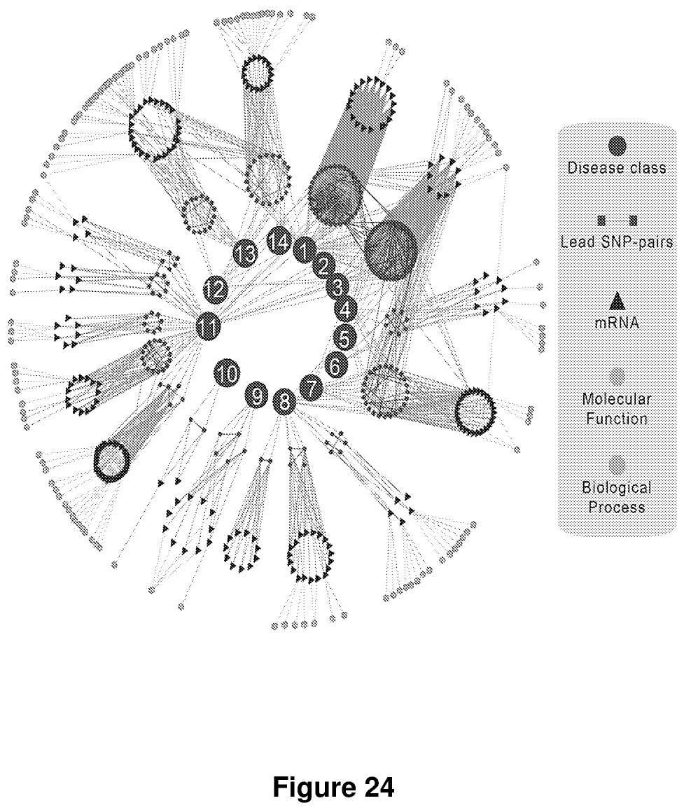

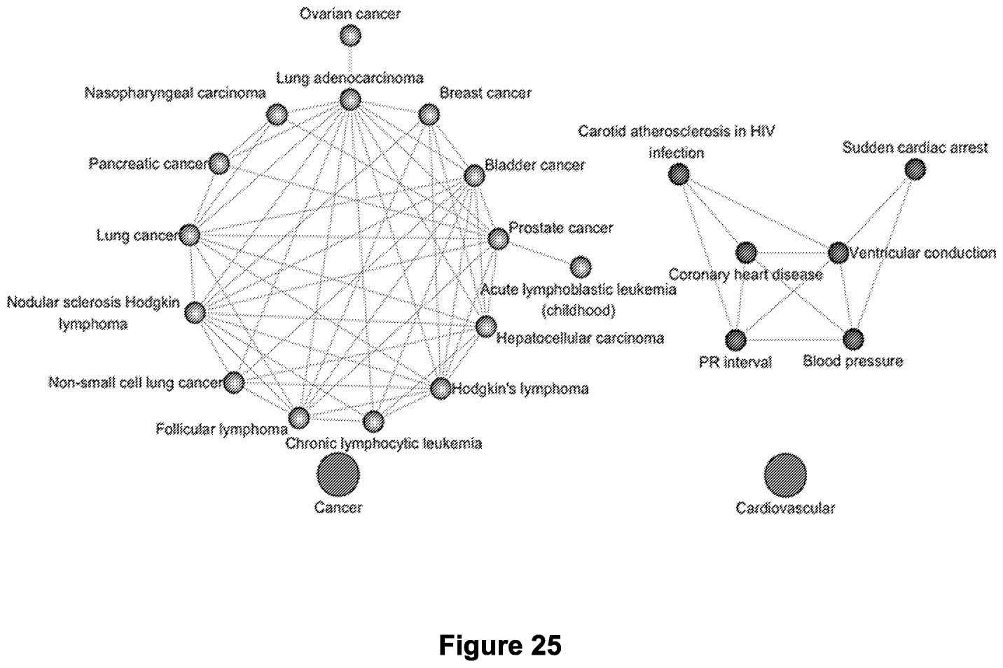

A simplified network shows only the 755 prioritized intergenic Lead SNP pairs and their related disease classes, leaving out the mRNAs and GO-terms for simplicity (FIG. 23). 14 of the 15 studied disease classes harbor convergent biological processes and molecular functions perturbed by a set of intergenic SNPs with similar downstream effects, presenting an apparent modularity for each class. A sub-network of prioritized biological mechanisms are further shown for the prioritized SNP pairs associated with the same classes in FIG. 24. The convergent connections among intergenic SNPs of distinct diseases within the same disease class suggest the investigation of an unusual form of pleiotropy: distinct intergenic risks of co-classified disease sharing common downstream mechanisms that could affect the same target transcripts that may relate to the emergence of both diseases in the same pathophysiological classification (e.g., FIG. 25 showing the detail of co-classified diseases associated through SNP pair similarity in FIG. 23, only cancer and cardiovascular system shown).

Enrichment of Shared Biological Mechanisms in Prioritized Intergenic SNP Pairs of Distinct Co-Classified Diseases.

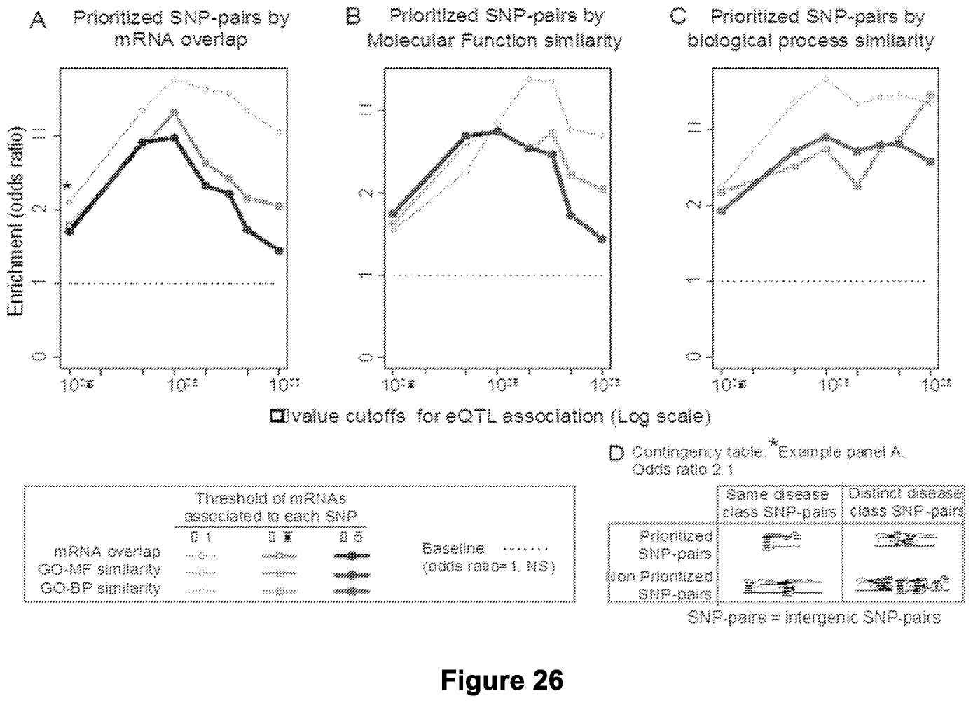

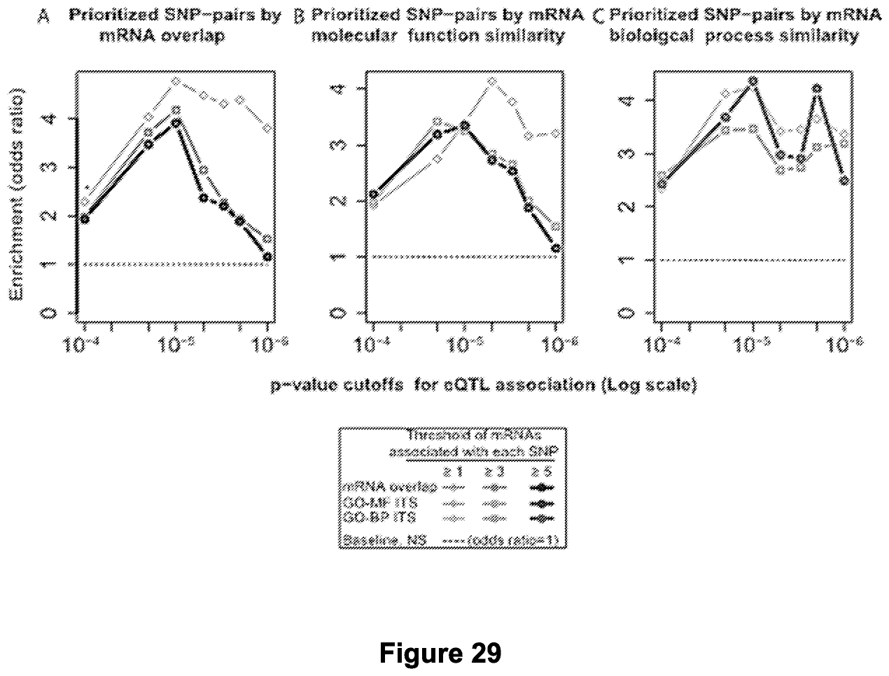

It was investigated whether intergenic Lead SNP pairs, with each SNP associated with two distinct co-classified diseases, were more likely to share a biological mechanism (prioritized) than SNP pairs associated with distinct diseases classified in distinct pathophysiological classes. Enrichment analyses were performed for the 755 prioritized SNP pairs associated with same classes among 3,870 prioritized intergenic Lead SNP pairs at different eQTL p-value cutoffs (10⁻⁶≤eQTL p-value≤10⁻⁴; 100,000 permutation resampling, SNP pair FDR<0.05) and different node degrees SNP node degree (count of mRNAs associated with that SNP at the eQTL p-value cutoff). As shown in FIG. 26, odds ratios (ORs) range from 1.4 to 3.8 (x-axis: 5.1×10⁻⁶≤p-value≤0.02), 1.4 to 3.4 (6.5×10⁻²⁶≤P≤2.1×10⁻²), and 1.9 to 3.7 (8.3×10⁻⁴≤P≤2.2×10⁻⁷) for mRNA overlapping, MF similarity, and BP similarity, respectively. This internal validation supports the hypothesis that biological mechanisms are more likely to be shared within a class of diseases and may define in part a common pathophysiology of otherwise distinct diseases. FIG. 29 similarly shows enrichment of shared mechanisms among the subset of intergenic-intergenic Lead SNP pairs associated with the same disease classes.

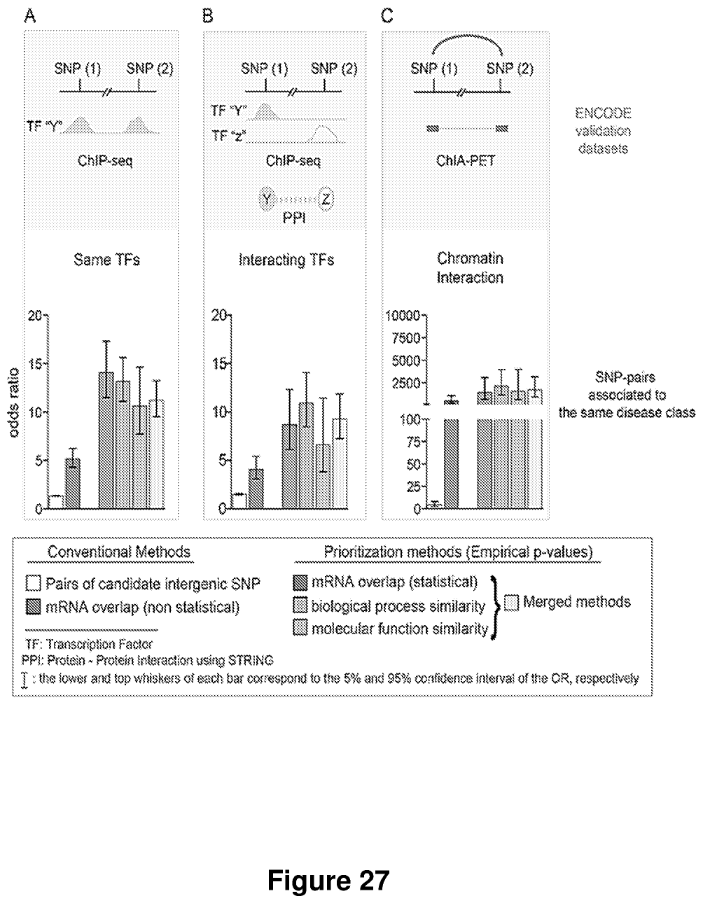

Enrichment of ENCODE regulatory elements and chromatin interaction in prioritized intergenic SNP pairs of distinct co-classified diseases. ENCODE data provides an opportunity to question if convergent candidate mechanisms of prioritized SNP pairs of co-classified diseases imputed by eQTL associations may be attributed to common regulatory elements (e.g., transcriptional factors) or long-range chromatin interactions. If so, this could be suggestive of possible epistasis between disease risks of distinct co-classified diseases, in other words, a disease class epistasis. Substantial enrichment in three types of regulatory elements were identified: shared transcription factor (FIG. 27, panel A), interacting transcription factors (FIG. 27, panel B), and long-range chromatin interactions in the region of the SNPs in the pair (FIG. 27, panel C). However, the effect size (odds ratio) of enrichment of regulatory elements in SNP pairs associated with distinct co-classified diseases shown in the figure is about 30 percent smaller than that of previously published enrichment of SNP pairs associated with the same disease (not shown in the figure) (Achour et al., NPJ Genomic Medicine, 2016, 1: 16006). Taken together, these results indicate that common regulatory mechanisms of intergenic SNPs strongly underpin the pathogenesis of a disease and to a moderate degree some mechanisms are also shared by distinct, yet pathophysiologically co-classified diseases.

### CONCLUSION

Using the quantified measurement of SNP biological similarity, 755 intergenic SNP pairs were identified that were associated by convergent eQTL function to distinct, yet pathophysiologically co-classified diseases. It was found that these independently inherited (LD r²<0.01) intergenic SNP pairs were more likely to be enriched in (i) shared transcription factors, (ii) interacting transcription factors, and (iii) long-range chromatin interactions. GWAS was able to identify about half the variants in intergenic regions. However the array platforms are seeded biasedly with half the probes in intergenic regions (selection bias). This proposes that more than 80% of the complex-disease associated variants could be located in intergenic regions, suggesting that if the heritability gap is attributable to genetic interactions, the majority of these would occur with intergenic noncoding regions. This prioritized network implies complex epistasis between intergenic polymorphisms of co-classified diseases and offers a roadmap for a novel therapeutic paradigm: repositioning medications that target proteins within downstream mechanisms of intergenic disease-associated SNPs.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations, which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

We claim:

1. A method for identifying a drug able to therapeutically treat a disease, the method comprising:
a) retrieving a list of single-nucleotide polymorphisms (SNPs) associated with one or more diseases from a first database, and generating a set of disease risk SNPs;
b) retrieving a list of gene products from a second database, wherein said gene products have altered expression associated with one or more of the disease risk SNPs;
c) selecting two or more disease risk SNPs which share at least one gene product having altered expression from the second database, and generating a set of one or more SNP pairs;
d) analyzing the one or more SNP pairs to determine similarity of biological mechanisms between each SNP within a SNP pair, and generating prioritized SNP pairs where higher prioritized pairs have increased similarity of biological mechanisms; and
e) identifying a drug able to interact with the shared gene product having altered expression from a prioritized SNP pair.

2. The method of claim 1 wherein the step of identifying a drug comprises retrieving a list describing a plurality of drugs and molecular targets affected by each of the plurality of drugs from a drug database.

3. The method of claim 1 wherein the drug is selected from a plurality of drug candidates approved to treat a different disease.

4. The method of claim 1 further comprising searching one or more additional databases for biological processes, molecular function, regulatory factors, or combinations thereof, associated with each of the disease SNPs, and determining the similarity of biological mechanisms based, in part, on similarity of the biological processes, molecular function, regulatory factors, or combinations thereof, from the one or more additional databases.

5. The method of claim 4 wherein the one or more additional databases comprise a gene ontology molecular function database, a gene ontology biological process database, any functional and regulatory database, or combinations thereof.

6. The method of claim 1 wherein the first database is any genetic database and the second database is a gene/RNA expression database.

7. The method of claim 1 wherein the altered gene product is a protein or mRNA molecule.

8. The method of claim 1 wherein analyzing the SNP pairs comprises determining whether each SNP of a SNP pair is within a protein-coding region of a gene or is outside a protein-coding region of a gene; and prioritizing SNP pairs having at least one SNP outside a protein-coding region of a gene.

9. The method of claim 1 further comprising testing the drug for therapeutic effect against the disease.

10. A method for simulating the effects of a therapeutic drug treatment on a patient or a group of patients, the method comprising:
a) screening a tissue sample extracted from a patient selected to receive a drug for single-nucleotide polymorphisms (SNPs),
b) identifying gene products associated with the SNPs from the patient tissue sample having altered expression;
c) retrieving from one or more databases information on altered gene product expression associated with adverse or beneficial effects of the drug;
d) simulating the adverse or beneficial drug effect occurring when the drug is administered to the patient based on the gene products that are associated with both the SNPs from the patient tissue sample and the adverse or beneficial effects of the drug.

11. The method of claim 10 wherein the gene products are proteins or mRNA molecules.

12. The method of claim 10 wherein the one or more databases comprise an expression database.

13. The method of claim 10 further comprising changing the drug to be administered to the patient when an adverse drug effect is predicted to occur.

14. A non-transitory computer-readable storage medium having a software program containing computer-executable instructions for performing a method for identifying drug candidates to treat a selected disease, said method comprising:
a) searching a first database for single-nucleotide polymorphisms (SNPs) associated with presence of the selected disease, and generating a set of disease SNPs;
b) searching a second database for gene products having altered expression associated with each of the disease SNPs;
c) selecting two or more disease SNPs which share at least one gene product having altered expression from the second database, and generating a set of one or more SNP pairs;
d) analyzing the one or more SNP pairs and generating a similarity value for each SNP pair based on similarity of biological mechanisms between each SNP within the SNP pair;
e) generating a set of prioritized SNP pairs where higher prioritized pairs have a higher similarity value; and
f) identifying a drug able to interact with the shared gene product having altered expression from a prioritized SNP pair.

15. The computer-readable storage medium of claim 14 wherein the computer-readable storage medium is a computer processor or server accessible by other computers through a computer network.

16. The computer-readable storage medium of claim 14 wherein analyzing the one or more SNP pairs comprises searching one or more additional databases for biological processes, molecular function, regulatory factors, or combinations thereof, associated with each of the disease SNPs, and determining the similarity of biological mechanisms based, in part, on similarity of the biological processes, molecular function, regulatory factors, or combinations thereof, from the one or more additional databases.

17. A pharmacogenomic system for identifying drug candidates to treat a disease comprising:
a) a disease SNP module comprising information which identifies a plurality of SNPs associated with presence of the disease;
b) an altered gene product module comprising information on gene products having altered expression and the plurality of SNPs;
c) a pairing module connected to the disease SNP module and the altered gene product module able to generate a set of one or more SNP pairs where both SNPs in each pair are associated with a same gene product having altered expression;
d) an analyzing module connected to the pairing module able to predict similarity of biological mechanisms between each SNP within a generated SNP pair, and able to generate a set of prioritized SNP pairs having increased similarity of biological mechanisms;
e) a drug and molecular target module comprising information on a plurality of drugs and molecular targets affected by each of the plurality of drugs; and
f) an identification module able to identify one or more drugs from the plurality of drugs which affects a molecular target which is the same as the gene product having altered expression from a prioritized SNP pair.

18. The pharmacogenomic system of claim 17 wherein the disease SNP module, altered gene product module, and drug and molecular target module are, independently from one another, part of a computer system, computer network, or software program executable by a computer processor.

19. The pharmacogenomic system of claim 17 further comprising display means for displaying the generated SNP pairs, prioritized SNP pairs, drugs and molecular targets, and identified drug candidates.

20. The pharmacogenomic system of claim 17 wherein the disease SNP module, altered gene product module, and drug and molecular target module are able to connect to one or more databases over a computer network.
